SEQUENCE LISTING (81 sequences)

SEQ ID NO: 1 - DNA, 2622 bp, Saccharomyces cerevisiae
  CDS (599)..(2179)
actcatacgctgaggaagaggaattttaaggttcactctcgcccccaacctccttatcctttctatatttcaagtatattgtaaaaaaaaaaattccccctactgttatatctctttggtgatatcttatctataatctacttattatatttctctaatatctatgtacaaaaaataaaaaaaaaaaccattctccttctctaacctgcatttgatgaaagcttagtaagtctcatcgtagttgatgggccaaagacatttgtacttgtttcatcagattcttacatcacccatttccatgattatttcacggatttcattagtaattgcatttttgtatccctagttccttttctcccgccttgccgtcgaattgaaaaaaaaaatggaaatgaggacatcttccattgatatccaggacaaagcaagagtagaagtaactattagcaaggagaattccaaaaatagtgaacttcaaggtaagcagatctatagtttcaatatctgattttctgcttttcaaaacttaaaattcattagaaggtaaaacaagaacccgtaaaattaaagttaccgatattatcaggtttttcaccccatgtctactatcccatttagaaagaactatgtgttcaaaaactgggccggaatttattctgcaaaaccagaacgttacttccaaccaagttcaattgatgaggttgtcgagttagtaaagagtgccaggctagctgaaaaaagcttagttactgttggttcgggccattctcctagtaacatgtgcgttactgatgaatggcttgttaacttagacagattggacaaagtacaaaagtttgttgaatatcctgagttacattatgccgatgtcacagttgatgccggtatgaggctttaccaattgaatgaatttttgggtgcgaaaggttactctatccaaaatttaggctctatctcagaacaaagtgttgctggcataatctctactggtagtcatggttcctcaccttatcacggtttgatttcttctcaatacgtaaacttgactattgttaatggtaagggcgaattgaagttcttggatgctgaaaacgatccagaagtctttaaagctgctttactttcagttggaaaaattggtatcattgtctctgctactatcagggttgttcccggcttcaatattaaatccactcaagaagtgattacttttgaaaaccttttgaagcaatgggataccctatggacttcatctgaatttatcagagtttggtggtacccttatactagaaaatgtgttctatggaggggtaacaaaactacagatgcccaaaatggtccagccaagtcatggtggggtaccaagctgggtagatttttctacgaaactctattatggatctctaccaaaatctatgcgccattaaccccatttgtggaaaagttcgttttcaacaggcaatacgggaaattggagaagagctctactggtgatgttaatgttaccgattctatcagcggatttaatatggactgtttgttttcacaatttgttgatgaatgggggtgccctatggataatggtttggaagtcttacgttcattggatcattctattgcgcaggctgccataaacaaagaattttatgtccacgtgcctatggaagtccgttgctcaaatactacattaccttctgaacccttggatactagcaagagaacaaacaccagtcccggtcccgtttatggcaatgtgtgccgcccattcctggataacacaccatcccattgcagatttgctccgttggaaaatgttaccaacagtcagttgacgttgtacataaatgctaccatttataggccgtttggctgtaatactccaattcataaatggtttaccctttttgaaaatactatgatggtagcgggaggtaagccacattgggccaagaacttcctaggctcaaccactctagctgctggaccagtgaaaaaggatactgattacgatgactttgaaatgagggggatggcattgaaggttgaagaatggtatggcgaggatttgaaaaagttccggaaaataagaaaggagcaagatcccgataatgtattcttggcaaacaaacagtgggctatcataaatggtattatagatcctagtgagttgtccgactagtctctttttgtctcaataatctctatattttactaaaaaagaatatatatatatatatatatagcagtgtgatgactgttcatgtacattctaataactattcctagctgcctatcaaagactttttttttgaattagagctttttagtaatcatgggacccttttttcttttcattatccttactatagttttttttttttggaaaagccgaacgcggtaatgattggtcgtataagcaaaaacgaaacatcggcatggcgtaacgtagatccatctacagggaagtttttagaaatcagatagaaatgtattttgagtgctgtatatattgcagtactttttttctctctaggatttaagtatgtttagtattaactcatatcacattttttctttgtaaaaagcaaccattcgcaacaatgtcgatagtagagacatgca

SEQ ID NO: 2 - protein, 526 aa, Saccharomyces cerevisiae
MSTIPFRKNYVFKNWAGIYSAKPERYFQPSSIDEVVELVKSARLAEKSLVTVGSGHSPSNMCVTDEWLVNLDRLDKVQKFVEYPELHYADVTVDAGMRLYQLNEFLGAKGYSIQNLGSISEQSVAGIISTGSHGSSPYHGLISSQYVNLTIVNGKGELKFLDAENDPEVFKAALLSVGKIGIIVSATIRVVPGFNIKSTQEVITFENLLKQWDTLWTSSEFIRVWWYPYTRKCVLWRGNKTTDAQNGPAKSWWGTKLGRFFYETLLWISTKIYAPLTPFVEKFVFNRQYGKLEKSSTGDVNVTDSISGFNMDCLFSQFVDEWGCPMDNGLEVLRSLDHSIAQAAINKEFYVHVPMEVRCSNTTLPSEPLDTSKRTNTSPGPVYGNVCRPFLDNTPSHCRFAPLENVTNSQLTLYINATIYRPFGCNTPIHKWFTLFENTMMVAGGKPHWAKNFLGSTTLAAGPVKKDTDYDDFEMRGMALKVEEWYGEDLKKFRKIRKEQDPDNVFLANKQWAIINGIIDPSELSD

SEQ ID NO: 3 - DNA, 1323 bp, Mus musculus
  CDS (1)..(1323)
atggtccatgggtacaaaggggtccagttccaaaactgggcgaagacctatggctgcagtccagagatgtactaccagcccacatcagtgggggaggtcagagaggtgctggccctggcccggcagcagaacaagaaagtgaaggtggtgggtggcggccactcgccttcagacatcgcctgcaccgatggcttcatgattcacatgggcaagatgaaccgggttctccaggtggacaaggagaagaagcaggtcacagtggaagccggtatcctcctgactgacctgcacccacagctggacaagcatggcctggccctgtctaatctgggagccgtgtctgatgtgacggttggtggcgtcattgggtctggaacacataacaccgggatcaagcacggtatcctggccacccaggtggtggccctgaccctgatgaaggctgatggaacagttctggaatgttctgagtcaagtaatgcagatgtgttccaggctgcaagggtgcacctgggctgcctgggtgttatcctcactgtcaccctgcagtgtgtgccacagttccaccttctggagacatcctttccttcgaccctcaaggaggtccttgacaacctggacagccacctgaagaagtctgagtacttccgcttcctctggtttcctcacagtgagaacgtcagcatcatctaccaagatcacaccaacaaggagccctcctctgcatctaactggttttgggactatgccattgggttctacctcctggaattcttgctctggaccagcacctacctgccacgcctcgtgggctggatcaaccgcttcttcttctggctgctgttcaactgcaagaaggagagcagcaacctcagccacaagatcttctcctacgagtgtcgcttcaagcagcatgtccaagactgggccatccccagggagaagaccaaggaggccctgctggagctaaaggccatgctggaggcccaccccaaggtggtagcccactaccccgtggaggtgcgcttcacccgaggtgatgacatcctgctgagcccgtgcttccagagggacagctgctacatgaacatcattatgtacaggccctatgggaaggatgtgcctcggttggattactggctggcctatgagaccatcatgaagaagtttggaggcaggccccactgggcaaaggcccacaattgcaccaggaaggactttgagaaaatgtaccccgcctttcacaagttctgtgacatccgcgagaagctggaccccactggaatgttcttgaattcgtacctggaaaaggttttctactaa

SEQ ID NO: 4 - protein, 440 aa, Mus musculus
MVHGYKGVQFQNWAKTYGCSPEMYYQPTSVGEVREVLALARQQNKKVKVVGGGHSPSDIACTDGFMIHMGKMNRVLQVDKEKKQVTVEAGILLTDLHPQLDKHGLALSNLGAVSDVTVGGVIGSGTHNTGIKHGILATQVVALTLMKADGTVLECSESSNADVFQAARVHLGCLGVILTVTLQCVPQFHLLETSFPSTLKEVLDNLDSHLKKSEYFRFLWFPHSENVSIIYQDHTNKEPSSASNWFWDYAIGFYLLEFLLWTSTYLPRLVGWINRFFFWLLFNCKKESSNLSHKIFSYECRFKQHVQDWAIPREKTKEALLELKAMLEAHPKVVAHYPVEVRFTRGDDILLSPCFQRDSCYMNIIMYRPYGKDVPRLDYWLAYETIMKKFGGRPHWAKAHNCTRKDFEKMYPAFHKFCDIREKLDPTGMFLNSYLEKVFY

SEQ ID NO: 5 - DNA, 2027 bp, Arabidopsis thaliana
  CDS (57)..(1595)
atcctgtacaagaacatttccctcaggttcagatcgcctgaagttaaacaaaaaaaatgctccggtcacttcttctccgacgctccgtcggccattctctcggaaccctatctccgtcttcatccaccatccgttcctcattttcgcctcatcgtactctctgcaccaccggtcaaacattgacaccaccaccgccgccaccgccacgtcctccacctccgcctccggccaccgcctcagaagctcaattccgtaaatacgccggatacgcagcactcgctatcttctctggagttgctacctatttctcatttccattccctgagaatgctaaacacaagaaggctcaaatcttccgttacgctcctttacctgaagatcttcacactgtctctaattggagtggtactcatgaggtacagactaggaactttaatcaaccggagaatcttgctgatctcgaagctcttgttaaggaatctcatgagaagaagttaaggattcgtcccgttggatcgggtctctcgcctaatgggattggtttgtctcgctctgggatggtgaatctggcgcttatggataaagttctagaggtggataaagagaagaagagagttacggtgcaggctgggattagggtccagcaattggttgacgccattaaagactatggtcttactcttcagaactttgcctccattagagagcagcagattggtggtattattcaggttggggcacatgggacaggtgctagattgcctcctattgatgagcaggtgatcagtatgaagctggttactcctgcgaagggaacaattgaactttcaagagagaaagatccggagctctttcatctagctcgatgtggccttggtggacttggagttgttgctgaggtcaccctccaatgcgttgcaagacatgaacttgtggaacacacatacgtctcaaacttgcaagaaatcaagaaaaatcacaaaaaattgctctctgcaaacaagcatgttaagtacctatatattccttataccgacacagtcgtggttgtaacatgcaatcctgtatcaaaatggagtgggccacctaaggacaaaccaaagtacactacagatgaggctgtacagcatgtccgtgatctctacagagagagcattgtgaagtatagggtccaggactctggtaagaagtctcctgacagcagtgagccagacatacaggagctttcatttacagagttgagagacaaactacttgcccttgatcctctcaatgacgttcacgttgcaaaagtaaatcaagctgaggcagagttttggaaaaaatcagaaggatatagagtagggtggagtgatgaaattctgggctttgactgtggtggtcagcagtgggtgtcagaatcttgttttcctgctggaaccctcgccaaccctagcatgaaagaccttgaatacatagaagagctgaaaaaactaatagaaaaggaagcaataccagcacctgctccaatagagcagcgatggacagctcgaagttggtataatcatgtacctcccgacagcagaccctcgccagagaaaggacatcacagatgaatttttccactatagacatttgacacagaaacaattgtgggatcaattttctgcgtatgaacattgggctaaaattgagataccaaaagacaaagaagaacttgaagccttacaggcaagaataagaaaacgtttcccagtggatgcatacaacaaagcacgtagggagctggacccaaatagaatcctctccaacaacatggtggaaaagctcttcccagtctccaccactgcttaaaacattttcatcaataggttttttcctctttttgggatcctatacatctttttattgcatctataagtttttttaagaagaggtgaggaatgagtgaatccaatatatttgtcatatttgtcacaaaactgtacattgagttttaccactcttttcacagttcctatgaataaatatttgttctttactttctc

SEQ ID NO: 6 - protein, 512 aa, Arabidopsis thaliana
MLRSLLLRRSVGHSLGTLSPSSSTIRSSFSPHRTLCTTGQTLTPPPPPPPRPPPPPPATASEAQFRKYAGYAALAIFSGVATYFSFPFPENAKHKKAQIFRYAPLPEDLHTVSNWSGTHEVQTRNFNQPENLADLEALVKESHEKKLRIRPVGSGLSPNGIGLSRSGMVNLALMDKVLEVDKEKKRVTVQAGIRVQQLVDAIKDYGLTLQNFASIREQQIGGIIQVGAHGTGARLPPIDEQVISMKLVTPAKGTIELSREKDPELFHLARCGLGGLGVVAEVTLQCVARHELVEHTYVSNLQEIKKNHKKLLSANKHVKYLYIPYTDTVVVVTCNPVSKWSGPPKDKPKYTTDEAVQHVRDLYRESIVKYRVQDSGKKSPDSSEPDIQELSFTELRDKLLALDPLNDVHVAKVNQAEAEFWKKSEGYRVGWSDEILGFDCGGQQWVSESCFPAGTLANPSMKDLEYIEELKKLIEKEAIPAPAPIEQRWTARSWYNHVPPDSRPSPEKGHHR

SEQ ID NO: 7 - DNA, 1121 bp, Pyrenophora tritici-repentis
  CDS (1)..(1074)
atggcttccaatacgcattcaacaaatggacatgaaatcgacagctcaaaacctactatcttacatttgggtgaagatatccggtggaatcacgaattatacgaggaattaaagagggagttcaatattgtgaggacatactcaatgaacagatcagagttcaagaaggccttggcggaaaagcagtggggagactttgttgggatgtaccggccgttctggaatacaggtggagagatgggtaactggaatgaagaacttatttctcttctcccctcttcctgcaaaatctacgcttcagctggcgcaggcttcgattgggttgatactcgtgccctcgctgcccatggaaccatatactgcaacagtgcctctgcatgcaccgaatctgtcgctgacacggccatcgtgcttattctatcttgctaccgcgacatcacctggtctttccttgcagcaagatcttgcgatccccaggaattccgcaatgccaaccagaatattgcggctgtgacacacaatccgaatggcagtacgcttggcattatcggactcgggcgaatcggtcttcgaatcgcgcaaaaggcgacaagggcattcgagatgaagattgcgtattatgacgttatcaggatggaggagagagagaaagaggtgggggctaggtggtgcgctacactgggtgaacttttgtctacttctgattgtgtgcttgtggctacaccgtttgaaggtgagacactgctttcgacggagcagtttgggaagttcaaaaagggcgcgagtcttgtcaatattgcgagggggaagctgattgacgaaaacgcgctggttgaggccatggacaagggcattgtggctgcagcgggcttggatgttcacgcggatgaaccaaatgtgaatcccaagttggccaagagaagcaatgtcatggtcttgagtcatacggcgggcgcgagtgtagaaagtcatgtagggtttgagaggttgggtatggagaacttgctgggttggaagagggagggtcgtagaggatgtgtcagcgcagtgaacttgcagtggctgaaggagtagccaaagtcttctcatagtggggtagaagaaatgcacaaggttaagct

SEQ ID NO: 8 - protein, 357 aa, Pyrenophora tritici-repentis
MASNTHSTNGHEIDSSKPTILHLGEDIRWNHELYEELKREFNIVRTYSMNRSEFKKALAEKQWGDFVGMYRPFWNTGGEMGNWNEELISLLPSSCKIYASAGAGFDWVDTRALAAHGTIYCNSASACTESVADTAIVLILSCYRDITWSFLAARSCDPQEFRNANQNIAAVTHNPNGSTLGIIGLGRIGLRIAQKATRAFEMKIAYYDVIRMEEREKEVGARWCATLGELLSTSDCVLVATPFEGETLLSTEQFGKFKKGASLVNIARGKLIDENALVEAMDKGIVAAAGLDVHADEPNVNPKLAKRSNVMVLSHTAGASVESHVGFERLGMENLLGWKREGRRGCVSAVNLQWLKE

SEQ ID NO: 9 - DNA, 2860 bp, Homo sapiens
  CDS (242)..(1846)
ggggcgctggtgtgatcgagctcacgtagcgagggctgcagtcgcctcctccctggcgctgccatcgcggcctagaggttataaaagggctaacgggctccctctgctgcccagtcgcgccgccagcgggctgagggtaggaagtagccgctccgagtggaggcgactgggggctgaagagcgcgccgccctctcgtcccactttccaggtgtgtgatcctgtaaaattaaatcttccaagatgatctggtatatattaattataggaattctgcttccccagtctttggctcatccaggcttttttacttcaattggtcagatgactgatttgatccatactgagaaagatctggtgacttctctgaaagattatattaaggcagaagaggacaagttagaacaaataaaaaaatgggcagagaagttagatcggctaactagtacagcgacaaaagatccagaaggatttgttgggcatccagtaaatgcattcaaattaatgaaacgtctgaatactgagtggagtgagttggagaatctggtccttaaggatatgtcagatggctttatctctaacctaaccattcagagacagtactttcctaatgatgaagatcaggttggggcagccaaagctctgttacgtctccaggatacctacaatttggatacagataccatctcaaagggtaatcttccaggagtgaaacacaaatcttttctaacggctgaggactgctttgagttgggcaaagtggcctatacagaagcagattattaccatacggaactgtggatggaacaagccctaaggcaactggatgaaggcgagatttctaccatagataaagtctctgttctagattatttgagctatgcggtatatcagcagggagacctggataaggcacttttgctcacaaagaagcttcttgaactagatcctgaacatcagagagctaatggtaacttaaaatattttgagtatataatggctaaagaaaaagatgtcaataagtctgcttcagatgaccaatctgatcagaaaactacaccaaagaaaaaaggggttgctgtggattacctgccagagagacagaagtacgaaatgctgtgccgtggggagggtatcaaaatgacccctcggagacagaaaaaactcttttgccgctaccatgatggaaaccgtaatcctaaatttattctggctccagctaaacaggaggatgaatgggacaagcctcgtattattcgcttccatgatattatttctgatgcagaaattgaaatcgtcaaagacctagcaaaaccaaggctgagccgagctacagtacatgaccctgagactggaaaattgaccacagcacagtacagagtatctaagagtgcctggctctctggctatgaaaatcctgtggtgtctcgaattaatatgagaatacaagatctaacaggactagatgtttccacagcagaggaattacaggtagcaaattatggagttggaggacagtatgaaccccattttgactttgcacggaaagatgagccagatgctttcaaagagctggggacaggaaatagaattgctacatggctgttttatatgagtgatgtgtctgcaggaggagccactgtttttcctgaagttggagctagtgtttggcccaaaaaaggaactgctgttttctggtataatctgtttgccagtggagaaggagattatagtacacggcatgcagcctgtccagtgctagttggcaacaaatgggtatccaataaatggctccatgaacgtggacaagaatttcgaagaccttgtacgttgtcagaattggaatgacaaacaggcttccctttttctcctattgttgtactcttatgtgtctgatatacacatttcctagtcttaactttcaggagtttacaattgactaacactccatgattgattcagtcatgaacctcatcccatgtttcatctgtggacaattgcttactttgtgggttcttttaaaagtaacacgaaatcatcatattgcataaaaccttaaagttctgttggtatcacagaagacaaggcagagtttaaagtgaggaattttatatttaaagaactttttggttggataaaaacataatttgagcatccagttttagtatttcactacatctcagttggtgggtgttaagctagaatgggctgtgtgataggaaacaaatgccttacagatgtgcctaggtgttctgtttacctagtgtcttactctgttttctggatctgaagactagtaataaactaggacactaactgggttccatgtgattgccctttcatatgatcttctaagttgatttttttcctcccaagtcttttttaaagaaagtatactgtattttaccaaccccctctcttttcttttagctcctctgtggtgaattaaacgtacttgagttaaaatatttcgattttttttttttttttaatggaaagtcctgcataacaacactgggccttcttaactaaaatgctcaccacttagcctgtttttttatcccttttttaaaatgacagatgattttgttcaggaattttgctgtttttcttagtgctaataccttgcctcttattcctgctacagcagggtggtaatattggcattctgattaaatactgtgccttaggagactggaagtttaaaaatgtacaagtcctttcagtgatgagggaattgattttttttaaaagtctttttcttagaaagccaaaatgtttgtttttttaagattctgaaatgtgttgtgacaacaatgacctatttatgatcttaaatcttttttaaaaaaaaaaaaaaaa

SEQ ID NO: 10 - protein, 534 aa, Homo sapiens
MIWYILIIGILLPQSLAHPGFFTSIGQMTDLIHTEKDLVTSLKDYIKAEEDKLEQIKKWAEKLDRLTSTATKDPEGFVGHPVNAFKLMKRLNTEWSELENLVLKDMSDGFISNLTIQRQYFPNDEDQVGAAKALLRLQDTYNLDTDTISKGNLPGVKHKSFLTAEDCFELGKVAYTEADYYHTELWMEQALRQLDEGEISTIDKVSVLDYLSYAVYQQGDLDKALLLTKKLLELDPEHQRANGNLKYFEYIMAKEKDVNKSASDDQSDQKTTPKKKGVAVDYLPERQKYEMLCRGEGIKMTPRRQKKLFCRYHDGNRNPKFILAPAKQEDEWDKPRIIRFHDIISDAEIEIVKDLAKPRLSRATVHDPETGKLTTAQYRVSKSAWLSGYENPVVSRINMRIQDLTGLDVSTAEELQVANYGVGGQYEPHFDFARKDEPDAFKELGTGNRIATWLFYMSDVSAGGATVFPEVGASVWPKKGTAVFWYNLFASGEGDYSTRHAACPVLVGNKWVSNKWLHERGQEFRRPCTLSELE

SEQ ID NO: 11 - DNA, 2596 bp, Homo sapiens
  CDS (198)..(1724)
gagcctcgaagtccgccggccaatcgaaggcgggccccagcggcgcgtgcgcgccgcggccagcgcgcgcgggcgggggggcaggcgcgccccggacccaggatttataaaggcgaggccgggaccggcgcgcgctctcgtcgcccccgctgtcccggcggcgccaaccgaagcgccccgcctgatccgtgtccgacatgctgcgccgcgctctgctgtgcctggccgtggccgccctggtgcgcgccgacgcccccgaggaggaggaccacgtcctggtgctgcggaaaagcaacttcgcggaggcgctggcggcccacaagtacctgctggtggagttctatgccccttggtgtggccactgcaaggctctggcccctgagtatgccaaagccgctgggaagctgaaggcagaaggttccgagatcaggttggccaaggtggacgccacggaggagtctgacctggcccagcagtacggcgtgcgcggctatcccaccatcaagttcttcaggaatggagacacggcttcccccaaggaatatacagctggcagagaggctgatgacatcgtgaactggctgaagaagcgcacgggcccggctgccaccaccctgcctgacggcgcagctgcagagtccttggtggagtccagcgaggtggctgtcatcggcttcttcaaggacgtggagtcggactctgccaagcagtttttgcaggcagcagaggccatcgatgacataccatttgggatcacttccaacagtgacgtgttctccaaataccagctcgacaaagatggggttgtcctctttaagaagtttgatgaaggccggaacaactttgaaggggaggtcaccaaggagaacctgctggactttatcaaacacaaccagctgccccttgtcatcgagttcaccgagcagacagccccgaagatttttggaggtgaaatcaagactcacatcctgctgttcttgcccaagagtgtgtctgactatgacggcaaactgagcaacttcaaaacagcagccgagagcttcaagggcaagatcctgttcatcttcatcgacagcgaccacaccgacaaccagcgcatcctcgagttctttggcctgaagaaggaagagtgcccggccgtgcgcctcatcaccctggaggaggagatgaccaagtacaagcccgaatcggaggagctgacggcagagaggatcacagagttctgccaccgcttcctggagggcaaaatcaagccccacctgatgagccaggagctgccggaggactgggacaagcagcctgtcaaggtgcttgttgggaagaactttgaagacgtggcttttgatgagaaaaaaaacgtctttgtggagttctatgccccatggtgtggtcactgcaaacagttggctcccatttgggataaactgggagagacgtacaaggaccatgagaacatcgtcatcgccaagatggactcgactgccaacgaggtggaggccgtcaaagtgcacagcttccccacactcaagttctttcctgccagtgccgacaggacggtcattgattacaacggggaacgcacgctggatggttttaagaaattcctggagagcggtggccaggatggggcaggggatgatgacgatctcgaggacctggaagaagcagaggagccagacatggaggaagacgatgatcagaaagctgtgaaagatgaactgtaatacgcaaagccagacccgggcgctgccgagacccctcgggggctgcacacccagcagcagcgcacgcctccgaagcctgcggcctcgcttgaaggagggcgtcgccggaaacccagggaacctctctgaagtgacacctcacccctacacaccgtccgttcacccccgtctcttccttctgcttttcggtttttggaaagggatccatctccaggcagcccaccctggtggggcttgtttcctgaaaccatgatgtactttttcatacatgagtctgtccagagtgcttgctaccgtgttcggagtctcgctgcctccctcccgcgggaggtttctcctctttttgaaaattccgtctgtgggatttttagacatttttcgacatcagggtatttgttccaccttggccaggcctcctcggagaagcttgtcccccgtgtgggagggacggagccggactggacatggtcactcagtaccgcctgcagtgtcgccatgactgatcatggctcttgcatttttgggtaaatggagacttccggatcctgtcagggtgtcccccatgcctggaagaggagctggtggctgccagccctggggcccggcacaggcctgggccttccccttccctcaagccagggctcctcctcctgtcgtgggctcattgtgaccactggcctctctacagcacggcctgtggcctgttcaaggcagaaccacgacccttgactcccgggtggggaggtggccaaggatgctggagctgaatcagacgctgacagttcttcaggcatttctatttcacaatcgaattgaacacattggccaaataaagttgaaattttaccacctgtaaaaaaaaaaaaaaaaaa

SEQ ID NO: 12 - protein, 508 aa, Homo sapiens
MLRRALLCLAVAALVRADAPEEEDHVLVLRKSNFAEALAAHKYLLVEFYAPWCGHCKALAPEYAKAAGKLKAEGSEIRLAKVDATEESDLAQQYGVRGYPTIKFFRNGDTASPKEYTAGREADDIVNWLKKRTGPAATTLPDGAAAESLVESSEVAVIGFFKDVESDSAKQFLQAAEAIDDIPFGITSNSDVFSKYQLDKDGVVLFKKFDEGRNNFEGEVTKENLLDFIKHNQLPLVIEFTEQTAPKIFGGEIKTHILLFLPKSVSDYDGKLSNFKTAAESFKGKILFIFIDSDHTDNQRILEFFGLKKEECPAVRLITLEEEMTKYKPESEELTAERITEFCHRFLEGKIKPHLMSQELPEDWDKQPVKVLVGKNFEDVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDMEEDDDQKAVKDEL

SEQ ID NO: 13 - DNA, 3715 bp, Homo sapiens
  CDS (389)..(2515)
atccacttgtacaaccagaagggagctcacgtgtaaagcgcctcgtccccggccggcctcaagcggcgctagctctctggctggcgctggagtgtgccgcccctggcagccccgccgctgcagcggggagcctgggagagcggctctcggagtctcaggcagcaggaggagcgtgtgagctgtgggcgtccctttaagagcggctggccaggcacggcctccgcctctcagtacgcggagcgccggcggtcacctggggctcgcggagcggccagatcgcggcggagtcggcgcgcttccccgagggaaggtgggagaggggacccggacgcgaggtgccccgaagccctctcgagcgtaaccgtcccgcgcctctctgaggcggaggatgcgggagcgcatctgggcgccgccgctgctgctgctgctgccgctgctactgccgccgccactgtggggcggccccccggacagcccacgccgggagctggagctggagcccgggcctctgcagcccttcgacctgctctacgccagcggcgcggccgcctactacagcggagactacgagcgagcggtgcgcgacttggaagcggcgctgcgcagccaccggcgcctgcgggaaatccgcacgcgctgtgcccgccactgcgcggcgcgccacccgctcccgcccccgccccccggcgagggccccggcgctgagctgccccttttccgctccttgttggggcgggcgcgctgttatcgcagctgtgagacccagcgcctcgggggccccgcatcccgccaccgcgtcagcgaggatgtgcgcagcgacttccagcgcagagtgccctacaactacctgcagcgggcctacatcaagcttaaccagctcgaaaaagcagtggaagcagctcacacatttttcgtggctaaccctgagcacatggaaatgcagcagaacattgagaattacagggcgacagctggtgttgaagcattgcagttggtagacagagaagccaagccacacatggagagttacaatgcaggagttaaacattatgaggctgatgactttgagatggctatcaggcacttcgaacaagccttaagagaatatttcgttgaagatacagaatgccggaccctatgtgaggggcctcagagatttgaagaatatgagtatttagggtataaggctggtctgtatgaagctattgcagatcactacatgcaggtgcttgtttgtcagcatgaatgtgtgagggaacttgccacccgccctggccgcctctctcccatcgagaattttcttcctctgcactatgattacctacagtttgcctactatcgagttggtgagtatgtgaaagccctggagtgtgccaaagcctatcttctatgccatccagatgatgaggatgtcctagacaatgtggattactatgagagtctgctggatgatagcattgacccggcatccattgaggccagagaggatttaacaatgtttgtgaaacgtcataagctggagtctgagctgataaaatcagctgcagaaggtctggggttttcatacactgaaccgaattattggatcagatatggaggacgacaggatgagaatcgggtcccttcaggagtgaacgtagagggagcagaagttcatggattctcaatgggaaaaaagctatcacccaagatagatcgagacctaagagaaggtggtcctctactctatgagaacatcacattcgtctacaactcggagcagctgaacgggactcagcgggttctcctggataacgtcctgtcggaagaacagtgccgggagctccacagcgtggccagtggaatcatgcttgttggtgatggatacagaggaaaaacttcaccccatacacccaatgaaaagtttgaaggtgcaactgtcctgaaagcactcaaatctggttatgaaggtcgagtcccactgaagagcgctcgtctgttttatgacatcagcgaaaaggctcgaaggattgtagaatcttattttatgctgaactcaactctgtatttttcctatacacacatggtctgccgaacagccctgtctggtcagcaggatagaagaaatgacctcagtcatcccatccatgctgacaactgtttgttggatccagaggccaacgaatgctggaaggagcctcctgcttacacatttcgagactatagtgctctcctatatatgaatgatgactttgaaggaggagaattcatattcacagagatggatgctaagactgtgactgcctctataaaaccaaaatgtgggcgcatgatcagcttctcatctggaggagagaaccctcatggggtgaaggcagtcaccaagggaaagaggtgtgctgtggctctgtggttcaccttggacccactttatagagaattggagcgaatacaggctgatgaagtgattgcaattctggatcaagaacagcaagggaagcatgaactgaatatcaaccctaaagatgagctataaaaatgagaaagaatgttctatcaaatatttatttaaattgttaatcttatgagaacctttttatttttgtacagagccatggtataaattaacaggttaatgtcagtcatcagatcttccttctcttcctaaggatgcttgtgttgcctcaatctatcaatctatctttcttgttttgggttgttttctctctctctctctctctctctcttcttagagacatggtctaaccatgttgtctaggatatagggcagtggctattcacagatgtgatgatagcacactggagcctcaaactcttaggctcaggcgatccttcaagcctcccggggagctgggaccacaggcacgtgccaccacacccagctctctttcttggtttttcatcatttcatgtatctatcaaagcccagttcacctcctcccccaaacacacacacacacacacacacacacacacacacacacaattaagttgctgcaaattcaaaagcttagagagaataagcttcttggtggtgaaactacaactctcacgtgtgctccagttctaaaattaacctgtgcctggtctctgaagccctttcttgctctgtgcctttcagccacatccttaggtgctaacggccatgagctccgactctccaaagtgagctccactttgggtctgaggagcccctggcagagtccacgctgcctcaggtatcatgggcgtaatgatcacccaggctccgggagatctcatggatgattactgtatgagacagaggggacttcagtctttccagggccttggtggaatttttggctctggtgttttcgccagacaataaacttacactggaagctttgattcaccctccacagtactccagaaaggactgtcctataagttgtacactttaaaaggtcatgtagaggttgtagtagaatggcttttcaccctggtgactttggaagaaactcttgaatactgcctgcatccgggcaccatggccaggttgcctaggagtggggtccactgatgaaaagaggtgttttgtacttacataagaaaaataaatttctgattgattttaaccgtcatctgcttatattttgggggcccctcctcattgctgctatccagcacacagatttgtgcttgtgtctgatttgtttaataaagggaggcttattttaaaaaaaaaaaaaaaaaa

SEQ ID NO: 14 - protein, 708 aa, Homo sapiens
MRERIWAPPLLLLLPLLLPPPLWGGPPDSPRRELELEPGPLQPFDLLYASGAAAYYSGDYERAVRDLEAALRSHRRLREIRTRCARHCAARHPLPPPPPGEGPGAELPLFRSLLGRARCYRSCETQRLGGPASRHRVSEDVRSDFQRRVPYNYLQRAYIKLNQLEKAVEAAHTFFVANPEHMEMQQNIENYRATAGVEALQLVDREAKPHMESYNAGVKHYEADDFEMAIRHFEQALREYFVEDTECRTLCEGPQRFEEYEYLGYKAGLYEAIADHYMQVLVCQHECVRELATRPGRLSPIENFLPLHYDYLQFAYYRVGEYVKALECAKAYLLCHPDDEDVLDNVDYYESLLDDSIDPASIEAREDLTMFVKRHKLESELIKSAAEGLGFSYTEPNYWIRYGGRQDENRVPSGVNVEGAEVHGFSMGKKLSPKIDRDLREGGPLLYENITFVYNSEQLNGTQRVLLDNVLSEEQCRELHSVASGIMLVGDGYRGKTSPHTPNEKFEGATVLKALKSGYEGRVPLKSARLFYDISEKARRIVESYFMLNSTLYFSYTHMVCRTALSGQQDRRNDLSHPIHADNCLLDPEANECWKEPPAYTFRDYSALLYMNDDFEGGEFIFTEMDAKTVTASIKPKCGRMISFSSGGENPHGVKAVTKGKRCAVALWFTLDPLYRELERIQADEVIAILDQEQQGKHELNINPKDEL

SEQ ID NO: 15 - DNA, 7102 bp, Homo sapiens
  CDS (3157)..(4437)
ttaggggcagaaaaacatttgtaataattaatggctttgagagacacaaggctttgtttgccccagagtattagttaacccacctagtgctcctaatcatacaatattaaggattgggagggacattcattgcctcactctctatttgtttcaccttctgtaaaattggtagaataatagtacccacttcatagcattgtatgatgattaaattggttaatatttttaaaatgcttagaacacagattgggcacataacagcaagcaccacatgtgtttataagataaattcctttgtgttgccttccgttaaagtttaaataagtaaataaataaataaatacttgcatgacattttgaagtctctctataacatctgagtaagtggcggctgcgacaatgctactggagttccagaatcgtgttggtgacaagattgttcaccagcatatggtgtggtgaaaactcactaatttggaattagttcagattattaagcctgaataggtgaaaatcctgaaatcaaggatctttggaactatttgaaatcagtattttatattttcctgttgtattcattaaagtgttgcaagtgttctatttgatggattaagtatatttaggatatacatgttcaatttgtgattttgtatacttaattggaacaagaaagctaataaaggttttgatatggacatctattcttttaagtaaacttcaatgaaaatatatgagtagagcatatagagatgtaaataatttgtggacacaccacagactgaaatagcaaatttaaaagaaattgttggaagaatcaagtgtttgtggaatgagtcctcctagtaaagttcctgctcttgtgaataattaagcctcatgtataattactatagcaaaaggaagcctaagaagtattagactctacttgtatttaaattacattttacataatttatgtgtatgaaaaatgttttaaatgcttattttcgtaagccatgagatagctcctttatattttaagaatttctgaattaatttgcttggattttattagtgcaaatggcagagctagcaattcctttttctgtgttcccattccatcctattcatccctcttttaggaaactctgaactctggattgtccttgtttacatacctgcctcctgcattggactatgtgtctctgagtgtagtatgactaattcatttgtttgtcaaggactctcaatgcatttgttgaacagcctaattagtaatgtctgcaacaatgacattttactgtatttaataaagctctgggaaagtaggatacacataagacaggtctaggtctaaattctttacagaaacttggatttttagttcggtttgaaatttgaagatgtgagtatatttatctcagtttcccaaaggacaagctaattggaattatcatcctctttcacttgattggatccccagaatgccatttacgcatgcagcaggattttataacagttttaaattctgtatatttgatgaagaggttttatatttttggattcaagcctctttttaaacttctacaatatggtttacaataattccttatatcctgcttttgaaatacatattacaactttttaagtttggaaggctatatttcaaggactgaagttacagtatactcaagtgatacacaagcctagcaccccactttccacatagtgttcgataaagattgataaactcgaaatcacagaccttttaattcttaagacaaatagcagcagaaagaaacatctttggcttatttctggtaaggtttttatgctctgtaaaacaaagaattgtattcatccgcgcagcacagattctattaaaaataaatgtgagagtcgttaatgtagtactgctcatttaccatcaaaattcacttttcaggaataatcccatcagtttaaattggatattggaatgagcattgattacatttaacttggtagcccaaaatttcttcatggggttttgaactcggcgggatttcaaaggttttaaaaatgagtttttgattttttttaaaaccctcaaatttcattacctttaaactaggtcgaaacggggcgcaagagattggattaacaccatagtaatacttattttgttcttaaccatttcagggcttcttgaaatagaggctgtatggtgtaatggaaaaaacagccttggaatctgggagcctgattcctggattcagtcccagttttgcgtgaccttgggcaagttactttacttctctgaatttccgtttcctcctctgcaaaatgaggatcgcaatagccaccttgcaaccttgactggagcgagcctcgcacaccccgcgccggcctggaggaagagcagccatgattacgccgccttcgctccgctacccgcttgcggctggcgccctcctccagcaggtgtaggcgctgccgcgctgccccacgcctttccgccgctcgcgggcctgcgcctcggcgtccccgaggaggccgctgcgggctgaggtagcgcaccggcctctcggcgtcccagtccggtcccgggcggagggaaagcgggcgacccacctccgaggcagaagccgaggcccggccccgccgagtgcggaggagcgcaggcagcccccgcccctcggccctccccccggccctcccggccctccctccgccccctccgccctcgcgcgccgcccgcccgggtcgccgcggggccgtggtgtacgtgcagagcgcgcagagcgagtggcgcccgtatgccctgcgctcctccacagcctgggccgggccgcccgggacgctgaggcggcggcggcggccgagggggccggtcttgcgctccccaggcccgcgcgcctgagcccaggttgccattcgccgcacaggccctattctctcagccctcggcggcgatgaggcgctgaggcggctgccggcgctgcgccggagcttaggactcggaagcggccgggccgagggcgtggggtgccggcctccctgaggcgagggtagcgggtgcatggcgcagtaacggcccctatctctctccccgctccccagcctcgggcgaggccgtccggccgctacccctcctgctcggccgccgcagtcgccgtcgccgccgccgccgccgccatggccaatgacagcggcgggcccggcgggccgagcccgagcgagcgagaccggcagtactgcgagctgtgcgggaagatggagaacctgctgcgctgcagccgctgccgcagctccttctactgctgcaaggagcaccagcgtcaggactggaagaagcacaagctcgtgtgccagggcagcgagggcgccctcggccacggagtgggcccacaccagcattccggccccgcgccgccggctgcagtgccgccgcccagggccggggcccgggagcccaggaaggcagcggcgcgccgggacaacgcctccggggacgcggccaagggaaaagtaaaggccaagcccccggccgacccagcggcggccgcgtcgccgtgtcgtgcggccgccggcggccagggctcggcggtggctgccgaagccgagcccggcaaggaggagccgccggcccgctcatcgctgttccaggagaaggcgaacctgtaccccccaagcaacacgcccggggatgcgctgagccccggcggcggcctgcggcccaacgggcagacgaagcccctgccggcgctgaagctggcgctcgagtacatcgtgccgtgcatgaacaagcacggcatctgtgtggtggacgacttcctcggcaaggagaccggacagcagatcggcgacgaggtgcgcgccctgcacgacaccgggaagttcacggacgggcagctggtcagccagaagagtgactcgtccaaggacatccgaggcgataagatcacctggatcgagggcaaggagcccggctgcgaaaccattgggctgctcatgagcagcatggacgacctgatacgccactgtaacgggaagctgggcagctacaaaatcaatggccggacgaaagccatggttgcttgttatccgggcaatggaacgggttatgtacgtcatgttgataatccaaatggagatggaagatgtgtgacatgtatatattatcttaataaagactgggatgccaaggtaagtggaggtatacttcgaatttttccagaaggcaaagcccagtttgctgacattgaacccaaatttgatagactgctgtttttctggtctgaccgtcgcaaccctcatgaagtacaaccagcatatgctacaaggtacgcaataactgtttggtattttgatgcagatgagagagcacgagctaaagtaaaatatctaacaggtgaaaaaggtgtgagggttgaactcaataaaccttcagattcggtcggtaaagacgtcttctagagcctttgatccagcaataccccacttcacctacaatattgttaactatttgttaacttgtgaatacgaataaatgggataaagaaaaatagacaaccagttcgcattttaataaggaaacagaaacaactttttgtgttgcatcaaacagaagattttgactgctgtgactttgtactgcatgatcaacttcaaatctgtgattgcttacaggaggaagataagctactaattgaaaatggtttttacatctggatatgaaataagtgccctgtgtagaatttttttcattcttatattttgccagatctgttatctagctgagttcatttcatctctcccttttttatatcaagtttgaatttgggataatttttctatattaggtacaatttatctaaactgaattgagaaaaaattacagtattattcctcaaaataacatcaatctatttttgtaaacctgttcatactattaaattttgccctaaaagacctcttaataatgattgttgccagtgactgatgattaattttattttacttaaaataagaaaaggagcactttaattacaactgaaaaatcagattgttttgtagtccttccttacactaatttgaactgttaaagattgctgctttttttttgacattgtcaataacgaaacctaattgtaaaacagtcaccatttactaccaataacttttagttaatgttttacaaggaaaaagacacaagaagagtttaaatttttttgttttgttttgtttttttgagacagtcttgctctgttacccaggctggaggggagtggtgcattcttggctcactgcaacctccgcctcccaggttcaagcaatcctcccacctcagcctcccaactagctgggactgcaggcacacaccaccatgcctgactaatttttgtatgtttagtagagacggggttttgccatgttgcctaggctggggtttaagttaaattttttaaaaaactaaagtgactggcactaagtgaacttgagattatcctcagcttcaagttcctaagataagggctttcttaagctttcaggtgtatgtatcctctagatgtagacaataatgtcccatttctaagtcttttccttttgcttctccttaaattgattgtacttccaaatttgctgttatgtttttttcctaatactgtgatctatctgatctgcagacaagaaccttgtctctgttgaagagcatcaaggggagattatgtacacattgaaactgaagtgtggtgttactgacggaatgtgcagtaactcctcagatatctgttaaggcatttcccagatgtgatgccagccttcttacctgtactgaaagatgcttagcttagaaaaaaacaaaacagatgcaaaatcagataattttattttgtttcatgggttttcttatttactttttaaacaaggaaggaatattagaaaatcacacaaggcctcacatacatgttatttaaagaatgaattgggacggatgtcttagacttcactttcctaggctttttagcaaaacctaaagggtggtatccatattttgcgtgaattatgggtgtaagaccttgcccacttaggttttctatctctgtccttgatcttctttgccaaaatgtgagtatacagaaattttctgtatatttcaacttaagacatttttagcatctgtatagtttgtattcaatttgagaccttttctatgggaagctcagtaatttttattaaaagattgccattgctattcatgtaaaacatggaaaaaaattgtgtagtgaagccaacagtggacttaggatgggattgaatgttcagtatagtgatctcacttaggagaatttgcaggagaaagtgatagtttattgttttttcctcgcccatattcagttttgttctacttcctccccttccttccagatgataacatcacatctctacagtaagtgcctctgccagcccaacccaggagcgcaagttgtctttgccatctggtctatagtacagtgcgcggcgttaggccacaactcaaaagcattatcttttttagggttagtagaaattgttttatgttgatgggaggtttgtttgattgtcaaaatgtacagccacagccttttaatttgggagcccctgttgtcattcaaatgtgtacctctacagttgtaaaaagtattagattctactatctgtgggttgtgcttgccagacaggtcttaaattgtatattttttggaaaagtttatatactctcttaggaatcattgtgaaaagatcaagaaatcaggatggccatttatttaatatccattcatttcatgttagtgggactattaacttgtcaccaagcaggactctatttcaaacaaaatttaaaactgtttgtggcctatatgtgtttaatcctggttaaagataaagcttcataatgctgtttttattcaacacattaaccagctgtaaaacacagacctttatcaagagtaggcaaagattttcaggattcatatacagatagactataaagtcatgtaatttgaaaagcagtgtttcattatgaaagagctctcaagttgcttgtaaagctaatctaattaaaaagatgtataaatgttgttgaaacattaaaaaa

SEQ ID NO: 16 - protein, 426 aa, Homo sapiens
MANDSGGPGGPSPSERDRQYCELCGKMENLLRCSRCRSSFYCCKEHQRQDWKKHKLVCQGSEGALGHGVGPHQHSGPAPPAAVPPPRAGAREPRKAAARRDNASGDAAKGKVKAKPPADPAAAASPCRAAAGGQGSAVAAEAEPGKEEPPARSSLFQEKANLYPPSNTPGDALSPGGGLRPNGQTKPLPALKLALEYIVPCMNKHGICVVDDFLGKETGQQIGDEVRALHDTGKFTDGQLVSQKSDSSKDIRGDKITWIEGKEPGCETIGLLMSSMDDLIRHCNGKLGSYKINGRTKAMVACYPGNGTGYVRHVDNPNGDGRCVTCIYYLNKDWDAKVSGGILRIFPEGKAQFADIEPKFDRLLFFWSDRRNPHEVQPAYATRYAITVWYFDADERARAKVKYLTGEKGVRVELNKPSDSVGKDVF

SEQ ID NO: 17 - DNA, 2995 bp, Homo sapiens
  CDS (457)..(2673)
taggcttcctccctccctgtcgccgccagcctctcgctctccccgtcttccgcccgcactctctgggcagggctgcggccaggacccgcccccgcgtcccgcccgaccctccgccaggggtcacttcccctgtccaggtttcagcttccacatgtgtcaagcggctggctcagcccagagtccctgtctcccgcccgccggcccgagccgccgcccctcccccgcctcccgtgcgcccgggacaatcctcgccttgtctgtggcgccggcatctggagctttctgtagcctccggatacgcctttttttcagggcgtagccccagccaagctgctccccgcggcggccgcacagcagcccgagcgccccctttccagagctcccctccggagctgggatccaggcgcgtagcggagatcccaggatcctgggtgctgtctgggcccgctccccaccatgacctcctcggggcctggaccccggttcctgctgctgctgccgctgctgctgccccctgcggcctcagcctccgaccggccccggggccgagacccggtcaacccagagaagctgctggtgatcactgtggccacagctgaaaccgaggggtacctgcgtttcctgcgctctgcggagttcttcaactacactgtgcggaccctgggcctgggagaggagtggcgagggggtgatgtggctcgaacagttggtggaggacagaaggtccggtggttaaagaaggaaatggagaaatacgctgaccgggaggatatgatcatcatgtttgtggatagctacgacgtgattctggccggcagccccacagagctgctgaagaagttcgtccagagtggcagccgcctgctcttctctgcagagagcttctgctggcccgagtgggggctggcggagcagtaccctgaggtgggcacggggaagcgcttcctcaattctggtggattcatcggttttgccaccaccatccaccaaatcgtgcgccagtggaagtacaaggatgatgacgacgaccagctgttctacacacggctctacctggacccaggactgagggagaaactcagccttaatctggatcataagtctcggatctttcagaacctcaacggggctttagatgaagtggttttaaagtttgatcggaaccgtgtgcgtatccggaacgtggcctacgacacgctccccattgtggtccatggaaacggtcccactaagctgcagctcaactacctgggaaactacgtccccaatggctggactcctgagggaggctgtggcttctgcaaccaggaccggaggacactcccgggggggcagcctcccccccgggtgtttctggccgtgtttgtggaacagcctactccgtttctgccccgcttcctgcagcggctgctactcctggactatccccccgacagggtcacccttttcctgcacaacaacgaggtcttccatgaaccccacatcgctgactcctggccgcagctccaggaccacttctcagctgtgaagctcgtggggccggaggaggctctgagcccaggcgaggccagggacatggccatggacctgtgtcggcaggaccccgagtgtgagttctacttcagcctggacgccgacgctgtcctcaccaacctgcagaccctgcgtatcctcattgaggagaacaggaaggtgatcgcccccatgctgtcccgccacggcaagctgtggtccaacttctggggcgccctgagccccgatgagtactacgcccgctccgaggactacgtggagctggtgcagcggaagcgagtgggtgtgtggaatgtaccatacatctcccaggcctatgtgatccggggtgataccctgcggatggagctgccccagagggatgtgttctcgggcagtgacacagacccggacatggccttctgtaagagctttcgagacaagggcatcttcctccatctgagcaatcagcatgaatttggccggctcctggccacttccagatacgacacggagcacctgcaccccgacctctggcagatcttcgacaaccccgtcgactggaaggagcagtacatccacgagaactacagccgggccctggaaggggaaggaatcgtggagcagccatgcccggacgtgtactggttcccactgctgtcagaacaaatgtgtgatgagctggtggcagagatggagcactacggccagtggtcaggcggccggcatgaggattcaaggctggctggaggctacgagaatgtgcccaccgtggacatccacatgaagcaggtggggtacgaggaccagtggctgcagctgctgcggacgtatgtgggccccatgaccgagagcctgtttcccggttaccacaccaaggcgcgggcggtgatgaactttgtggttcgctaccggccagacgagcagccgtctctgcggccacaccacgactcatccaccttcaccctcaacgttgccctcaaccacaagggcctggactatgagggaggtggctgccgcttcctgcgctacgactgtgtgatctcctccccgaggaagggctgggcactcctgcaccccggccgcctcacccactaccacgaggggctgccaacgacctggggcacacgctacatcatggtgtcctttgtcgacccctgacactcaaccactctgccaaacctgccctgccattgtgcctttttagggggcctggcccccgtcctgggagttgggggatgggtctctctgtctccccacttcctgagttcatgttccgcgtgcctgaactgaatatgtcaccttgctcccaagacacggccctctcaggaagctcccggagtccccgcctctctcctccgcccacaggggttcgtgggcacagggcttctggggactccccgcgtgataaattattaatgttccgcagtctcactctgaataaaggacagtttgtaagtcttgaaaaaaaaaaaaaaaaaaa

SEQ ID NO: 18 - protein, 738 aa, Homo sapiens
MTSSGPGPRFLLLLPLLLPPAASASDRPRGRDPVNPEKLLVITVATAETEGYLRFLRSAEFFNYTVRTLGLGEEWRGGDVARTVGGGQKVRWLKKEMEKYADREDMIIMFVDSYDVILAGSPTELLKKFVQSGSRLLFSAESFCWPEWGLAEQYPEVGTGKRFLNSGGFIGFATTIHQIVRQWKYKDDDDDQLFYTRLYLDPGLREKLSLNLDHKSRIFQNLNGALDEVVLKFDRNRVRIRNVAYDTLPIVVHGNGPTKLQLNYLGNYVPNGWTPEGGCGFCNQDRRTLPGGQPPPRVFLAVFVEQPTPFLPRFLQRLLLLDYPPDRVTLFLHNNEVFHEPHIADSWPQLQDHFSAVKLVGPEEALSPGEARDMAMDLCRQDPECEFYFSLDADAVLTNLQTLRILIEENRKVIAPMLSRHGKLWSNFWGALSPDEYYARSEDYVELVQRKRVGVWNVPYISQAYVIRGDTLRMELPQRDVFSGSDTDPDMAFCKSFRDKGIFLHLSNQHEFGRLLATSRYDTEHLHPDLWQIFDNPVDWKEQYIHENYSRALEGEGIVEQPCPDVYWFPLLSEQMCDELVAEMEHYGQWSGGRHEDSRLAGGYENVPTVDIHMKQVGYEDQWLQLLRTYVGPMTESLFPGYHTKARAVMNFVVRYRPDEQPSLRPHHDSSTFTLNVALNHKGLDYEGGGCRFLRYDCVISSPRKGWALLHPGRLTHYHEGLPTTWGTRYIMVSFVDP

SEQ ID NO: 19 - DNA, 5349 bp, Homo sapiens
  CDS (270)..(2546)
ggggcgtggccgtcgggcggcgagccgcgcgctctcgccccgggagggagtcacaggttcgtacacgcgaggccgggcgcgcgcagccggcaggcgggtgaagtcttcccaggtgctgcaggcggtgctgaggcacagggtctgctgcaggagcagcggcccgaacccgctccagcggcccgccgccgccagcggttcccgcgtcgcgtgtgtacccccgcgcactgaaggaggtccgccagccctcaccagcccccgcggaccgtgcaatggcccagcgtaagaatgccaagagcagcggcaacagcagcagcagcggctccggcagcggtagcacgagtgcgggcagcagcagccccggggcccggagagagacaaagcatggaggacacaagaatgggaggaaaggcggactctcaggaacttcattcttcacgtggtttatggtgattgcattgctgggcgtctggacatctgtagctgtcgtttggtttgatcttgttgactatgaggaagttctaggaaaactaggaatctatgatgctgatggtgatggagattttgatgtggatgatgccaaagttttattaggacttaaagagagatctacttcagagccagcagtcccgccagaagaggctgagccacacactgagcccgaggagcaggttcctgtggaggcagaaccccagaatatcgaagatgaagcaaaagaacaaattcagtcccttctccatgaaatggtacacgcagaacatgttgagggagaagacttgcaacaagaagatggacccacaggagaaccacaacaagaggatgatgagtttcttatggcgactgatgtagatgatagatttgagaccctggaacctgaagtatctcatgaagaaaccgagcatagttaccacgtggaagagacagtttcacaagactgtaatcaggatatggaagagatgatgtctgagcaggaaaatccagattccagtgaaccagtagtagaagatgaaagattgcaccatgatacagatgatgtaacataccaagtctatgaggaacaagcagtatatgaacctctagaaaatgaagggatagaaatcacagaagtaactgctccccctgaggataatcctgtagaagattcacaggtaattgtagaagaagtaagcatttttcctgtggaagaacagcaggaagtaccaccagaaacaaatagaaaaacagatgatccagaacaaaaagcaaaagttaagaaaaagaagcctaaacttttaaataaatttgataagactattaaagctgaacttgatgctgcagaaaaactccgtaaaaggggaaaaattgaggaagcagtgaatgcatttaaagaactagtacgcaaataccctcagagtccacgagcaagatatgggaaggcgcagtgtgaggatgatttggctgagaagaggagaagtaatgaggtgctacgtggagccatcgagacctaccaagaggtggccagcctacctgatgtccctgcagacctgctgaagctgagtttgaagcgtcgctcagacaggcaacaatttctaggtcatatgagaggttccctgcttaccctgcagagattagttcaactatttcccaatgatacttccttaaaaaatgaccttggcgtgggatacctcttgataggagataatgacaatgcaaagaaagtttatgaagaggtgctgagtgtgacacctaatgatggctttgctaaagtccattatggcttcatcctgaaggcacagaacaaaattgctgagagcatcccatatttaaaggaaggaatagaatccggagatcctggcactgatgatgggagattttatttccacctgggggatgccatgcagagggttgggaacaaagaggcatataagtggtatgagcttgggcacaagagaggacactttgcatctgtctggcaacgctcactctacaatgtgaatggactgaaagcacagccttggtggaccccaaaagaaacgggctacacagagttagtaaagtctttagaaagaaactggaagttaatccgagatgaaggccttgcagtgatggataaagccaaaggtctcttcctgcctgaggatgaaaacctgagggaaaaaggggactggagccagttcacgctgtggcagcaaggaagaagaaatgaaaatgcctgcaaaggagctcctaaaacctgtaccttactagaaaagttccccgagacaacaggatgcagaagaggacagatcaaatattccatcatgcaccccgggactcacgtgtggccgcacacagggcccacaaactgcaggctccgaatgcacctgggcttggtgattcccaaggaaggctgcaagattcgatgtgccaacgagaccaagacctgggaggaaggcaaggtgctcatctttgatgactcctttgagcacgaggtatggcaggatgcctcatctttccggctgatattcatcgtggatgtgtggcatccggaactgacaccacagcagagacgcagccttccagcaatttagcatgaattcatgcaagcttgggaaactctggagagaggctgcctttctggttccatctccttgggtgtgaggatagaatttcgaacaccaagagtcaattcccttgacttgcagcccgagtaattcaaagcctcctcctagggtcagaagacactaaagggaatatttgcctcgctgcaattcatttaggaaacaccctgctgtgtgtcatctcatgacagcactggtcttctgccagtatttaaggtgaacatttgatagcttctaccttaccagccaaagatattttttccacatagaataggtctaattcaatgtataatgagaacatatgtagaaactgtgaatggattgctttagtttgtaatttttctatgcagttatatttttctagtgtagctagactattttgtcatcatgtaccactacatttttgtttattttaatgacaagctgtataaatgctttacttctagctatttaatggtagcattactggggaactcagacttccctcttttaattcttcttagtaaaagatactcatgaaaaaagcagttttattttcctaacaaaaaagaaagagctcattatgtcagtgtctatgaactgtacccatcccaactctcaaatcgtttggttttttttatcttgattgagatcctcttctcactatgctagtggtggagatattgacaaaatcctatttctttcaaagaggaacttttcacaccgaaaaaagagcatggaattattttatattgttataaaaatcccagatgcaaatttttttaatgccaattattagagcttctggggaaaaagtatagttcacggaaataaaactatgttctttcagggttgggtggataggtggctgctagggtgtctggctcctggcggctttgccatccatgaggcaagggctgggaacacagtgtctttgcctatggtagatccatgtgaatgtcaggaagccagctcttcagtcttggagatgatttctgctacaattctgtagaaagattaaggatggcagagtaaaaggttaccaagaatgccaggatgtttttcttgggcgtaggaggtccagattactttcctttttgatgaaagagtttggaagactgtcccatctctctggcttgagaaatctctgccattttaaacatcactgtgaaatagcaattattatcatctgtatttagttttaacattacccacaacatagaaataataggtaaaaatcgtcttgcctactcattccaaagatgatcaagtcattaatctagcaaagtattcatgtatcagattttgtatattttgaatcaaagctaactaggaatgttagatataagaatgtaatgatattcatgcactgaattctaagccaatatgaacaaaaatgctgcatgaatggcacatataggtcaccaaagttcattcacaggtagaaaaaacttgtgctttcttttccatctaaaaacaaaaggagactttctttatctcatttaaagaacagctctttgaaattgaaattgaccctttttgcttgaccttaaggagattagcttccagtagatgagtttgcaaaatacttttcctgttcttttgttttgctggtattgaaaacatcccactaaatcagatgaagaggcatgggaggaaaaatatccaaattaattactaaaatcgagaagagaaggcaaactcttgaaaagtaaaaaggtgtttgtgaccttcagtatttattgaacagaggaaataactgacaagggcaatacaattcaatgttcatgtagtaacattcatgtcacttgttgaatttggttctcatatgtatattgcatacacataaattcaaactataagtcgtcatttttgagccatcatcttacattcatgtaatgaaattatggaagagagtaaaaactagctcttaacttagtaaatataatatggtatttaaaatcaggtcactacagtaaggttctaagtattgccaattgaaaagctagaaatggtattactgttgcaaagtgttgtcaataattgactccaatagcattgtaaatacttgtatcccacaactattttaaacccaagcaataaaatggattttctaattcacttcaattctttatttctcttacctatctatgtcttggtacataagaaaagtgtttccatcactgcattggtagaattatttcagtgttattatttttgtgatttcgtatgtctacacaaaaatgaattagtttaatttatattgtgatacagttgtttgagaaatatttttaattctgtttcaactttatttctccaagtgtataatataaaaattacttctgttattgttctctaccaataggggaaaaaattaaaacattagatccattgagaaagagatgatgtaataaataattaagattagtaataatattatcaggggtgattatgaccagttgaataatctctttcccttgaattatttagctaacaaattaactctcccaaatatttaaaataatgtaaaatcatattttactgcccattattaactaaaatatttttgtttgactttgagcaccaactggtaatactaataaatacccatgtcatgcagatggctgggcgaataagagatgtctaaaaatatgcactggtcttggaaaacatggcacaagtaaggatatcatatatgatgtctgtttattttatgtctgatttcttttgaatgagtagttggggactccatttctaaggagactaggtaaataaaatgacctttgacatttca

SEQ ID NO: 20 - protein, 758 aa, Homo sapiens
MAQRKNAKSSGNSSSSGSGSGSTSAGSSSPGARRETKHGGHKNGRKGGLSGTSFFTWFMVIALLGVWTSVAVVWFDLVDYEEVLGKLGIYDADGDGDFDVDDAKVLLGLKERSTSEPAVPPEEAEPHTEPEEQVPVEAEPQNIEDEAKEQIQSLLHEMVHAEHVEGEDLQQEDGPTGEPQQEDDEFLMATDVDDRFETLEPEVSHEETEHSYHVEETVSQDCNQDMEEMMSEQENPDSSEPVVEDERLHHDTDDVTYQVYEEQAVYEPLENEGIEITEVTAPPEDNPVEDSQVIVEEVSIFPVEEQQEVPPETNRKTDDPEQKAKVKKKKPKLLNKFDKTIKAELDAAEKLRKRGKIEEAVNAFKELVRKYPQSPRARYGKAQCEDDLAEKRRSNEVLRGAIETYQEVASLPDVPADLLKLSLKRRSDRQQFLGHMRGSLLTLQRLVQLFPNDTSLKNDLGVGYLLIGDNDNAKKVYEEVLSVTPNDGFAKVHYGFILKAQNKIAESIPYLKEGIESGDPGTDDGRFYFHLGDAMQRVGNKEAYKWYELGHKRGHFASVWQRSLYNVNGLKAQPWWTPKETGYTELVKSLERNWKLIRDEGLAVMDKAKGLFLPEDENLREKGDWSQFTLWQQGRRNENACKGAPKTCTLLEKFPETTGCRRGQIKYSIMHPGTHVWPHTGPTNCRLRMHLGLVIPKEGCKIRCANETKTWEEGKVLIFDDSFEHEVWQDASSFRLIFIVDVWHPELTPQQRRSLPAI

SEQ ID NO: 21 - DNA, 6928 bp, Homo sapiens
  CDS (76)..(1125)
ttttgtttttttttttttggggtccggaataggcggagcttccggttccggtgggggccgtccctggcggcggagatggcggcgacagcggcggaggctgtggcctctggctctggagagccccgggaggaggctggagccctcggccccgcctgggatgaatcccagttgcgcagttatagcttcccgactaggcccattccgcgtctgagtcagagcgacccccgggcagaggagcttattgagaatgaggagcctgtggtgctgaccgacacaaatcttgtgtatcctgccctgaaatgggaccttgaatacctgcaagagaatattggcaatggagacttctctgtgtacagtgccagcacccacaagttcttgtactatgatgagaagaagatggccaatttccagaactttaagccgaggtccaacagggaagaaatgaaatttcatgagttcgttgagaaactgcaggatatacagcagcgaggaggggaagagaggttgtatctgcagcaaacgctcaatgacactgtgggcaggaagattgtcatggacttcttaggttttaactggaactggattaataagcaacagggaaagcgtggctgggggcagcttacctctaacctgctgctcattggcatggaaggaaatgtgacacctgctcactatgatgagcagcagaacttttttgctcagataaaaggttacaaacgatgcatcttattccctccggatcagttcgagtgcctctacccataccctgttcatcacccatgtgacagacagagccaggtggactttgacaatcccgactacgagaggttccctaatttccaaaatgtggttggttacgaaacagtggttggccctggtgatgttctttacatcccaatgtactggtggcatcacatagagtcattactaaatggggggattaccatcactgtgaacttctggtataagggggctcccacccctaagagaattgaatatcctctcaaagctcatcagaaagtggccataatgagaaacattgagaagatgcttggagaggccttggggaacccacaagaggtggggcccttgttgaacacaatgatcaagggccgatacaactagcctgccaggggtcaaggcctcctgccaggtgactgctatcccgtccacaccgcttcattgatgaggacaggagactccaagcgctagtattgcacgctgcacttaatggactggactcttgccatggcccaggagtcaggtgtttggagcgaggcagggcagttggcactccactcctatttggagggacttcatacccttgcctcttgtgccccagcaccttctctctctgccccccgcctaaagtcctgcattcagtgtgtggagtcccagcttttggttgtcatcatgtctgtgtgtatgttagtctgtcaacttcggaatgtgtgcgtgtgtgtgcatgcacacgcatgtatgtatctgttccctgttccttctgggtcaggctgtcacttccggctctcagccctatctcctgcaacctcagtgcctcagcctgagagagagatgagatgctcttggactccccactgcatctgggctgcagggccagagctagtctgaccattaggtcagtctgcctcctgacagtttttgcgtagtcaagctctaggcggtatgggaatggctaccgggactctaatggggtgaaagagaggggaggcttgcctttgagagcctatatagccttcctgtgagagaggattagatagggttccaactgggcctacaagctcaagccatacataaaaggaccttgggacataagaaccaatgattgtgcataagttctaaattagagacacatatagtttctctctttcagcaccagctcttgcccctatgctgggtaccaagggagttctcctagctgtggcttctctaggttctaggggtgcaagcctctgtgtgtttgtttgtgtgtgtctgtgtgtgcgtatccacactaggggtgcaagcctctgggtgtgtgtgtgtgtgtgcgtgcgtgtgtgtgtgtgtgtccgtgtgtgtgtgtgtgtgtgtccacactggccagcctccctacttaccaaggttctccactgcttaccttttccagtgggacagtacagtgtgagcccccgggaagtactgcctgacctatcctaagcttttacacttggattttagccatcatatgttggccaggtctcactgcagcctgcccgaggctaactggctagagcctccaggccctatgatgctccctgcccaggccatatcctttattcctgctgagcttcctggctgaatagatgaaatggggtcaagcccaggcagctcattcactacctgtgatccacctcagggcacgggcaaacacataggcttgcgtcttaaagccagctcctctgccagaccccgttgtaatgtgccacaacaccctcaatagtcagggcaactggtggagcatggaagtcgaatttccttttctgttaggagctactcctgggaacccctctcagggctgcagcttacaggtgggcagctgtgattgcacaacttgaagggccatcattcacatctattcagtgggagtggggtccctgggattgggcagtgtggtggccctgtgtctcctcacctctgctcctgtcttcatcaccttctctctggaagggaagaggagttggaaggtctctggttttcttttctttttttttttttttgccaaaggtttacttccagcatctgagctctggctctcacccctgaagctcagttatagtgcactgatgaactgagaggatgcgtgtggatgtgtgtgcatgcctgagtgcgttttttggggaggggtgtttatttttagtaccccattctggggttctctgatgcagtgtggatgtgaagatatggtaccttctcaagtgtagctctttcaaatatagtcaatgctgggaaatgtgattgcagtgatctctatctctccacttcttttgggaaagaggagcacaggagcggaggaaaaacttggccacagtcacacttggaaagatagtagattattttcgttctcctcagcaggtctgctgtattcctcgctcagcgctcaaagatgtgggtgatgtgtcaaggaggcatgcactcagctggtccttgctgagttatttgctgacacaggtctcactaaggtggctgaggggtgggagggagaaggctaatcttggaaccttcacaggattggccttgatccttgggtaacacaagagtgattcctgatttttagacacctaatatgtgcctagtgcttttctctttgttcctcactatcctgcaaggtaggtattctcacttacagatgaataaatgggctcagagagattaggtgattagtgacaatcagttgtaacttagcactgttaggctgcaaaccccttgctccttcctttccaccaaactatgttgattttccttccttacctccctccttgtaccacaggttctgtcctcaagatatcaccccactgggcagtatctgaaggcagacccagcccaagatggtgcagaatatacagctcagggtgaaagttcttctgggctcctgctaacaaagtcttggaaaaactgcctgggaagccatattatgaattagtgctagaagaagctgtggggggaactgagttctgtgtctaattgcattacttatgtgtccttgaataggacccataactcctcccggcatgcttttgcatctatagaactgaagctgtagttctagatggcactaagggtcttggttcatattcagctgctgcttccccccaaggaaaagatgaggtttttccccttcgattaccttttgagggatgtttaggctcaaattctgcttgccacagaggaaacatttctgctgtagttgtttacctccatcctagctggcagtgtgggacacacacatacagcaagtgaaggtgggggtttgggcctgtatagaggtcagctacgctgcaggctcatctattcacaccagcaggtacagagaaaactagtgttttgtaaagtcaaatatttgttgggggttggagttctggggttggaggaggggctgttctgggcatcagttgagcagatgcccaggatgcctgggggagaccagcttcccctacaaatcagagctctaaattacaaggtttttaccaacgtgaacagttgggggaagtcgtctgctctcatttgcgtaatggtttctgtcactggtgattagacacaggatgaaggaaaagaaatttgacaattaggaatgagcgatcattaatctgaatctgttaggagacagagaggggaaggatccttatcagtgggccagcccagtatggggagacactccctccctctgcccccagagactcctgggctacatcctctttcagtattgccacaagtgggcagagcttgtatttcttaacaaagattagggacccagttgccagcctgagatggatataggaacagacatctttgggcatgagccaacaaagatagaaatagatgagtgtcaggtacaagctataaggcaaagaataacagtggaaaagttttgacatgtgtttgccatttgtggaaaatgtttttgtaatagtcaacacccctcatagcccacctgtgacaagtacttgttacattccagcactgtagggcgtgaggaaaatctggtccttatcaaatcccaggagcttctgcttagttggggaagaaattacatgaagcaaccagaggttataaggccacacttgtatatcgtgcaccctgtgtggacaagattagggactgttgagagaggaggaaaccagtagagagcaaagctctacccaggctccttgtaagcctctgggctcccccgagagggcctcgctactctacgcttcctagcaacgttgatgtccccacaaccccacatcagtgcagctgtggctgtgtggaggggctctgaggcctctgaggccagatgtgtaaacagtgctgaggttcagtaataggatgagtcttcaggtgtggagcagcccaccttggctcttcccatgtctctgtgttacttctcatattctgctgtcctttcaaacttcaaggacagtattaatttatactagtatttcttcctcagttttgtgacttgaatgcagtgagtgccttagaggatccaaggatgaaggaatgcgggttggtggttctctctttcagaatgggaacttcccaaaaatggggctgcgtctcgcctctcagtaggttccctacctctgggtcttccacccttcaaaatctggtacagaatttagcaggggctgcagggaatgaccctcagggaccagtttcacccagatggggtagatgatgaggtatacctagaagtagctcagggctccagagtctggcctttcagctggtgtcttactcccctttgagaccttcagctgcttgtcttccactgcctgcatacttctctgatgtcttctcaaactgttggctctaaagggatgttccagcctccctctccagagctgcctgcagtttcccttattcagctgtctcctgcctagtctctccagcggcctccttaccaagaccctttttcctaaacagttgtccttccctcagaaactccatggctgcaagtgtgaatagacttgtctttaggatccctctctctggtcatccctgatcatcttagggccgtctcccttttctgatttttgacagctctgtttattttgcccatggctagtcctcttgttttgtgtctgctgtgtgttcttgcccttccattgtcagttctgtacatcctggttcagcttgtctcactgtggtagccccttctgcttgtcttcagccatcttggtttaatcctcataggtcatggatgaatggagccttgctgagcactttgagggcatttctgttgggcacaacctcagaaacgggaagtcttgaggcctgggggcgtgtggtcagtgaggcctccagaggtgggttaggggtgggcacctacaagggccagttgaggggagaggtgcagggattgctgaggagactttgtgagaatgatgtcaggcccgggaagctggtgtggaagaactgagctgtcagaaggagataccaatatcccctttgcagggtggtgatgggcatcaaacatgacgtggaacatgtccccttcatggagggtttccatctaaatgcatctgtgtgccttagggatcagtagttcaggggtggtggacttttttgagtgtctcctaatttctctgaggtgtctttggctctgtgtcaccaaatgtgcctgacctctgcatcccagtggttgctgaaagggaaacagtatattgcgtagtcagatgtcaggagtagagatcttaatgagaaaggccctggcagctggagagatgagaggacctagtggtcagcttatacccagactatctctccactcctccaagagtttgagtcccatagctgatgaaagaatttacatatgggctcccccctgctttcccactgaaaatctattctcatattttgggaaggagcatatgtcctttaactttcaatggtaaacaatctaggatagtgtttaaaggcatggattttggagtcacatatagttggtttgaatttctagcttcactacttaccagctgggtttgaccttggttgatttacttaacctctctgagcctcagtttcctcaactataataacatgggtggagggaggcatgttgaaaatatctacctcagggttgttggaataactgaaacaatgtttgtaaagctttagcacagtgcctggcaagcacttaataaatggctgtggtggtggtggttatatttaaaaaaaaaaaaaaaaa

SEQ ID NO: 22 - protein, 349 aa, Homo sapiens
MAATAAEAVASGSGEPREEAGALGPAWDESQLRSYSFPTRPIPRLSQSDPRAEELIENEEPVVLTDTNLVYPALKWDLEYLQENIGNGDFSVYSASTHKFLYYDEKKMANFQNFKPRSNREEMKFHEFVEKLQDIQQRGGEERLYLQQTLNDTVGRKIVMDFLGFNWNWINKQQGKRGWGQLTSNLLLIGMEGNVTPAHYDEQQNFFAQIKGYKRCILFPPDQFECLYPYPVHHPCDRQSQVDFDNPDYERFPNFQNVVGYETVVGPGDVLYIPMYWWHHIESLLNGGITITVNFWYKGAPTPKRIEYPLKAHQKVAIMRNIEKMLGEALGNPQEVGPLLNTMIKGRYN

SEQ ID NO: 23 - protein, 241 aa, Artificial Sequence
  Synthetic
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPPGPPGPPGPPGPPG

SEQ ID NO: 24 - protein, 15 aa, Artificial Sequence
  Synthetic
PPGPPGPPGPPGPPG

SEQ ID NO: 25 - DNA, 113 bp, Artificial Sequence
  Synthetic
gctaggatccccgccgggtccgccaggcccaccgggtccacctggcccgcctggttaaaggagaagcaggtgctggacagggcgaggcatcgtgcctggttaactcgagctag

SEQ ID NO: 26 - DNA, 20 bp, Artificial Sequence
  Synthetic
gctaggatccccgccgggtc

SEQ ID NO: 27 - DNA, 20 bp, Artificial Sequence
  Synthetic
ctagctcgagttaaccaggc

SEQ ID NO: 28 - DNA, 48 bp, Artificial Sequence
  Synthetic
cagctaccatgggttcccctatactaggttattggaaaattaagggcc

SEQ ID NO: 29 - DNA, 22 bp, Artificial Sequence
  Synthetic
cctgacgggcttgtctgctccc

SEQ ID NO: 30 - protein, 4 aa, Artificial Sequence
  Synthetic
GFPG

SEQ ID NO: 31 - protein, 224 aa, Artificial Sequence
  Synthetic
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPR

SEQ ID NO: 32 - protein, 17 aa, Artificial Sequence
  Synthetic
GSPPGPPGPPGPPGPPG

SEQ ID NO: 33 - DNA, 22 bp, Artificial Sequence
  Synthetic
tttcaccatatgtctactatcc

SEQ ID NO: 34 - DNA, 22 bp, Artificial Sequence
  Synthetic
aaggatcctagtcggacaactc

SEQ ID NO: 35 - DNA, 28 bp, Artificial Sequence
  Synthetic
cccgaaaggaagctcgagttggctgctg

SEQ ID NO: 36 - DNA, 28 bp, Artificial Sequence
  Synthetic
cagcagccaactcgagcttcctttcggg

SEQ ID NO: 37 - DNA, 31 bp, Artificial Sequence
  Synthetic
cgtgcctggttaactgagcggccgcataatg

SEQ ID NO: 38 - DNA, 31 bp, Artificial Sequence
  Synthetic
cattatgcggccgctcagttaaccaggcacg

SEQ ID NO: 39 - protein, 17 aa, Artificial Sequence
  Synthetic
GSPPGPPGPPGPPGPPG

SEQ ID NO: 40 - DNA, 5927 bp, Homo sapiens
  CDS (127)..(4521)
tcgtcggagcagacgggagtttctcctcggggtcggagcaggaggcacgcggagtgtgaggccacgcatgagcggacgctaaccccctccccagccacaaagagtctacatgtctagggtctagacatgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccctgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgactggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccctggtcccatgggcccccctggattggctggaccccctggtgaatctggacgtgagggggctcctggtgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcctgtcggccctgttggcgcccgtggccccgccggaccccaaggcccccgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctccagggctccaacgagatcgagatccgcgccgagggcaacagccgcttcacctacagcgtcactgtcgatggctgcacgagtcacaccggagcctggggcaagacagtgattgaatacaaaaccaccaagacctcccgcctgcccatcatcgatgtggcccccttggacgttggtgccccagaccaggaattcggcttcgacgttggccctgtctgcttcctgtaaactccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaagaataaataaataactttttaaaaaaggaagcttggtccacttgcttgaagacccatgcgggggtaagtccctttctgcccgttgggcttatgaaaccccaatgctgccctttctgctcctttctccacaccccccttggggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctgggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcaatcccttgtgccgcagggctgggcgggagagactgttctgttccttgtgtaactgtgttgctgaaagactacctcgttcttgtcttgatgtgtcaccggggcaactgcctgggggcggggatgggggcagggtggaagcggctccccattttataccaaaggtgctacatctatgtgatgggtggggtggggagggaatcactggtgctatagaaattgagatgcccccccaggccagcaaatgttcctttttgttcaaagtctatttttattccttgatatttttctttttttttttttttttttgtggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccccctaggggtgggaggaagcaaaagactctgtacctattttgtatgtgtataataatttgagatgtttttaattattttgattgctggaataaagcatgtggaaatgacccaaacataa

SEQ ID NO: 41 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 42 - DNA, 5411 bp, Homo sapiens
  CDS (472)..(4572)
gtgtcccatagtgtttccaaacttggaaagggcgggggagggcgggaggatgcggagggcggaggtatgcagacaacgagtcagagtttccccttgaaagcctcaaaagtgtccacgtcctcaaaaagaatggaaccaatttaagaagccagccccgtggccacgtcccttcccccattcgctccctcctctgcgcccccgcaggctcctcccagctgtggctgcccgggcccccagccccagccctcccattggtggaggcccttttggaggcaccctagggccagggaaacttttgccgtataaatagggcagatccgggctttattattttagcaccacggcagcaggaggtttcggctaagttggaggtactggccacgactgcatgcccgcgcccgccaggtgatacctccgccggtgacccaggggctctgcgacacaaggagtctgcatgtctaagtgctagacatgctcagctttgtggatacgcggactttgttgctgcttgcagtaaccttatgcctagcaacatgccaatctttacaagaggaaactgtaagaaagggcccagccggagatagaggaccacgtggagaaaggggtccaccaggccccccaggcagagatggtgaagatggtcccacaggccctcctggtccacctggtcctcctggcccccctggtctcggtgggaactttgctgctcagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtccccctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcctggtgctgtaggtgcccctggtcctgctggagccacaggtgaccggggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctaccaggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgatggagacttctacagggctgaccagcctcgctcagcaccttctctcagacccaaggactatgaagttgatgctactctgaagtctctcaacaaccagattgagacccttcttactcctgaaggctctagaaagaacccagctcgcacatgccgtgacttgagactcagccacccagagtggagcagtggttactactggattgaccctaaccaaggatgcactatggatgctatcaaagtatactgtgatttctctactggcgaaacctgtatccgggcccaacctgaaaacatcccagccaagaactggtataggagctccaaggacaagaaacacgtctggctaggagaaactatcaatgctggcagccagtttgaatataatgtagaaggagtgacttccaaggaaatggctacccaacttgccttcatgcgcctgctggccaactatgcctctcagaacatcacctaccactgcaagaacagcattgcatacatggatgaggagactggcaacctgaaaaaggctgtcattctacagggctctaatgatgttgaacttgttgctgagggcaacagcaggttcacttacactgttcttgtagatggctgctctaaaaagacaaatgaatggggaaagacaatcattgaatacaaaacaaataagccatcacgcctgcccttccttgatattgcacctttggacatcggtggtgctgaccaggaattctttgtggacattggcccagtctgtttcaaataaatgaactcaatctaaattaaaaaagaaagaaatttgaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttcccccgctcccccaaaaatttgaatttttttttcaacactcttacacctgttatggaaaatgtcaacctttgtaagaaaaccaaaataaaaattgaaaaataaaaaccataaacatttgcaccacttgtggcttttgaatatcttccacagagggaagtttaaaacccaaacttccaaaggtttaaactacctcaaaacactttcccatgagtgtgatccacattgttaggtgctgacctagacagagatgaactgaggtccttgttttgttttgttcataatacaaaggtgctaattaatagtatttcagatacttgaagaatgttgatggtgctagaagaatttgagaagaaatactcctgtattgagttgtatcgtgtggtgtattttttaaaaaatttgatttagcattcatattttccatcttattcccaattaaaagtatgcagattatttgcccaaatcttcttcagattcagcatttgttctttgccagtctcattttcatcttcttccatggttccacagaagctttgtttcttgggcaagcagaaaaattaaattgtacctattttgtatatgtgagatgtttaaataaattgtgaaaaaaatgaaataaagcatgtttggttttccaaaagaacatat

SEQ ID NO: 43 - protein, 1366 aa, Homo sapiens
MLSFVDTRTLLLLAVTLCLATCQSLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPPGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGLKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMDAIKVYCDFSTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADQEFFVDIGPVCFK

SEQ ID NO: 44 - DNA, 5087 bp, Homo sapiens
  CDS (182)..(4645)
aacgggcgccgcggcggggagaagacgcagagcgctgctgggctgccgggtctcccgcttccccctcctgctccaagggcctcctgcatgagggcgcggtagagacccggacccgcgccgtgctcctgccgtttcgctgcgctccgcccgggcccggctcagccaggccccgcggtgagccatgattcgcctcggggctccccagacgctggtgctgctgacgctgctcgtcgccgctgtccttcggtgtcagggccaggatgtccaggaggctggcagctgtgtgcaggatgggcagaggtataatgataaggatgtgtggaagccggagccctgccggatctgtgtctgtgacactgggactgtcctctgcgacgacataatctgtgaagacgtgaaagactgcctcagccctgagatccccttcggagagtgctgccccatctgcccaactgacctcgccactgccagtgggcaaccaggaccaaagggacagaaaggagaacctggagacatcaaggatattgtaggacccaaaggacctcctgggcctcagggacctgcaggggaacaaggacccagaggggatcgtggtgacaaaggtgaaaaaggtgcccctggacctcgtggcagagatggagaacctgggacccctggaaatcctggcccccctggtcctcccggcccccctggtccccctggtcttggtggaaactttgctgcccagatggctggaggatttgatgaaaaggctggtggcgcccagttgggagtaatgcaaggaccaatgggccccatgggacctcgaggacctccaggccctgcaggtgctcctgggcctcaaggatttcaaggcaatcctggtgaacctggtgaacctggtgtctctggtcccatgggtccccgtggtcctcctggtccccctggaaagcctggtgatgatggtgaagctggaaaacctggaaaagctggtgaaaggggtccgcctggtcctcagggtgctcgtggtttcccaggaaccccaggccttcctggtgtcaaaggtcacagaggttatccaggcctggacggtgctaagggagaggcgggtgctcctggtgtgaagggtgagagtggttccccgggtgagaacggatctccgggcccaatgggtcctcgtggcctgcctggtgaaagaggacggactggccctgctggcgctgcgggtgcccgaggcaacgatggtcagccaggccccgcagggcctccgggtcctgtcggtcctgctggtggtcctggcttccctggtgctcctggagccaagggtgaagccggccccactggtgcccgtggtcctgaaggtgctcaaggtcctcgcggtgaacctggtactcctgggtcccctgggcctgctggtgcctccggtaaccctggaacagatggaattcctggagccaaaggatctgctggtgctcctggcattgctggtgctcctggcttccctgggccacggggccctcctggccctcaaggtgcaactggtcctctgggcccgaaaggtcagacgggtgaacctggtattgctggcttcaaaggtgaacaaggccccaagggagaacctggccctgctggcccccagggagcccctggacccgctggtgaagaaggcaagagaggtgcccgtggagagcctggtggcgttgggcccatcggtccccctggagaaagaggtgctcccggcaaccgcggtttcccaggtcaagatggtctggcaggtcccaagggagcccctggagagcgagggcccagtggtcttgctggccccaagggagccaacggtgaccctggccgtcctggagaacctggccttcctggagcccggggtctcactggccgccctggtgatgctggtcctcaaggcaaagttggcccttctggagcccctggtgaagatggtcgtcctggacctccaggtcctcagggggctcgtgggcagcctggtgtcatgggtttccctggccccaaaggtgccaacggtgagcctggcaaagctggtgagaagggactgcctggtgctcctggtctgaggggtcttcctggcaaagatggtgagacaggtgctgcaggaccccctggccctgctggacctgctggtgaacgaggcgagcagggtgctcctgggccatctgggttccagggacttcctggccctcctggtcccccaggtgaaggtggaaaaccaggtgaccagggtgttcccggtgaagctggagcccctggcctcgtgggtcccaggggtgaacgaggtttcccaggtgaacgtggctctcccggtgcccagggcctccagggtccccgtggcctccccggcactcctggcactgatggtcccaaaggtgcatctggcccagcaggcccccctggggctcagggccctccaggtcttcagggaatgcctggcgagaggggagcagctggtatcgctgggcccaaaggcgacaggggtgacgttggtgagaaaggccctgagggagcccctggaaaggatggtggacgaggcctgacaggtcccattggcccccctggcccagctggtgctaatggcgagaagggagaagttggacctcctggtcctgcaggaagtgctggtgctcgtggcgctccgggtgaacgtggagagactgggccccccggaccagcgggatttgctgggcctcctggtgctgatggccagcctggggccaagggtgagcaaggagaggccggccagaaaggcgatgctggtgcccctggtcctcagggcccctctggagcacctgggcctcagggtcctactggagtgactggtcctaaaggagcccgaggtgcccaaggccccccgggagccactggattccctggagctgctggccgcgttggacccccaggctccaatggcaaccctggaccccctggtccccctggtccttctggaaaagatggtcccaaaggtgctcgaggagacagcggcccccctggccgagctggtgaacccggcctccaaggtcctgctggaccccctggcgagaagggagagcctggagatgacggtccctctggtgccgaaggtccaccaggtccccagggtctggctggtcagagaggcatcgtcggtctgcctgggcaacgtggtgagagaggattccctggcttgcctggcccgtcgggtgagcccggcaagcagggtgctcctggagcatctggagacagaggtcctcctggccccgtgggtcctcctggcctgacgggtcctgcaggtgaacctggacgagagggaagccccggtgctgatggcccccctggcagagatggcgctgctggagtcaagggtgatcgtggtgagactggtgctgtgggagctcctggagcccctgggccccctggctcccctggccccgctggtccaactggcaagcaaggagacagaggagaagctggtgcacaaggccccatgggaccctcaggaccagctggagcccggggaatccagggtcctcaaggccccagaggtgacaaaggagaggctggagagcctggcgagagaggcctgaagggacaccgtggcttcactggtctgcagggtctgcccggccctcctggtccttctggagaccaaggtgcttctggtcctgctggtccttctggccctagaggtcctcctggccccgtcggtccctctggcaaagatggtgctaatggaatccctggccccattgggcctcctggtccccgtggacgatcaggcgaaaccggccctgctggtcctcctggaaatcctggaccccctggtcctccaggtccccctggccctggcatcgacatgtccgcctttgctggcttaggcccgagagagaagggccccgaccccctgcagtacatgcgggccgaccaggcagccggtggcctgagacagcatgacgccgaggtggatgccacactcaagtccctcaacaaccagattgagagcatccgcagccccgagggctcccgcaagaaccctgctcgcacctgcagagacctgaaactctgccaccctgagtggaagagtggagactactggattgaccccaaccaaggctgcaccttggacgccatgaaggttttctgcaacatggagactggcgagacttgcgtctaccccaatccagcaaacgttcccaagaagaactggtggagcagcaagagcaaggagaagaaacacatctggtttggagaaaccatcaatggtggcttccatttcagctatggagatgacaatctggctcccaacactgccaacgtccagatgaccttcctacgcctgctgtccacggaaggctcccagaacatcacctaccactgcaagaacagcattgcctatctggacgaagcagctggcaacctcaagaaggccctgctcatccagggctccaatgacgtggagatccgggcagagggcaatagcaggttcacgtacactgccctgaaggatggctgcacgaaacataccggtaagtggggcaagactgttatcgagtaccggtcacagaagacctcacgcctccccatcattgacattgcacccatggacataggagggcccgagcaggaattcggtgtggacatagggccggtctgcttcttgtaaaaacctgaacccagaaacaacacaatccgttgcaaacccaaaggacccaagtactttccaatctcagtcactctaggactctgcactgaatggctgacctgacctgatgtccattcatcccaccctctcacagttcggacttttctcccctctctttctaagagacctgaactgggcagactgcaaaataaaatctcggtgttctatttatttattgtcttcctgtaagaccttcgggtcaaggcagaggcaggaaactaactggtgtgagtcaaatgccccctgagtgactgcccccagcccaggccagaagacctcccttcaggtgccgggcgcaggaactgtgtgtgtcctacacaatggtgctattctgtgtcaaacacctctgtattttttaaaacatcaattgatattaaaaatgaaaagattattggaaagtaca

SEQ ID NO: 45 - protein, 1487 aa, Homo sapiens
MIRLGAPQTLVLLTLLVAAVLRCQGQDVQEAGSCVQDGQRYNDKDVWKPEPCRICVCDTGTVLCDDIICEDVKDCLSPEIPFGECCPICPTDLATASGQPGPKGQKGEPGDIKDIVGPKGPPGPQGPAGEQGPRGDRGDKGEKGAPGPRGRDGEPGTPGNPGPPGPPGPPGPPGLGGNFAAQMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRADQAAGGLRQHDAEVDATLKSLNNQIESIRSPEGSRKNPARTCRDLKLCHPEWKSGDYWIDPNQGCTLDAMKVFCNMETGETCVYPNPANVPKKNWWSSKSKEKKHIWFGETINGGFHFSYGDDNLAPNTANVQMTFLRLLSTEGSQNITYHCKNSIAYLDEAAGNLKKALLIQGSNDVEIRAEGNSRFTYTALKDGCTKHTGKWGKTVIEYRSQKTSRLPIIDIAPMDIGGPEQEFGVDIGPVCFL

SEQ ID NO: 46 - DNA, 5490 bp, Homo sapiens
  CDS (118)..(4518)
ggctgagttttatgacgggcccggtgctgaagggcagggaacaacttgatggtgctactttgaactgcttttcttttctcctttttgcacaaagagtctcatgtctgatatttagacatgatgagctttgtgcaaaaggggagctggctacttctcgctctgcttcatcccactattattttggcacaacaggaagctgttgaaggaggatgttcccatcttggtcagtcctatgcggatagagatgtctggaagccagaaccatgccaaatatgtgtctgtgactcaggatccgttctctgcgatgacataatatgtgacgatcaagaattagactgccccaacccagaaattccatttggagaatgttgtgcagtttgcccacagcctccaactgctcctactcgccctcctaatggtcaaggacctcaaggccccaagggagatccaggccctcctggtattcctgggagaaatggtgaccctggtattccaggacaaccagggtcccctggttctcctggcccccctggaatctgtgaatcatgccctactggtcctcagaactattctccccagtatgattcatatgatgtcaagtctggagtagcagtaggaggactcgcaggctatcctggaccagctggccccccaggccctcccggtccccctggtacatctggtcatcctggttcccctggatctccaggataccaaggaccccctggtgaacctgggcaagctggtccttcaggccctccaggacctcctggtgctataggtccatctggtcctgctggaaaagatggagaatcaggtagacccggacgacctggagagcgaggattgcctggacctccaggtatcaaaggtccagctgggatacctggattccctggtatgaaaggacacagaggcttcgatggacgaaatggagaaaagggtgaaacaggtgctcctggattaaagggtgaaaatggtcttccaggcgaaaatggagctcctggacccatgggtccaagaggggctcctggtgagcgaggacggccaggacttcctggggctgcaggtgctcggggtaatgacggtgctcgaggcagtgatggtcaaccaggccctcctggtcctcctggaactgccggattccctggatcccctggtgctaagggtgaagttggacctgcagggtctcctggttcaaatggtgcccctggacaaagaggagaacctggacctcagggacacgctggtgctcaaggtcctcctggccctcctgggattaatggtagtcctggtggtaaaggcgaaatgggtcccgctggcattcctggagctcctggactgatgggagcccggggtcctccaggaccagccggtgctaatggtgctcctggactgcgaggtggtgcaggtgagcctggtaagaatggtgccaaaggagagcccggaccacgtggtgaacgcggtgaggctggtattccaggtgttccaggagctaaaggcgaagatggcaaggatggatcacctggagaacctggtgcaaatgggcttccaggagctgcaggagaaaggggtgcccctgggttccgaggacctgctggaccaaatggcatcccaggagaaaagggtcctgctggagagcgtggtgctccaggccctgcagggcccagaggagctgctggagaacctggcagagatggcgtccctggaggtccaggaatgaggggcatgcccggaagtccaggaggaccaggaagtgatgggaaaccagggcctcccggaagtcaaggagaaagtggtcgaccaggtcctcctgggccatctggtccccgaggtcagcctggtgtcatgggcttccccggtcctaaaggaaatgatggtgctcctggtaagaatggagaacgaggtggccctggaggacctggccctcagggtcctcctggaaagaatggtgaaactggacctcagggacccccagggcctactgggcctggtggtgacaaaggagacacaggaccccctggtccacaaggattacaaggcttgcctggtacaggtggtcctccaggagaaaatggaaaacctggggaaccaggtccaaagggtgatgccggtgcacctggagctccaggaggcaagggtgatgctggtgcccctggtgaacgtggacctcctggattggcaggggccccaggacttagaggtggagctggtccccctggtcccgaaggaggaaagggtgctgctggtcctcctgggccacctggtgctgctggtactcctggtctgcaaggaatgcctggagaaagaggaggtcttggaagtcctggtccaaagggtgacaagggtgaaccaggcggtccaggtgctgatggtgtcccagggaaagatggcccaaggggtcctactggtcctattggtcctcctggcccagctggccagcctggagataagggtgaaggtggtgcccccggacttccaggtatagctggacctcgtggtagccctggtgagagaggtgaaactggccctccaggacctgctggtttccctggtgctcctggacagaatggtgaacctggtggtaaaggagaaagaggggctccgggtgagaaaggtgaaggaggccctcctggagttgcaggaccccctggaggttctggacctgctggtcctcctggtccccaaggtgtcaaaggtgaacgtggcagtcctggtggacctggtgctgctggcttccctggtgctcgtggtcttcctggtcctcctggtagtaatggtaacccaggacccccaggtcccagcggttctccaggcaaggatgggcccccaggtcctgcgggtaacactggtgctcctggcagccctggagtgtctggaccaaaaggtgatgctggccaaccaggagagaagggatcgcctggtgcccagggcccaccaggagctccaggcccacttgggattgctgggatcactggagcacggggtcttgcaggaccaccaggcatgccaggtcctaggggaagccctggccctcagggtgtcaagggtgaaagtgggaaaccaggagctaacggtctcagtggagaacgtggtccccctggaccccagggtcttcctggtctggctggtacagctggtgaacctggaagagatggaaaccctggatcagatggtcttccaggccgagatggatctcctggtggcaagggtgatcgtggtgaaaatggctctcctggtgcccctggcgctcctggtcatccaggcccacctggtcctgtcggtccagctggaaagagtggtgacagaggagaaagtggccctgctggccctgctggtgctcccggtcctgctggttcccgaggtgctcctggtcctcaaggcccacgtggtgacaaaggtgaaacaggtgaacgtggagctgctggcatcaaaggacatcgaggattccctggtaatccaggtgccccaggttctccaggccctgctggtcagcagggtgcaatcggcagtccaggacctgcaggccccagaggacctgttggacccagtggacctcctggcaaagatggaaccagtggacatccaggtcccattggaccaccagggcctcgaggtaacagaggtgaaagaggatctgagggctccccaggccacccagggcaaccaggccctcctggacctcctggtgcccctggtccttgctgtggtggtgttggagccgctgccattgctgggattggaggtgaaaaagctggcggttttgccccgtattatggagatgaaccaatggatttcaaaatcaacaccgatgagattatgacttcactcaagtctgttaatggacaaatagaaagcctcattagtcctgatggttctcgtaaaaaccccgctagaaactgcagagacctgaaattctgccatcctgaactcaagagtggagaatactgggttgaccctaaccaaggatgcaaattggatgctatcaaggtattctgtaatatggaaactggggaaacatgcataagtgccaatcctttgaatgttccacggaaacactggtggacagattctagtgctgagaagaaacacgtttggtttggagagtccatggatggtggttttcagtttagctacggcaatcctgaacttcctgaagatgtccttgatgtgcagctggcattccttcgacttctctccagccgagcttcccagaacatcacatatcactgcaaaaatagcattgcatacatggatcaggccagtggaaatgtaaagaaggccctgaagctgatggggtcaaatgaaggtgaattcaaggctgaaggaaatagcaaattcacctacacagttctggaggatggttgcacgaaacacactggggaatggagcaaaacagtctttgaatatcgaacacgcaaggctgtgagactacctattgtagatattgcaccctatgacattggtggtcctgatcaagaatttggtgtggacgttggccctgtttgctttttataaaccaaactctatctgaaatcccaacaaaaaaaatttaactccatatgtgttcctcttgttctaatcttgtcaaccagtgcaagtgaccgacaaaattccagttatttatttccaaaatgtttggaaacagtataatttgacaaagaaaaatgatacttctctttttttgctgttccaccaaatacaattcaaatgctttttgttttatttttttaccaattccaatttcaaaatgtctcaatggtgctataataaataaacttcaacactctttatgataacaacactgtgttatattctttgaatcctagcccatctgcagagcaatgactgtgctcaccagtaaaagataacctttctttctgaaatagtcaaatacgaaattagaaaagccctccctattttaactacctcaactggtcagaaacacagattgtattctatgagtcccagaagatgaaaaaaattttatacgttgataaaacttataaatttcattgattaatctcctggaagattggtttaaaaagaaaagtgtaatgcaagaatttaaagaaatatttttaaagccacaattattttaatattggatatcaactgcttgtaaaggtgctcctcttttttcttgtcattgctggtcaagattactaatatttgggaaggctttaaagacgcatgttatggtgctaatgtactttcacttttaaactctagatcagaattgttgacttgcattcagaacataaatgcacaaaatctgtacatgtctcccatcagaaagattcattggcatgccacaggggattctcctccttcatcctgtaaaggtcaacaataaaaaccaaattatggggctgcttttgtcacactagcatagagaatgtgttgaaatttaactttgtaagcttgtatgtggttgttgatcttttttttccttacagacacccataataaaatatcatattaaaattc

SEQ ID NO: 47 - protein, 1466 aa, Homo sapiens
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 48 - DNA, 8439 bp, Homo sapiens
  CDS (383)..(5899)
cgcactctccgtccccgcggctggcgcaggacctcactcgagcggagcgcccacggggagcgggtcgcggggcggcggcggcgaggaggaggcgagaaggagttggaggaggaggaggaggaggcgagggcgagctagcccagcggggtcccggccgccccgcgggccaaagtcgagccctcccgcccgtgggcgagcgcgccagccgccccttccagaacagccgccgccacaaagaagaacggggggtgccgaggtccccatgacctcctaaagtggtgcggtccctgctgagtgcgctgcccgggccgtgacccgcgcccctgtgcgtccccgcgcgcctccgagcgcccctgtgcgccccggcccgcgccccgccggcatggacgtccatacccgctggaaagcgcgcagcgcgctccgcccgggcgccccgctgctgcccccgctgctgctgctgctgctgtgggcgccgcctccgagccgcgcagctcagccagcagatctcctgaaggttctagattttcacaacttgcctgatggaataacaaagacaacaggcttttgcgccacgcggcgatcttccaaaggcccggatgtcgcttacagagtcaccaaagacgcgcagctcagcgcacccaccaagcagctgtaccctgcgtctgcatttcccgaggacttctccatcctaacaactgtgaaagccaagaaaggcagccaggccttcctggtctccatctacaacgagcagggtatccagcagattgggctggagctgggccgctctcccgtcttcctctacgaggaccacacggggaagcctggcccggaagactaccccctcttccggggcatcaacctgtcagatggcaagtggcacagaattgctctcagcgtccacaagaaaaatgtcaccttgatcctcgactgtaaaaagaagaccaccaaattcctcgaccgcagcgaccaccccatgatcgacatcaatggcatcatcgtgtttggcacccggatcctggatgaggaggtgtttgagggtgacatccagcagctgctctttgtctcggaccaccgggcagcttatgattactgtgagcactacagccctgactgtgacaccgcagtacctgacaccccacagtcgcaggaccccaatccagatgaatattacacggaaggagacggcgagggtgagacctattactacgaatacccctactacgaagaccccgaagacctagggaaggagcccacccccagcaagaagcccgtggaagctgccaaagaaaccacagaggtccccgaggagctgaccccgacccccacggaagctgctcccatgcctgaaaccagtgaaggggctgggaaggaagaggacgtcggcatcggggactatgactacgtgcccagtgaggactactacacgccctcaccgtatgatgacctcacctatggcgagggggaggagaaccccgaccagcccacagacccaggcgctggggccgaaattcccaccagcaccgccgacacctccaactcctccaatccagctccgcctccaggggaaggtgcggatgacttggagggggagttcactgaggaaacgatccggaaccttgacgagaactactacgacccctactacgaccccaccagctccccgtcggagatcgggccgggaatgccggcgaaccaggataccatctatgaagggattggaggacctcggggcgagaaaggccaaaagggagaaccagcgattatcgagccgggcatgctcatcgagggcccgcctggcccagaaggccccgcgggtcttcccggacctccaggaaccatgggtcccactggccaagtcggggaccctggagaaaggggcccccctggacgcccaggccttcctggggccgatggcctgcccggtcctccaggaaccatgctcatgctgcccttccggtttggaggtggcggcgatgcgggctccaaaggccccatggtctcagcccaggagtcccaggcgcaagccattctccagcaggccaggttggcactgaggggaccagctggcccgatgggtctcacagggagacctggccctgtgggtccccctgggagcggaggtttgaagggcgagccgggagacgtggggcctcagggtcctcgaggtgtgcaaggcccgcctggtccggccgggaagcccggaagacggggtcgggctgggagtgatggagccagaggaatgcctggacaaactggccccaagggtgaccggggtttcgacggcctggctgggttgccaggcgagaagggccacaggggtgaccctggtccttccggcccaccaggacctccgggagacgatggagaaaggggtgacgacggagaagttgggcccagggggctgcctggggagcccgggccacgtggtctgcttgggccgaaggggcccccaggtcctcccggacctcccggtgtcacgggtatggacggccagccggggccaaaaggaaatgtgggtccccagggagagcctggccccccaggacagcagggtaatccaggcgcccagggtcttccaggcccccagggtgcaattggtcctccaggagaaaagggtcccttggggaaaccaggccttccaggaatgcccggtgctgacggacccccgggacaccctggcaaagaaggccctccaggagagaaaggaggtcagggtccacctggcccccagggtccgattggctacccaggtcctcgaggagtcaagggggccgatggcatccgtggtctgaagggcacaaagggcgagaagggtgaagacggctttcctgggtttaaaggagacatgggcatcaagggtgatcggggggagatcggcccacccggtcccaggggagaagatggccctgaaggcccaaagggtcgcggaggtcccaatggtgaccccggtcctctgggaccccctggggagaagggaaaactcggagtcccagggttaccagggtatccaggaagacaaggaccaaagggctctattggattccctggatttcctggcgccaatggagagaagggcggcagggggacccctggaaagccaggaccgcgggggcagcgaggcccaacgggtccgaggggtgaaagaggcccccggggcatcactgggaagcctggccccaagggcaactccggaggtgacggcccagctggccctcctggtgaacggggacccaatggaccccaaggacccacaggatttcctggaccaaagggcccccctggccctccaggcaaggatggactcccaggacaccctggacagagaggcgagactggtttccaaggcaagaccggccctccaggcccccccggcgtggtcggccctcagggtcccacgggagaaacgggcccaatgggtgagcgtggccaccctgggccccctggaccccccggtgaacaggggcttccgggccttgctggaaaagaagggacgaagggtgacccaggccctgcaggcctccctgggaaagatggccctccaggattacgtggtttccctggggaccgagggcttcctggtccagtgggagctcttggactgaaaggcaatgaagggccccctggcccaccaggccctgcgggatctccaggggagagaggtccagctggagccgctgggcccatcggaattccagggagacctgggccccagggacccccagggccggcaggagagaaaggggctcctggcgagaaaggcccacaaggcccagctggccgagacggtctccaggggcctgtggggctcccgggtccagctggccctgtgggtccccctggagaagacggagataagggagagatcggggagccggggcagaaaggaagcaagggggacaaaggagaacagggtcctcctgggcctacaggtcctcaaggccccatcggacagccaggcccctctggagctgacggcgagccggggcctcggggccagcagggccttttcgggcagaaaggtgatgaaggtcccagaggctttcctggaccccctgggccagtggggctgcagggtttgccaggacctccaggcgagaagggtgagacaggagacgtgggccagatgggccccccgggtccccctggcccccgaggaccctccggagctccaggtgctgatggcccacaaggtcccccaggtggaataggaaaccctggtgcagtgggagagaagggcgagcctggcgaagcaggtgagcctggccttccgggagaaggcggccccccgggacccaaaggagaaaggggagagaagggcgagtcaggcccttcaggtgctgccggaccccctggacccaaaggccctcccggagatgatggtcccaaaggcagccctggcccagtgggttttcctggagatcctggcccccccggagagcctggccccgcgggtcaagatggtccccctggtgacaaaggagatgatggtgaacccgggcagacgggatcccccggccctactggtgaaccaggtccatcggggcctccaggaaaaaggggtcccccaggccccgcaggccccgaaggcagacagggagagaaaggggccaagggagaagccggcttggaaggccctcctgggaagactggccccatcggcccccagggggcccctgggaagcccggaccggatggccttcgagggatccctggccctgtgggagaacaaggtctcccaggatccccaggcccggacggtccccccggccccatgggtcccccaggacttcccggcctcaaaggagattctggtcccaaaggtgaaaagggtcatccaggcctgatcgggctcatcggtcctccgggtgaacagggtgagaagggcgaccgtggtctccctggcccccagggctcctccggtcctaagggagaacagggtatcactggtccttctggcccgattgggcctcctgggccccctggcctgccgggtccgcctggtccaaaaggtgctaagggctcctcgggtccaactggcccgaagggtgaggcaggccacccaggacccccaggccccccgggccccccgggagaggtcatccagcccctgccaatccaggcatccaggacgcggcggaacatcgacgccagccagctgctggacgacgggaatggcgagaactacgtggactacgcggacggcatggaagagatcttcggctctctcaactctctgaagctggagattgagcagatgaaacggcccctgggcacgcagcagaaccccgcccgcacctgcaaggacctgcagctctgccaccccgacttcccagatggtgaatactgggtcgatcctaaccaaggatgctccagggattccttcaaggtttactgcaacttcacagccggggggtcgacatgcgtcttccctgacaagaagtccgaaggggccagaatcacttcttggcccaaagaaaacccgggctcctggttcagtgaattcaagcgtgggaaactgctctcctatgtggacgccgagggcaaccctgtgggtgtggtacagatgaccttcctgcggctgctgagcgcctctgcccaccagaacgtcacctaccactgctaccagtcagtggcctggcaggacgcagccacgggcagctacgacaaggccctccgcttcctgggctccaacgacgaggagatgtcctatgacaacaacccctacatccgcgccctggtggacggctgtgctaccaagaaaggctaccagaagacggttctggagatcgacacccccaaagtggagcaggtgcccatcgtggacatcatgttcaatgacttcggtgaagcgtcacagaaatttggatttgaagtggggccggcttgcttcatgggctaggagccgccgagcccgggctcccgagagcaacctcgtgacctcagcatgccattcgttcgtgagtgtcccgtgcacgtcctgaccctggacagtgaaggcttctccctcccctcccacctgacttcatctacgcctcggcaccacggggtgtgggaccccagcccggagagaacagagggaaggagccgcgcccccacctggagctgaatcacatgacctagctgcaccccagcgcctgggcccgccccacgctctgtccacacccacgcgccccgggagcggggccatgcctccagccccccagctcgcccgacccatcctgttcgtgaataggtctcaggggttgggggagggactgccagatttggacactatatttttttctaaattcaacttgaagatgtgtatttcccctgaccttcaaaaaatgttccaaggtaagcctcgtaaaggtcatcccaccatcaccaaagcctccgtttttaacaacctccaacacgatccatttagaggccaaatgtcattctgcaggtgccttcccgatggattaaaggtgcttatgtttttgtgagttttaagtaaatatttgtattgtattgttataaatgttaagtgtgcctggctttcaatcatgcacggaaacccagtctcagtcccacggacagaatgggcgaggcatggattctgggttgcagtaccgttctgattagaaataggaagtctccccacccccgccctggccaagaacgtgcaataaattggaagtttgccccggggcagcaagaatttatgctgccattgaaaagcaggtaccagtgccccttttcagacagtttttgattcgctctagactttttttttttttaatagggaaaaaatttgataattttcttttttctacatgcacttaagactaaaacacaggtttggattaattttatttgcttcctttttccgcttttcttcccgcagagcctgatgggagaatgtccagggcagggaaaccacattttttgtaggtgataactcaatgaaaattggtgcttattttttacacttctctcttgtggctctcttgtggtgctatctatctgttttaaggtctccttgaaggcgcactggggaccctggccatgcctcgttctccctgctttctttatcctgttattgcctccacagtctgttgccaaggactctaagatcaatgcacgtcactttcctttccactgggcaggatagccaagcacactccctcctgcgctctcccgccccggtgcgtccactcccgagggctgttatgaggactgggttgtgcctacttgatttgaaaacacacacaagcaataaaaagcctcttcctgcattgtctgtggtgtgaccatagcagattatatttggttcctgaatgtttgtggtgctaatttctgtgtttgttccaagccgttcagtcatgccatgcgctgcctcggtagatggagtaatgtacaatgaactccatgagtctctccagggctgcctgcagcacgtcttttccaagtagcctatttggattcccatctcaaatgtcctggatgcgagcgtcagcggctccagagctcggggcgggtgaggtcccctttggggaaccctttcctggccatcgaggtcggggggctgccgtctgtgggcaggaggacccgaggggcagccaggaaaggcgatctcttcactgtgaaaagttgcccgggtgcagcgccttttccttctaccatgggaaatgcaggctgggcccttggggtgagcctgcggggctctggtgctgtccccgacccccaccaccaccagaatgcagttccagcttaggaagccacaaacaagccacccaggaggaacaaaacaccgccagcgtggattttccaaatttccctggaaagtaagtctcgctcttgccaaagaaaagtctggcttggagagtctctggagcccaggatgccagcatgtgccaatgactgtcaccttcatctcttcaaaagaaaagccatagccgaggactgtcccgcgacccccgtggactgcgtctaggtcatgtgattctgttttcatttctcatcccatccaatttgtccttttctcctgtcattttcttcctctgtggtcccttcaaagttgttataatttgtactgaacttcaaaatgtgtcccgttctccccagaccactctagccacagtatattgcaataaaattacttcttatatttgcagaaattcttttggtgtaattttattttttcctctcaatatatataattggacaaacgctggcaaaaagaaaaaaatggtaagcaaaaaacccaagataaagtttcgaggacatcaggccttttgaaatacaatgtcaaatgacacattgtacggtttcaaaaaatccgctagacatgtcataagttttaactgtaatgcccaggaaaggatatcttaaaatattctaaacttgtgtaacaaaggaataattaactgtaatagtttttcaataaatcgagttgggtgtttccaccgtaaa

SEQ ID NO: 49 - protein, 1838 aa, Homo sapiens
MDVHTRWKARSALRPGAPLLPPLLLLLLWAPPPSRAAQPADLLKVLDFHNLPDGITKTTGFCATRRSSKGPDVAYRVTKDAQLSAPTKQLYPASAFPEDFSILTTVKAKKGSQAFLVSIYNEQGIQQIGLELGRSPVFLYEDHTGKPGPEDYPLFRGINLSDGKWHRIALSVHKKNVTLILDCKKKTTKFLDRSDHPMIDINGIIVFGTRILDEEVFEGDIQQLLFVSDHRAAYDYCEHYSPDCDTAVPDTPQSQDPNPDEYYTEGDGEGETYYYEYPYYEDPEDLGKEPTPSKKPVEAAKETTEVPEELTPTPTEAAPMPETSEGAGKEEDVGIGDYDYVPSEDYYTPSPYDDLTYGEGEENPDQPTDPGAGAEIPTSTADTSNSSNPAPPPGEGADDLEGEFTEETIRNLDENYYDPYYDPTSSPSEIGPGMPANQDTIYEGIGGPRGEKGQKGEPAIIEPGMLIEGPPGPEGPAGLPGPPGTMGPTGQVGDPGERGPPGRPGLPGADGLPGPPGTMLMLPFRFGGGGDAGSKGPMVSAQESQAQAILQQARLALRGPAGPMGLTGRPGPVGPPGSGGLKGEPGDVGPQGPRGVQGPPGPAGKPGRRGRAGSDGARGMPGQTGPKGDRGFDGLAGLPGEKGHRGDPGPSGPPGPPGDDGERGDDGEVGPRGLPGEPGPRGLLGPKGPPGPPGPPGVTGMDGQPGPKGNVGPQGEPGPPGQQGNPGAQGLPGPQGAIGPPGEKGPLGKPGLPGMPGADGPPGHPGKEGPPGEKGGQGPPGPQGPIGYPGPRGVKGADGIRGLKGTKGEKGEDGFPGFKGDMGIKGDRGEIGPPGPRGEDGPEGPKGRGGPNGDPGPLGPPGEKGKLGVPGLPGYPGRQGPKGSIGFPGFPGANGEKGGRGTPGKPGPRGQRGPTGPRGERGPRGITGKPGPKGNSGGDGPAGPPGERGPNGPQGPTGFPGPKGPPGPPGKDGLPGHPGQRGETGFQGKTGPPGPPGVVGPQGPTGETGPMGERGHPGPPGPPGEQGLPGLAGKEGTKGDPGPAGLPGKDGPPGLRGFPGDRGLPGPVGALGLKGNEGPPGPPGPAGSPGERGPAGAAGPIGIPGRPGPQGPPGPAGEKGAPGEKGPQGPAGRDGLQGPVGLPGPAGPVGPPGEDGDKGEIGEPGQKGSKGDKGEQGPPGPTGPQGPIGQPGPSGADGEPGPRGQQGLFGQKGDEGPRGFPGPPGPVGLQGLPGPPGEKGETGDVGQMGPPGPPGPRGPSGAPGADGPQGPPGGIGNPGAVGEKGEPGEAGEPGLPGEGGPPGPKGERGEKGESGPSGAAGPPGPKGPPGDDGPKGSPGPVGFPGDPGPPGEPGPAGQDGPPGDKGDDGEPGQTGSPGPTGEPGPSGPPGKRGPPGPAGPEGRQGEKGAKGEAGLEGPPGKTGPIGPQGAPGKPGPDGLRGIPGPVGEQGLPGSPGPDGPPGPMGPPGLPGLKGDSGPKGEKGHPGLIGLIGPPGEQGEKGDRGLPGPQGSSGPKGEQGITGPSGPIGPPGPPGLPGPPGPKGAKGSSGPTGPKGEAGHPGPPGPPGPPGEVIQPLPIQASRTRRNIDASQLLDDGNGENYVDYADGMEEIFGSLNSLKLEIEQMKRPLGTQQNPARTCKDLQLCHPDFPDGEYWVDPNQGCSRDSFKVYCNFTAGGSTCVFPDKKSEGARITSWPKENPGSWFSEFKRGKLLSYVDAEGNPVGVVQMTFLRLLSASAHQNVTYHCYQSVAWQDAATGSYDKALRFLGSNDEEMSYDNNPYIRALVDGCATKKGYQKTVLEIDTPKVEQVPIVDIMFNDFGEASQKFGFEVGPACFMG

SEQ ID NO: 50 - DNA, 6930 bp, Homo sapiens
  CDS (276)..(4775)
gaccgttgcttggcagacactggatggttatgagcctgaacaagctgaaaaggggcaggaaaagaagtggaggcagcattcttcctatttaaagctgcatcgcttgaaaaaagttttcgcagactgtgctggagctggtgctgaaaaagggggtttgcagaggctgccctggggctggtgctgaaagaagagcccacagctgacttcatggtgctacaataacctcagaatctacttttcactctcaggagaacccacatgtctaatatttagacatgatggcaaactgggcggaagcaagacctctcctcattcttattgttttattagggcaatttgtctcaataaaagcccaggaagaagacgaggatgaaggatatggtgaagaaatagcctgcactcagaatggccagatgtacttaaacagggacatttggaaacctgccccttgtcagatctgtgtctgtgacaatggagccattctctgtgacaagatagaatgccaggatgtgctggactgtgccgaccctgtaacgccccctggggaatgctgtcctgtctgttcacaaacacctggaggtggcaatacaaattttggtagaggaagaaagggacaaaagggagaaccaggattagtgcctgttgtaacaggcatacgtggtcgtccaggaccggcaggacctccaggatcacagggaccaagaggagagcgagggccaaaaggaagacctggccctcgtggacctcagggaattgatggagaaccaggtgttcctggtcaacctggtgctccaggacctcctggacatccgtcccacccaggacccgatggcttgagcaggccgttttcagctcaaatggctgggttggatgaaaaatctggacttgggagtcaagtaggactaatgcctggctctgtgggtcctgttggcccaaggggaccacagggtttacaaggacagcaaggtggtgcaggacctacaggacctcctggtgaacctggtgatcctggaccaatgggtccgattggttcacgtggaccagagggccctcctggtaaacctggggaagatggtgaacctggcagaaatggaaatcctggtgaagtgggatttgcaggatctccgggagctcgtggatttcctggggctcctggtcttccaggtctgaagggtcaccgaggacacaaaggtcttgaaggccctaaaggtgaagttggagcacctggttccaagggtgaagctggccccactggtccaatgggtgccatgggtcctctgggtccgaggggaatgccaggagagagagggagacttgggccacagggtgctcctggacaacgaggtgcacatggtatgcctggaaaacctggaccaatgggtcctcttgggataccaggctcttctggttttccaggaaatcctggaatgaagggagaagcaggtcctacaggggcgcgaggccctgaaggtcctcaggggcagagaggtgaaactgggcccccaggtccagttggctctccaggtcttcctggtgcaataggaactgatggtactcctggtgccaaaggcccaacgggctctccgggtacctctggtcctcctggctcagcagggcctcctggatctccaggacctcagggtagcactggtcctcagggaattcgaggccaaccgggtgatccaggagttccaggtttcaaaggagaagctggcccaaaaggggaaccagggccacatggtattcagggtccgataggcccacccggtgaagaaggcaaaagaggtcccagaggtgacccaggaacagttggtcctccagggccagtgggagaaaggggtgctcctggcaatcgtggttttccaggctctgatggtttacctgggccaaagggtgctcaaggagaacggggtcctgtaggttcttcaggacccaaaggaagccagggggatccaggacgtccaggggaacctgggcttccaggtgctcggggtttgacaggaaatcctggtgttcaaggtcctgaaggaaaacttggacctttgggtgcgccaggggaagatggccgtccaggtcctccaggctccataggaatcagagggcagcccgggagcatgggccttccaggccccaaaggtagcagtggtgaccctgggaaacctggagaagcaggaaatgctggagttcctgggcagaggggagctcctggaaaagatggtgaagttggtccttctggtcctgtgggcccgccgggtctagctggtgaaagaggagaacaaggacctccaggccccacaggttttcaggggcttcctggtcctccagggcctcctggagaaggtggaaaaccaggtgatcaaggtgttcctggagatcccggagcagttggcccgttaggacctagaggagaacgaggaaatcctggggaaagaggagaacctgggataactggactccctggtgagaagggaatggctggaggacatggtcctgatggcccaaaaggcagtccaggtccatctgggacccctggagatacaggcccaccaggtcttcaaggtatgccgggagaaagaggaattgcaggaactcctggccccaagggtgacagaggtggcataggagaaaaaggtgctgaaggcacagctggaaatgatggtgcaagaggtcttccaggtcctttgggccctccaggtccggcaggtcctactggagaaaagggtgaacctggtcctcgaggtttagttggccctcctggctcccggggcaatcctggttctcgaggtgaaaatgggccaactggagctgttggttttgccggaccccagggtcctgacggacagcctggagtaaaaggtgaacctggagagccaggacagaagggagatgctggttctcctggaccacaaggtttagcaggatcccctggccctcatggtcctaatggtgttcctggactaaaaggtggtcgaggaacccaaggtccgcctggtgctacaggatttcctggttctgcgggcagagttggacctccaggccctgctggagctccaggacctgcgggacccctaggggaacccgggaaggagggacctccaggtcttcgtggggaccctggctctcatgggcgtgtgggagatcgaggaccagctggcccccctggtggcccaggagacaaaggggacccaggagaagatgggcaacctggtccagatggcccccctggtccagctggaacgaccgggcagagaggaattgttggcatgcctgggcaacgtggagagagaggcatgcccggcctaccaggcccagcgggaacaccaggaaaagtaggaccaactggtgcaacaggagataaaggtccacctggacctgtggggcccccaggctccaatggtcctgtaggggaacctggaccagaaggtccagctggcaatgatggtaccccaggacgggatggtgctgttggagaacgtggtgatcgtggagaccctgggcctgcaggtctgccaggctctcagggtgcccctggaactcctggccctgtgggtgctccaggagatgcaggacaaagaggagatccgggttctcggggtcctataggaccacctggtcgagctgggaaacgtggattacctggaccccaaggacctcgtggtgacaaaggtgatcatggagaccgaggcgacagaggtcagaagggccacagaggctttactggtcttcagggtcttcctggccctcctggtccaaatggtgaacaaggaagtgctggaatccctggaccatttggcccaagaggtcctccaggcccagttggtccttcaggtaaagaaggaaaccctgggccacttgggccaattggacctccaggtgtacgaggcagtgtaggagaagcaggacctgagggccctcctggtgagcctggcccacctggccctccgggtccccctggccaccttacagctgctcttggggatatcatggggcactatgatgaaagcatgccagatccacttcctgagtttactgaagatcaggcggctcctgatgacaaaaacaaaacggacccaggggttcatgctaccctgaagtcactcagtagtcagattgaaaccatgcgcagccccgatggctcgaaaaagcacccagcccgcacgtgtgatgacctaaagctttgccattccgcaaagcagagtggtgaatactggattgatcctaaccaaggatctgttgaagatgcaatcaaagtttactgcaacatggaaacaggagaaacatgtatttcagcaaacccatccagtgtaccacgtaaaacctggtgggccagtaaatctcctgacaataaacctgtttggtatggtcttgatatgaacagagggtctcagttcgcttatggagaccaccaatcacctaatacagccattactcagatgacttttttgcgccttttatcaaaagaagcctcccagaacatcacttacatctgtaaaaacagtgtaggatacatggacgatcaagctaagaacctcaaaaaagctgtggttctcaaaggggcaaatgacttagatatcaaagcagagggaaatattagattccggtatatcgttcttcaagacacttgctctaagcggaatggaaatgtgggcaagactgtctttgaatatagaacacagaatgtggcacgcttgcccatcatagatcttgctcctgtggatgttggcggcacagaccaggaattcggcgttgaaattgggccagtttgttttgtgtaaagtaagccaagacacatcgacaatgagcaccaccatcaatgaccaccgccattcacaagaactttgactgtttgaagttgatcctgagactcttgaagtaatggctgatcctgcatcagcattgtatatatggtcttaagtgcctggcctccttatccttcagaatatttattttacttacaatcctcaagttttaattgattttaaatatttttcaatacaacagtttaggtttaagatgaccaatgacaatgaccacctttgcagaaagtaaactgattgaataaataaatctccgttttcttcaatttatttcagtgtaatgaaaaagttgcttagtatttatgaggaaattcttcttcctggcaggtagcttaaagagtggggtatatagagccacaacacatgtttattttgcttggctgcagttgaaaaatagaaattagtgcccttttgtgacctctcattccaagattgtcaattaaaaatgagtttaaaatgtttaacttgtgatcgagacctacatgcatgtcttgatattgtgtaactataatagagactctttaaggagaatcttaaaaaaaaaaaaacgtttctcactgtcttaaatagaatttttaaatagtatatattcagtggcattttggagaacaaagtgaatttacttcgacttcttaaatttttgtaaaagactataagtttagacatctttctcattcaaatttaaagatatctttctcctcttgatcaatctatcaatattgatagaagtcacactagtatataccatttaatacatttacactttcttatttaagaagatattgaatgcaaaataattgacatatagaactttacaaacatatgtccaaggactctaaattgagactcttccacatgtacaatctcatcatcctgaagcctataatgaagaaaaagatctagaaactgagttgtggagctgactctaatcaaatgtgatgattggaattagaccatttggcctttgaactttcataggaaaaatgacccaacatttcttagcatgagctacctcatctctagaagctgggatggacttactattcttgtttatattttagatactgaaaggtgctatgcttctgttattattccaagactggagataggcagggctaaaaaggtattattatttttcctttaatgatggtgctaaaattcttcctataaaattccttaaaaataaagatggtttaatcactaccattgtgaaaacataactgttagacttcccgtttctgaaagaaagagcatcgttccaatgcttgttcactgttcctctgtcatactgtatctggaatgctttgtaatacttgcatgcttcttagaccagaacatgtaggtccccttgtgtctcaatactttttttttcttaattgcatttgttggctctattttaatttttttcttttaaaataaacagctgggaccatcccaaaagacaagccatgcatacaactttggtcatgtatctctgcaaagcatcaaattaaatgcacgcttttgtcatgtcagtggtttttgttttgtgaaattcctttgaccatattagatctatttcatttccaatagtgaaaaggagatgtggtggtatactttgtttgccatttgtttaaaagatacaacggataccttctatcatgtatgtactggcttataaatgaaaatctatctacaacattacccacaaaggcaacatgacaccaattatcactgcctctgcccttaaaaatgtcagagtagtattattgataaaaagggcaagcaatagatttttcatgactgaataaactgtaataataaaacatatgtctcaaagtgtatcacatatgaatttagcctaattgttttcagtttcattctcaatatttagtttacaacatcattttcccctaaactggttatattttgacctgtatatcttaaatttgagtatttatatgcctaaatacatgtgtgagttttgtttgacttccaagtccaaactataagattatataagttcatatagatgaatcagaaatatgtggtaatactattaagtcacaaacactaacaatttccaactatagaaataacagttcttatttggattttgggaatgctaccaata

SEQ ID NO: 51 - protein, 1499 aa, Homo sapiens
MMANWAEARPLLILIVLLGQFVSIKAQEEDEDEGYGEEIACTQNGQMYLNRDIWKPAPCQICVCDNGAILCDKIECQDVLDCADPVTPPGECCPVCSQTPGGGNTNFGRGRKGQKGEPGLVPVVTGIRGRPGPAGPPGSQGPRGERGPKGRPGPRGPQGIDGEPGVPGQPGAPGPPGHPSHPGPDGLSRPFSAQMAGLDEKSGLGSQVGLMPGSVGPVGPRGPQGLQGQQGGAGPTGPPGEPGDPGPMGPIGSRGPEGPPGKPGEDGEPGRNGNPGEVGFAGSPGARGFPGAPGLPGLKGHRGHKGLEGPKGEVGAPGSKGEAGPTGPMGAMGPLGPRGMPGERGRLGPQGAPGQRGAHGMPGKPGPMGPLGIPGSSGFPGNPGMKGEAGPTGARGPEGPQGQRGETGPPGPVGSPGLPGAIGTDGTPGAKGPTGSPGTSGPPGSAGPPGSPGPQGSTGPQGIRGQPGDPGVPGFKGEAGPKGEPGPHGIQGPIGPPGEEGKRGPRGDPGTVGPPGPVGERGAPGNRGFPGSDGLPGPKGAQGERGPVGSSGPKGSQGDPGRPGEPGLPGARGLTGNPGVQGPEGKLGPLGAPGEDGRPGPPGSIGIRGQPGSMGLPGPKGSSGDPGKPGEAGNAGVPGQRGAPGKDGEVGPSGPVGPPGLAGERGEQGPPGPTGFQGLPGPPGPPGEGGKPGDQGVPGDPGAVGPLGPRGERGNPGERGEPGITGLPGEKGMAGGHGPDGPKGSPGPSGTPGDTGPPGLQGMPGERGIAGTPGPKGDRGGIGEKGAEGTAGNDGARGLPGPLGPPGPAGPTGEKGEPGPRGLVGPPGSRGNPGSRGENGPTGAVGFAGPQGPDGQPGVKGEPGEPGQKGDAGSPGPQGLAGSPGPHGPNGVPGLKGGRGTQGPPGATGFPGSAGRVGPPGPAGAPGPAGPLGEPGKEGPPGLRGDPGSHGRVGDRGPAGPPGGPGDKGDPGEDGQPGPDGPPGPAGTTGQRGIVGMPGQRGERGMPGLPGPAGTPGKVGPTGATGDKGPPGPVGPPGSNGPVGEPGPEGPAGNDGTPGRDGAVGERGDRGDPGPAGLPGSQGAPGTPGPVGAPGDAGQRGDPGSRGPIGPPGRAGKRGLPGPQGPRGDKGDHGDRGDRGQKGHRGFTGLQGLPGPPGPNGEQGSAGIPGPFGPRGPPGPVGPSGKEGNPGPLGPIGPPGVRGSVGEAGPEGPPGEPGPPGPPGPPGHLTAALGDIMGHYDESMPDPLPEFTEDQAAPDDKNKTDPGVHATLKSLSSQIETMRSPDGSKKHPARTCDDLKLCHSAKQSGEYWIDPNQGSVEDAIKVYCNMETGETCISANPSSVPRKTWWASKSPDNKPVWYGLDMNRGSQFAYGDHQSPNTAITQMTFLRLLSKEASQNITYICKNSVGYMDDQAKNLKKAVVLKGANDLDIKAEGNIRFRYIVLQDTCSKRNGNVGKTVFEYRTQNVARLPIIDLAPVDVGGTDQEFGVEIGPVCFV

SEQ ID NO: 52 - DNA, 6192 bp, Homo sapiens
  CDS (87)..(5324)
gcgagtgactgcaccgagcccgagaagtcgccgcgccccgcagccgccccgactggttccccgccttgcccgtgggccccgccgggatggggaaccgccgggacctgggccagccgcgggccggtctctgcctgctcctggccgcgctgcagcttctgccggggacgcaggccgatcctgtggatgtcctgaaggccctgggtgtgcagggaggccaggctggggtccccgaggggcctggcttctgtccccagaggactccagagggtgaccgggcattcagaattggccaggccagcacgctcggcatccccacgtgggaactctttccagaaggccactttcctgagaacttctccttgctgatcaccttgcggggacagccagccaatcagtctgtcctgctgtccatttatgatgaaaggggtgcccggcagttgggcctggcactggggccagcgctgggtctcctaggtgaccccttccgccccctcccccagcaggtcaacctcacagatggcaggtggcaccgtgtggccgtcagcatagatggtgagatggtgaccctggtagctgactgtgaagctcagccccctgttttgggccatggcccccgcttcatcagcatagctggactcactgtgctggggacccaggaccttggggaaaagactttcgagggagacattcaggagctgctgataagcccagatcctcaggctgccttccaggcttgtgagcggtacctccccgactgtgacaacctggcaccggcagccacagtggctccccagggtgaaccagaaacccctcgtcctcggcggaaggggaagggaaaagggaggaagaaagggcgaggtcgcaaggggaagggcaggaaaaagaacaaggaaatttggacctcaagtccacctcctgactccgcagagaaccagacctccactgacatccccaagacagagactccagctccaaatctgcctccgacccccacgcctttggtcgtcacctccactgtgactactggactcaatgccacgatcctagagaggagcttggaccctgacagtggaaccgagctggggaccctggagaccaaggcagccagggaggatgaagaaggagatgattccaccatgggccctgacttccgggcagcagaatatccatctcggactcagttccagatctttcctggtgctggagagaaaggagcaaaaggagagcccgcagtgattgaaaaggggcagcagtttgagggacctccaggagccccaggaccccaaggggtggttggcccctcaggccctcccggccccccaggattccctggcgaccctggtccaccgggccctgctggcctcccaggaatccccggcattgatgggatccgaggcccaccgggcactgtgatcatgatgccgttccagtttgcaggcggctcctttaaaggccccccagtctcattccagcaggcccaggctcaggcagttctgcagcagactcagctctctatgaaaggcccccctggtccagtggggctcactgggcgcccaggccctgtgggtctccccgggcatccaggtctgaaaggagaggagggagcagaagggccacagggtccccgaggcctgcagggacctcatggaccccctggccgagtgggcaagatgggccgccctggagcagatggagctcggggcctcccaggggacactggacctaagggtgatcgtggcttcgatggcctccctgggctgcctggtgagaagggccaaaggggtgactttggccatgtggggcaacccggtcccccaggagaggatggtgagaggggagcagagggacctccagggcccactggccaggctggggagccgggtccacgaggactgcttggccccagaggctctcctggccccacgggtcgcccgggtgtgactggaattgatggtgctcctggtgccaaaggcaatgtgggtcctccaggagaaccaggccctccgggacagcagggaaaccatgggtcccagggactccccggtccccagggactcattggcactcctggggagaagggtccccctggaaacccaggaattccaggcctcccaggatccgatggccctctgggtcacccaggacatgagggccccacgggagagaaaggggctcagggtccaccagggtcggcaggccctccgggctatcctggacctcggggagtgaagggcacttcaggcaaccggggcctccagggggagaaaggcgagaagggagaggacggcttcccaggcttcaagggcgatgtggggctcaaaggtgatcaggggaaacccggagctccaggtccccggggagaggatggtcctgaggggccgaaggggcaggcggggcaggctggcgaggaggggcccccaggctcagctggggagaagggcaagcttggggtgccaggcctcccaggttatccaggacgccctggacctaagggatctattggatttcccggtcccctgggacccataggagagaaagggaagtcgggaaagacagggcagccaggcctggaaggagagcggggaccaccaggttcccgtggagagagggggcaaccgggtgccacagggcaaccaggccccaagggcgatgtgggccaggatggagcccctgggatccctggagaaaagggcctccctggtctgcaaggccctccaggattccctgggccaaagggcccccctggtcaccaaggtaaagatgggcgaccagggcaccctggacagagaggagaactgggcttccaaggtcagacaggcccgcctggaccagctggtgtcttaggccctcagggaaagacaggagaagtgggacctctaggtgaaagggggcctccaggcccccctggacctcctggtgaacaaggtcttcctggcctggaaggcagagagggggccaagggggaactgggaccaccaggaccccttgggaaagaagggccagctggactcaggggctttcccggccccaaagggggccctggggacccgggacctactggcttaaagggtgataagggccccccagggcccgtgggggccaatggctcccctggtgagcgcggtcctttgggcccagcaggaggcattggacttcctggccaaagtggcagcgaaggccccgttggccctgcaggcaagaaggggtcccggggagaacgtggcccccctggccccactggcaaagatgggatcccagggcccctggggcctctgggaccccctggagctgctgggccttctggcgaggaaggggacaagggggatgtgggtgcccccggacacaaggggagtaaaggcgataaaggagacgcgggcccacctggacaaccagggatacggggtcctgcaggacacccaggtcccccgggagcagacggggctcaggggcgccggggacccccaggcctctttgggcagaaaggagatgacggagtcagaggctttgtgggggtgattggccctcctggactgcaggggctgccaggccctccgggagagaaaggggaggtcggagacgtcgggtccatgggtccccatggagctccaggtcctcggggtccccaaggccccactggatcagagggcactccagggctgcctggaggagttggtcagccaggcgccgtgggtgagaagggtgagcgaggggacgctggagacccagggcctccaggagccccaggcatcccggggcccaagggagacattggtgaaaagggggactcaggcccatctggagctgctggacccccaggcaagaaaggtccccctggagaggatggagccaaagggagcgtgggccccacggggctgcccggagatctagggcccccaggagaccctggagtttcaggcatagatggttccccaggggagaagggagaccctggtgatgttgggggaccgggtccgcctggagcttctggggagcccggcgcccccgggccccccggcaagaggggtccttcaggccacatgggtcgagaaggcagagaaggggagaaaggtgccaagggggagccaggtcctgatgggcccccagggaggacgggtccaatgggggctagagggccccctggacgtgtggggcctgagggtcttcgagggatccctggccctgtgggtgaaccaggcctcctgggagcccctggacagatgggccctcctggccccctggggccctctggcctcccagggctgaagggagacactggccccaagggggaaaagggccacattggattgatcggtctcattggccccccgggagaagctggtgagaaaggagatcaggggttgccaggcgtgcagggaccccctggtcccaagggagaccctggtccccctggtcccattggctctctgggccaccctgggcccccaggtgtggcgggccctctaggacagaaaggctcaaaagggtctccggggtccatgggcccccgtggagacactggacctgcaggcccaccaggccccccgggtgcccctgccgagctgcatgggctgcgcaggcgccggcgcttcgtcccagtcccgcttccagtcgtggagggcggcctggaggaggtgctggcctcgctcacatcgctgagcttggagctggagcagctgcggcgtcctcccggcactgcggagcgcccgggcctcgtgtgccacgagctgcaccgcaaccacccgcacctgcctgatggggaatactggattgaccccaaccagggctgcgcgcgggactcgttcagggttttttgcaacttcacggcgggaggagagacctgcctctatcccgacaagaagtttgagatcgtgaaattggcctcctggtccaaggaaaagcctggaggctggtatagcacattccgtcgagggaagaagttctcctacgtggacgccgacgggtccccagtgaatgtcgtgcagctgaacttcctgaaactgctgagtgccacagctcgccagaacttcacctactcctgccagaatgcagctgcctggctggacgaagccacgggtgactacagccactccgcccgcttccttggcaccaatggagaggagctgtctttcaaccagacgacagcagccactgtcagcgtcccccaggatggctgccggctccggaaaggacagacgaagacccttttcgaattcagctcttctcgagcgggatttctgcccctgtgggatgtggcggccactgactttggccagacgaaccaaaagtttgggtttgaactgggccccgtctgcttcagcagctgagagtgtccggggtgggagggaccatgagggagccccagaatggggtgcatttggtgctgaggctttgaagccaccgtatttttcgttacctgtgactatggagccaatgggatgtgacttcgctcatcacggacagtcattccttctcctttccagggtgctgggggctggggttccctggcccaagggtccagcctcctctcaccccattccaggtggcatactgcagtctggctctttctcccctccctccccacccaagcctcacctccccaccccttgaacccccatgcaatgagcttctaactcagagctgatgaacaaaagcccccccacccccaatgcctgcctcctcactcctccgtcgctgcccttcacaccttttggtgctacccctccccagagttaagcactggatgtctcctgatcccaggctgggacccctacccccaccccctttgatcctttctacttccacggtgaaaggactgaggtcggactacagagggaagagggacttcccttgactgggttgtgtttcttttcctgcctcagcccagctctgcaaatcccctccccctgccccccacctccccaggctcaccttgccatgccaggtggtttggggaccaagatgttgggggggtgaatcaggatcctaatggtgctgccctatttatacctgggtctgtattaaaagggaaagtcccccctgttgtagatttcatctgcttcctccttagggaaggctgggatatgatgagagattccagcccaagcctggccccccaccgccaggccatagggcataatttgcatctcaaatctgagaataaactgatgaactgtgaaaaaaaaaaaaaaaaaa

SEQ ID NO: 53 - protein, 1745 aa, Homo sapiens
MGNRRDLGQPRAGLCLLLAALQLLPGTQADPVDVLKALGVQGGQAGVPEGPGFCPQRTPEGDRAFRIGQASTLGIPTWELFPEGHFPENFSLLITLRGQPANQSVLLSIYDERGARQLGLALGPALGLLGDPFRPLPQQVNLTDGRWHRVAVSIDGEMVTLVADCEAQPPVLGHGPRFISIAGLTVLGTQDLGEKTFEGDIQELLISPDPQAAFQACERYLPDCDNLAPAATVAPQGEPETPRPRRKGKGKGRKKGRGRKGKGRKKNKEIWTSSPPPDSAENQTSTDIPKTETPAPNLPPTPTPLVVTSTVTTGLNATILERSLDPDSGTELGTLETKAAREDEEGDDSTMGPDFRAAEYPSRTQFQIFPGAGEKGAKGEPAVIEKGQQFEGPPGAPGPQGVVGPSGPPGPPGFPGDPGPPGPAGLPGIPGIDGIRGPPGTVIMMPFQFAGGSFKGPPVSFQQAQAQAVLQQTQLSMKGPPGPVGLTGRPGPVGLPGHPGLKGEEGAEGPQGPRGLQGPHGPPGRVGKMGRPGADGARGLPGDTGPKGDRGFDGLPGLPGEKGQRGDFGHVGQPGPPGEDGERGAEGPPGPTGQAGEPGPRGLLGPRGSPGPTGRPGVTGIDGAPGAKGNVGPPGEPGPPGQQGNHGSQGLPGPQGLIGTPGEKGPPGNPGIPGLPGSDGPLGHPGHEGPTGEKGAQGPPGSAGPPGYPGPRGVKGTSGNRGLQGEKGEKGEDGFPGFKGDVGLKGDQGKPGAPGPRGEDGPEGPKGQAGQAGEEGPPGSAGEKGKLGVPGLPGYPGRPGPKGSIGFPGPLGPIGEKGKSGKTGQPGLEGERGPPGSRGERGQPGATGQPGPKGDVGQDGAPGIPGEKGLPGLQGPPGFPGPKGPPGHQGKDGRPGHPGQRGELGFQGQTGPPGPAGVLGPQGKTGEVGPLGERGPPGPPGPPGEQGLPGLEGREGAKGELGPPGPLGKEGPAGLRGFPGPKGGPGDPGPTGLKGDKGPPGPVGANGSPGERGPLGPAGGIGLPGQSGSEGPVGPAGKKGSRGERGPPGPTGKDGIPGPLGPLGPPGAAGPSGEEGDKGDVGAPGHKGSKGDKGDAGPPGQPGIRGPAGHPGPPGADGAQGRRGPPGLFGQKGDDGVRGFVGVIGPPGLQGLPGPPGEKGEVGDVGSMGPHGAPGPRGPQGPTGSEGTPGLPGGVGQPGAVGEKGERGDAGDPGPPGAPGIPGPKGDIGEKGDSGPSGAAGPPGKKGPPGEDGAKGSVGPTGLPGDLGPPGDPGVSGIDGSPGEKGDPGDVGGPGPPGASGEPGAPGPPGKRGPSGHMGREGREGEKGAKGEPGPDGPPGRTGPMGARGPPGRVGPEGLRGIPGPVGEPGLLGAPGQMGPPGPLGPSGLPGLKGDTGPKGEKGHIGLIGLIGPPGEAGEKGDQGLPGVQGPPGPKGDPGPPGPIGSLGHPGPPGVAGPLGQKGSKGSPGSMGPRGDTGPAGPPGPPGAPAELHGLRRRRRFVPVPLPVVEGGLEEVLASLTSLSLELEQLRRPPGTAERPGLVCHELHRNHPHLPDGEYWIDPNQGCARDSFRVFCNFTAGGETCLYPDKKFEIVKLASWSKEKPGGWYSTFRRGKKFSYVDADGSPVNVVQLNFLKLLSATARQNFTYSCQNAAAWLDEATGDYSHSARFLGTNGEELSFNQTTAATVSVPQDGCRLRKGQTKTLFEFSSSRAGFLPLWDVAATDFGQTNQKFGFELGPVCFSS

SEQ ID NO: 54 - DNA, 6960 bp, Homo sapiens
  CDS (319)..(5391)
acacagtactctcagcttgttggtggaagcccctcatctgccttcattctgaaggcagggcccggcagaggaaggatcagagggtcgcggccggagggtcccggccggtggggccaactcagagggagaggaaagggctagagacacgaagaacgcaaaccatcaaatttagaagaaaaagccctttgactttttccccctctccctccccaatggctgtgtagcaaacatccctggcgataccttggaaaggacgaagttggtctgcagtcgcaatttcgtgggttgagttcacagttgtgagtgcggggctcggagatggagccgtggtcctctaggtggaaaacgaaacggtggctctgggatttcaccgtaacaaccctcgcattgaccttcctcttccaagctagagaggtcagaggagctgctccagttgatgtactaaaagcactagattttcacaattctccagagggaatatcaaaaacaacgggattttgcacaaacagaaagaattctaaaggctcagatactgcttacagagtttcaaagcaagcacaactcagtgccccaacaaaacagttatttccaggtggaactttcccagaagacttttcaatactatttacagtaaaaccaaaaaaaggaattcagtctttccttttatctatatataatgagcatggtattcagcaaattggtgttgaggttgggagatcacctgtttttctgtttgaagaccacactggaaaacctgccccagaagactatcccctcttcagaactgttaacatcgctgacgggaagtggcatcgggtagcaatcagcgtggagaagaaaactgtgacaatgattgttgattgtaagaagaaaaccacgaaaccacttgatagaagtgagagagcaattgttgataccaatggaatcacggtttttggaacaaggattttggatgaagaagtttttgagggggacattcagcagtttttgatcacaggtgatcccaaggcagcatatgactactgtgagcattatagtccagactgtgactcttcagcacccaaggctgctcaagctcaggaacctcagatagatgagtatgcaccagaggatataatcgaatatgactatgagtatggggaagcagagtataaagaggctgaaagtgtaacagagggacccactgtaactgaggagacaatagcacagacggagataaatggccatggtgcatatggagagaaaggacagaaaggagaaccagcagtggttgagcctggtatgcttgtcgaaggaccaccaggaccagcaggacctgcaggtattatgggtcctccaggtctacaaggccccactggaccccctggtgaccctggcgataggggccccccaggacgtcctggcttaccaggggctgatggtctacctggtcctcctggtactatgttgatgttaccgttccgttatggtggtgatggttccaaaggaccaaccatctctgctcaggaagctcaggctcaagctattcttcagcaggctcggattgctctgagaggcccacctggcccaatgggtctaactggaagaccaggtcctgtgggggggcctggttcatctggggccaaaggtgagagtggtgatccaggtcctcagggccctcgaggcgtccagggtccccctggtccaacgggaaaacctggaaaaaggggtcgtccaggtgcagatggaggaagaggaatgccaggagaacctggggcaaagggagatcgagggtttgatggacttccgggtctgccaggtgacaaaggtcacaggggtgaacgaggtcctcaaggtcctccaggtcctcctggtgatgatggaatgaggggagaagatggagaaattggaccaagaggtcttccaggtgaagctggcccacgaggtttgctgggtccaaggggaactccaggagctccagggcagcctggtatggcaggtgtagatggccccccaggaccaaaagggaacatgggtccccaaggggagcctgggcctccaggtcaacaagggaatccaggacctcagggtcttcctggtccacaaggtccaattggtcctcctggtgaaaaaggaccacaaggaaaaccaggacttgctggacttcctggtgctgatgggcctcctggtcatcctgggaaagaaggccagtctggagaaaagggggctctgggtccccctggtccacaaggtcctattggatacccgggcccccggggagtaaagggagcagatggtgtcagaggtctcaagggatctaaaggtgaaaagggtgaagatggttttccaggattcaaaggtgacatgggtctaaaaggtgacagaggagaagttggtcaaattggcccaagaggggaagatggccctgaaggacccaaaggtcgagcaggcccaactggagacccaggtccttcaggtcaagcaggagaaaagggaaaacttggagttccaggattaccaggatatccaggaagacaaggtccaaagggttccactggattccctgggtttccaggtgccaatggagagaaaggtgcacggggagtagctggcaaaccaggccctcggggtcagcgtggtccaacgggtcctcgaggttcaagaggtgcaagaggtcccactgggaaacctgggccaaagggcacttcaggtggcgatggccctcctggccctccaggtgaaagaggtcctcaaggacctcagggtccagttggattccctggaccaaaaggccctcctggaccacctgggaaggatgggctgccaggacaccctgggcaacgtggggagactggatttcaaggcaagaccggccctcctgggccagggggagtggttggaccacagggaccaaccggtgagactggtccaataggggaacgtgggcatcctggccctcctggccctcctggtgagcaaggtcttcctggtgctgcaggaaaagaaggtgcaaagggtgatccaggtcctcaaggtatctcagggaaagatggaccagcaggattacgtggtttcccaggggaaagaggtcttcctggagctcagggtgcacctggactgaaaggaggggaaggtccccagggcccaccaggtccagttggctcaccaggagaacgtgggtcagcaggtacagctggcccaattggtttaccagggcgcccgggacctcagggtcctcctggtccagctggagagaaaggtgctcctggagaaaaaggtccccaagggcctgcagggagagatggagttcaaggtcctgttggtctcccagggccagctggtcctgccggctcccctggggaagacggagacaagggtgaaattggtgagccgggacaaaaaggcagcaagggtgacaagggagaaaatggccctcccggtcccccaggtcttcaaggaccagttggtgcccctggaattgctggaggtgatggtgaaccaggtcctagaggacagcaggggatgtttgggcaaaaaggtgatgagggtgccagaggcttccctggacctcctggtccaataggtcttcagggtctgccaggcccacctggtgaaaaaggtgaaaatggggatgttggtcccatggggccacctggtcctccaggcccaagaggccctcaaggtcccaatggagctgatggaccacaaggacccccagggtctgttggttcagttggtggtgttggagaaaagggtgaacctggagaagcagggaacccagggcctcctggggaagcaggtgtaggcggtcccaaaggagaaagaggagagaaaggggaagctggtccacctggagctgctggacctccaggtgccaaggggccaccaggtgatgatggccctaagggtaacccgggtcctgttggttttcctggagatcctggtcctcctggggaacctggccctgcaggtcaagatggtgttggtggtgacaagggtgaagatggagatcctggtcaaccgggtcctcctggcccatctggtgaggctggcccaccaggtcctcctggaaaacgaggtcctcctggagctgcaggtgcagagggaagacaaggtgaaaaaggtgctaagggggaagcaggtgcagaaggtcctcctggaaaaaccggcccagtcggtcctcagggacctgcaggaaagcctggtccagaaggtcttcggggcatccctggtcctgtgggagaacaaggtctccctggagctgcaggccaagatggaccacctggtcctatgggacctcctggcttacctggtctcaaaggtgaccctggctccaagggtgaaaagggacatcctggtttaattggcctgattggtcctccaggagaacaaggggaaaaaggtgaccgagggctccctggaactcaaggatctccaggagcaaaaggggatgggggaattcctggtcctgctggtcccttaggtccacctggtcctccaggtttaccaggtcctcaaggcccaaagggtaacaaaggctctactggacccgctggccagaaaggtgacagtggtcttccagggcctcctgggtctccaggtccacctggtgaagtcattcagcctttaccaatcttgtcctccaaaaaaacgagaagacatactgaaggcatgcaagcagatgcagatgataatattcttgattactcggatggaatggaagaaatatttggttccctcaattccctgaaacaagacattgagcatatgaaatttccaatgggtactcagaccaatccagcccgaacttgtaaagacctgcaactcagccatcctgacttcccagatggtgaatattggattgatcctaaccaaggttgctcaggagattccttcaaagtttactgtaatttcacatctggtggtgagacttgcatttatccagacaaaaaatctgagggagtaagaatttcatcatggccaaaggagaaaccaggaagttggtttagtgaatttaagaggggaaaactgctttcatacttagatgttgaaggaaattccatcaatatggtgcaaatgacattcctgaaacttctgactgcctctgctcggcaaaatttcacctaccactgtcatcagtcagcagcctggtatgatgtgtcatcaggaagttatgacaaagcacttcgcttcctgggatcaaatgatgaggagatgtcctatgacaataatccttttatcaaaacactgtatgatggttgtgcgtccagaaaaggctatgaaaagactgtcattgaaatcaatacaccaaaaattgatcaagtacctattgttgatgtcatgatcaatgactttggtgatcagaatcagaagttcggatttgaagttggtcctgtttgttttcttggctaagattaagacaaagaacatatcaaatcaacagaaaatataccttggtgccaccaacccattttgtgccacatgcaagttttgaataaggatggtatagaaaacaacgctgcatatacaggtaccatttaggaaataccgatgcctttgtgggggcagaatcacatggcaaaagctttgaaaatcataaagatataagttggtgtggctaagatggaaacagggctgattcttgattcccaattctcaactctccttttcctatttgaatttctttggtgctgtagaaaacaaaaaaagaaaaatatatattcataaaaaatatggtgctcattctcatccatccaggatgtactaaaacagtgtgtttaataaattgtaattattttgtgtacagttctatactgttatctgtgtccatttccaaaacttgcacgtgtccctgaattccatctgactctaattttatgagaattgcagaactctgatggcaataaatatatgtattatgaaaaaataaagttgtaatttctgatgactctaagtccctttctttggttaataataaaatgcctttgtatatattgatgttgaagagttcaattatttgatgtcgccaacaaaattctcagagggcaaaaatctggaagacttttggaagcacactctgatcaactcttctctgccgacagtcattttgctgaatttcagccaaaaatattatgcattttgatgctttattcaaggctatacctcaaactttttcttctcagaatccaggatttcacaggatacttgtatatatggaaaacaagcaagtttatatttttggacagggaaatgtgtgtaagaaagtatattaacaaatcaatgcctccgtcaagcaaacaatcatatgtatactttttttctacgttatctcatctccttgttttcagtgtgcttcaataatgcaggttaatattaaagatggaaattaagcaattatttatgaatttgtgcaatgttagattttcttatcaatcaagttcttgaatttgattctaagttgcatattataacagtctcgaaaattattttacttgcccaacaaatattacttttttcctttcaagataattttataaatcatttgacctacctaattgctaaatgaataacatatggtggactgttattaagagtatttgttttaagtcattcaggaaaatctaaacttttttttccactaaggtatttactttaaggtagcttgaaatagcaatacaatttaaaaattaaaaactgaattttgtatctattttaagtaatatatgtaagacttgaaaataaatgttttatttcttatataaagtgttaaattaattgataccagatttcactggaacagtttcaactgataatttatgacaaaagaacatacctgtaatattgaaattaaaaagtgaaatttgtcataaagaatttcttttatttttgaaatcgagtttgtaaatgtccttttaagaagggagatatgaatccaataaataaactcaagtcttggctacctggaaaaaaaaaaaaaaaaaa

SEQ ID NO: 55 - protein, 1690 aa, Homo sapiens
MEPWSSRWKTKRWLWDFTVTTLALTFLFQAREVRGAAPVDVLKALDFHNSPEGISKTTGFCTNRKNSKGSDTAYRVSKQAQLSAPTKQLFPGGTFPEDFSILFTVKPKKGIQSFLLSIYNEHGIQQIGVEVGRSPVFLFEDHTGKPAPEDYPLFRTVNIADGKWHRVAISVEKKTVTMIVDCKKKTTKPLDRSERAIVDTNGITVFGTRILDEEVFEGDIQQFLITGDPKAAYDYCEHYSPDCDSSAPKAAQAQEPQIDEYAPEDIIEYDYEYGEAEYKEAESVTEGPTVTEETIAQTEINGHGAYGEKGQKGEPAVVEPGMLVEGPPGPAGPAGIMGPPGLQGPTGPPGDPGDRGPPGRPGLPGADGLPGPPGTMLMLPFRYGGDGSKGPTISAQEAQAQAILQQARIALRGPPGPMGLTGRPGPVGGPGSSGAKGESGDPGPQGPRGVQGPPGPTGKPGKRGRPGADGGRGMPGEPGAKGDRGFDGLPGLPGDKGHRGERGPQGPPGPPGDDGMRGEDGEIGPRGLPGEAGPRGLLGPRGTPGAPGQPGMAGVDGPPGPKGNMGPQGEPGPPGQQGNPGPQGLPGPQGPIGPPGEKGPQGKPGLAGLPGADGPPGHPGKEGQSGEKGALGPPGPQGPIGYPGPRGVKGADGVRGLKGSKGEKGEDGFPGFKGDMGLKGDRGEVGQIGPRGEDGPEGPKGRAGPTGDPGPSGQAGEKGKLGVPGLPGYPGRQGPKGSTGFPGFPGANGEKGARGVAGKPGPRGQRGPTGPRGSRGARGPTGKPGPKGTSGGDGPPGPPGERGPQGPQGPVGFPGPKGPPGPPGKDGLPGHPGQRGETGFQGKTGPPGPGGVVGPQGPTGETGPIGERGHPGPPGPPGEQGLPGAAGKEGAKGDPGPQGISGKDGPAGLRGFPGERGLPGAQGAPGLKGGEGPQGPPGPVGSPGERGSAGTAGPIGLPGRPGPQGPPGPAGEKGAPGEKGPQGPAGRDGVQGPVGLPGPAGPAGSPGEDGDKGEIGEPGQKGSKGDKGENGPPGPPGLQGPVGAPGIAGGDGEPGPRGQQGMFGQKGDEGARGFPGPPGPIGLQGLPGPPGEKGENGDVGPMGPPGPPGPRGPQGPNGADGPQGPPGSVGSVGGVGEKGEPGEAGNPGPPGEAGVGGPKGERGEKGEAGPPGAAGPPGAKGPPGDDGPKGNPGPVGFPGDPGPPGEPGPAGQDGVGGDKGEDGDPGQPGPPGPSGEAGPPGPPGKRGPPGAAGAEGRQGEKGAKGEAGAEGPPGKTGPVGPQGPAGKPGPEGLRGIPGPVGEQGLPGAAGQDGPPGPMGPPGLPGLKGDPGSKGEKGHPGLIGLIGPPGEQGEKGDRGLPGTQGSPGAKGDGGIPGPAGPLGPPGPPGLPGPQGPKGNKGSTGPAGQKGDSGLPGPPGSPGPPGEVIQPLPILSSKKTRRHTEGMQADADDNILDYSDGMEEIFGSLNSLKQDIEHMKFPMGTQTNPARTCKDLQLSHPDFPDGEYWIDPNQGCSGDSFKVYCNFTSGGETCIYPDKKSEGVRISSWPKEKPGSWFSEFKRGKLLSYLDVEGNSINMVQMTFLKLLTASARQNFTYHCHQSAAWYDVSSGSYDKALRFLGSNDEEMSYDNNPFIKTLYDGCASRKGYEKTVIEINTPKIDQVPIVDVMINDFGDQNQKFGFEVGPVCFLG

SEQ ID NO: 56 - DNA, 1237 bp, Homo sapiens
  CDS (229)..(1101)
cacacagaagcggcagccaccgaggagggagcagtgccgggagccccgacggcgccttgctgcatggagctgggccgctgacagctgtcgctgcccgcagcctctgacctccctgggaccccggcgtctgaggctcatagtctgctccctgtcttctgtcagcctcagggcatccagcgtctcaggccgacctgggtccctgggacccggcgtttcggcttctcagccatggagcggtgcagccgctgccatcgcctcctcctcctcctacctctggtgctggggctgagcgcggccccaggctgggcaggtgcaccccctgtggatgtgctccgggccctgaggttcccctccctccctgatggtgtccggagagcgaaaggcatctgtccagctgatgtggcctaccgagtggcacgacctgcccagctcagtgcacccactcgccagcttttcccaggaggatttcccaaagatttctctctgctgactgttgtccggacccgccctggtctccaagctcccctcctgactctctacagtgcccagggtgtccgacagctgggcctggagctgggccgacctgtccgcttcctgtatgaagaccagactgggcggcctcaacctccctctcagccagtcttccgaggcctcagcctagcagatggcaagtggcaccgtgtggctgtggctgtgaagggccagtctgtcaccctcattgttgactgcaagaagcgagtcacccggcctctcccccgaagtgctcgtccagtattggacacccatggagtgatcatctttggtgcccgtattctggatgaagaagtctttgagggtgatgtccaggagctggccattgtcccaggggtccaggcagcctatgaatcatgtgaacagaaggagctggaatgcgaggggggccagagggaaagaccccaaaaccaacagcctcacagagcccagagatctccacagcagcaaccatcaagacttcacaggccacaaaatcaggaaccccagagccaggtgagggagctgggagaacccccaagtgcagcacaccccagagagggaagacacccaggcatctctcctccttagttgtcctccccaccctcatctccctattcccatcaccccctcctcctagtcctcattcatcagcctttttattttcatctaaaaataaaaagagttgttatgaagaaaaaaaaaaaaaaaaaaaaaaaaaaaaaaa

SEQ ID NO: 57 - protein, 290 aa, Homo sapiens
MERCSRCHRLLLLLPLVLGLSAAPGWAGAPPVDVLRALRFPSLPDGVRRAKGICPADVAYRVARPAQLSAPTRQLFPGGFPKDFSLLTVVRTRPGLQAPLLTLYSAQGVRQLGLELGRPVRFLYEDQTGRPQPPSQPVFRGLSLADGKWHRVAVAVKGQSVTLIVDCKKRVTRPLPRSARPVLDTHGVIIFGARILDEEVFEGDVQELAIVPGVQAAYESCEQKELECEGGQRERPQNQQPHRAQRSPQQQPSRLHRPQNQEPQSQVRELGEPPSAAHPREGRHPGISPP

SEQ ID NO: 58 - protein, 17 aa, Artificial Sequence
  Synthetic
GSPPGPPGPPGPPGPPG

SEQ ID NO: 59 - protein, 17 aa, Artificial Sequence
  Synthetic
GSPPGPPGPPGPPGPPG

SEQ ID NO: 60 - protein, 23 aa, Artificial Sequence
  Synthetic
GSGPPGPPGPPGPPGPPGPPGPP

SEQ ID NO: 61 - protein, 31 aa, Artificial Sequence
  Synthetic
GSGSGYIPEAPRDGQAYVRKDGEWVLLSTFL

SEQ ID NO: 62 - protein, 61 aa, Artificial Sequence
  Synthetic
GSGPPGPPGPPGPPGPPGPPGPPGPPGPPGPPGSGYIPEAPRDGQAYVRKDGEWVLLSTFL

SEQ ID NO: 63 - DNA, 213 bp, Artificial Sequence
  Synthetic
ggatcccctggtgccgcgtggcagcggtccgccgggcccgccgggcccgccgggtccgccgggacctccgggtcctcctggccctcctggtccgccgggacccccgggtccgccgggcagcggttatattccggaagcaccgcgtgatggtcaggcatacgtgcgtaaagatggcgaatgggttctgctgtctacctttctgtaagcggccgc

SEQ ID NO: 64 - DNA, 23 bp, Artificial Sequence
  Synthetic
gtgccgcgtggatccggtccgcc

SEQ ID NO: 65 - DNA, 31 bp, Artificial Sequence
  Synthetic
acgatgcggccgcttacagaaaggtagacag

SEQ ID NO: 66 - protein, 285 aa, Artificial Sequence
  Synthetic
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSGPPGPPGPPGPPGPPGPPGPPGPPGPPGPPGSGYIPEAPRDGQAYVRKDGEWVLLSTFL

SEQ ID NO: 67 - protein, 32 aa, Artificial Sequence
  Synthetic
GSGPPGPPGPPGPPGPPGPPGPPGPPGPPGPP

SEQ ID NO: 68 - DNA, 32 bp, Artificial Sequence
  Synthetic
gacccccgggtccgccgtgagcggttatattc

SEQ ID NO: 69 - DNA, 32 bp, Artificial Sequence
  Synthetic
gaatataaccgctcacggcggacccgggggtc

SEQ ID NO: 70 - DNA, 31 bp, Artificial Sequence
  Synthetic
ctggccctccttgacgggacccccgggtccg

SEQ ID NO: 71 - DNA, 31 bp, Artificial Sequence
  Synthetic
cggacccgggggtcccgtcaaggagggccag

SEQ ID NO: 72 - protein, 270 aa, Artificial Sequence
  Synthetic
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSGPPGPPGPPGPPGPPGSGYIPEAPRDGQAYVRKDGEWVLLSTFL

SEQ ID NO: 73 - protein, 276 aa, Artificial Sequence
  Synthetic
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSGPPGPPGPPGPPGPPGPPGPPGSGYIPEAPRDGQAYVRKDGEWVLLSTFL

SEQ ID NO: 74 - DNA, 27 bp, Artificial Sequence
  Synthetic
ggcagcggttatattccggaagcaccg

SEQ ID NO: 75 - DNA, 36 bp, Artificial Sequence
  Synthetic
atataaccgctgccggatccacgcggaaccagatcc

SEQ ID NO: 76 - DNA, 33 bp, Artificial Sequence
  Synthetic
cgcgtggatccggtcctcctggccctcctggtc

SEQ ID NO: 77 - DNA, 29 bp, Artificial Sequence
  Synthetic
ggatccacgcggaaccagatccgattttg

SEQ ID NO: 78 - DNA, 27 bp, Artificial Sequence
  Synthetic
ggcagcggttatattccggaagcaccg

SEQ ID NO: 79 - DNA, 33 bp, Artificial Sequence
  Synthetic
atataaccgctgccaggagggccaggaggaccc

SEQ ID NO: 80 - protein, 46 aa, Artificial Sequence
  Synthetic
GSGPPGPPGPPGPPGPPGSGYIPEAPRDGQAYVRKDGEWVLLSTFL

SEQ ID NO: 81 - protein, 52 aa, Artificial Sequence
  Synthetic
GSGPPGPPGPPGPPGPPGPPGPPGSGYIPEAPRDGQAYVRKDGEWVLLSTFL

(19) United States
(12) Patent Application Publication
(10) Pub. No.: US 2013/0116412 A1
(43) Pub. Date: May 9, 2013

(54) Production of Post-Translationally Hydroxylated Recombinant Proteins in Bacteria

(71) Applicants: Daniel M. Pinkas, Walnut Creek, CA (US); Sheng Ding, Stanford, CA (US); Annelise E. Barron, Palo Alto, CA (US)

(72) Inventors: Daniel M. Pinkas, Walnut Creek, CA (US); Sheng Ding, Stanford, CA (US); Annelise E. Barron, Palo Alto, CA (US)

(21) Appl. No.: 13/636,497

(22) PCT Filed: Apr. 1, 2011
(86) PCT No.: PCT/US11/30989

§ 371 Date: Jan. 28, 2013

Related U.S. Application Data
(60) Provisional application No. 61/376,591, filed on Aug. 24, 2010.
(60) Provisional application No. 61/320,621, filed on Apr. 2, 2010.

(51) Int. Cl. C12N 15/70

(52) U.S. Cl. CPC C12N 15/70
USPC 530/356; 435/252.3; 435/252.33; 435/68.1

(57) ABSTRACT

Bacterial cells capable of producing recombinant proteins, such as post-translationally hydroxylated recombinant proteins, methods and kits for producing recombinant proteins, such as post-translationally hydroxylated recombinant proteins, and particular post-translationally hydroxylated recombinant collagen molecules produced by the methods and cells disclosed herein are provided by this invention.

### CROSS-REFERENCE TO RELATED APPLICATIONS

This application is a U.S. National Phase of International Application No. PCT/US2011/030989, filed Apr. 1, 2011, which claims priority to U.S. Provisional patent application 61/376,591 filed Aug. 24, 2010, and U.S. Provisional patent application 61/320,621 filed Apr. 2, 2010 and which are incorporated herein by reference in their entirety.

The sequence listing submitted herewith is incorporated by reference in its entirety.

### BACKGROUND OF THE INVENTION

1. Field of the Invention

The present invention relates generally to the fields of cell biology, microbiology, and recombinant protein production, and particularly to bacterial cells capable of producing post-translationally hydroxylated recombinant proteins.

2. Description of Related Art

Collagen is an important structural protein in animals that constitutes about 30 percent by weight of all protein in the body, and is found in the skin, tendons, ligature, vasculature, musculature, organs, teeth, bones, and other tissues. Due to its physiological ubiquity, collagen is valuable for use in a variety of pharmaceutical, medicinal, surgical, cosmetic, and food-related applications, among others.

There are more than twenty-eight known types of naturally occurring collagenous proteins, and an even larger number of collagen monomers, each encoded by a separate gene, that make up the different collagen types in humans. The most common collagen types are I, II, III, and IV. Type-I collagen is the most abundant collagen type and is found in the skin, tendons, vasculature, ligature, organs, teeth, and bone; type-II collagen is found mainly in cartilage and in the vitreous humor of the eye; type-III collagen is a major component of granulation tissue and reticular fibers, is commonly found alongside type-I collagen, and is also found in artery walls, skin, intestines, and the uterus; type-IV collagen is found in basal lamina, in the lens of the eye, and in capillaries and nephron glomeruli. Type VI collagen is expressed by neuronal cells in the brain and has been found to be important in the injury response of neurons to the cytotoxicity of the Alzheimer's peptide, Aβ₁₋₄₂, so, for instance, might have therapeutic applications (Cheng et al., 2009, “Collagen VI protects neurons against Aβ toxicity,” Nature Neurosci. 12: 119-121). Collagenous domains are also found in both surfactant protein A and surfactant protein D, which have important immune and anti-inflammatory activities within the skin and on all mucosal surfaces in the human body, as well as in the recently discovered, blood-soluble adiponectin protein, which forms a variety of different multimers and is an important regulator of blood glucose in humans.

Structurally, fibrillar collagen protein polymers that are part of the extracellular matrix in the human body can be as long as 300 nm long and 1.5 nm in diameter and are trimers of polypeptides known as α chains, each of which folds into a left-handed polyproline helix. Together, the three α chains twist together to form a highly stable, right-handed coiled coil, also known as a triple helix. With type-I collagen (and possibly with all fibrillar collagens), each triple helix associates into a right-handed super-coil that is sometimes referred to as a collagen microfibril.

The amino acid residues within collagen alpha chains follow a regular sequence pattern, which is often Gly-Pro-Y or Gly-X-Hyp, where Hyp is (2S,4R)-4-hydroxyproline (an amino acid that is formed in vivo only by a post-translational modification of proline), and X and Y can be any amino acid residue. Gly is required at every third position because the assembly of the triple helix puts this residue at the interior (axis) of the helix, where there is no space for a side group larger than glycine's single hydrogen atom. Consequently, the rings of the Pro and Hyp residues point outward once the chain is folded into the triple helix conformation. Proper folding of the coiled-coil collagen structure is dependent in large part on Hyp residues on the surface of the α-chain helices, since the hydroxyproline hydroxyl (—OH) groups participate in hydrogen bonds that stabilize the triple helical structure, and also contribute to a key stereoelectronic effects that stabilize the collagen helix. Most collagen types contain approximately 10-20% hydroxyproline by weight. Thus, proper recombinant expression of collagen, such that stable and biomimetic folds and structures can be formed spontaneously, requires post-translational hydroxylation of a portion of proline residues to convert them to hydroxyproline residues.

Gelatin is a hydrolyzed form of collagen, generally monomeric collagen, which can be comprised of fragments of collagen rather than whole collagen. Gelatin has a large number of applications, particularly in food, photography, and cosmetics, where it is frequently used as a gelling agent, and in pharmaceuticals, where it is frequently used for coating tablets or for making capsules.

Expression of many exogenous genes is readily achievable in a variety of recombinant host-vector systems. Expression of biologically functional proteins, however, becomes difficult to obtain if the final formation of the protein requires extensive post-translational processing. Specifically, the difficulty in producing collagens using recombinant technology is due to the fact that many host cell types used for recombinant production, including in particular bacterial cells, do not possess the active hydroxylase enzyme necessary to post-translationally convert proline residues in collagen to hydroxyproline residues.

One such hydroxylase enzyme is prolyl-4-hydroxylase (P4H) that is involved in the synthesis of all collagens. The enzyme is required to hydroxylate prolyl residues to 4-hydroxyproline, for prolines that occur in the Y-position of the -Gly-X-Y- repeat sequences of collagen. Prockop et al., 1984, N. Engl. J. Med. 311: 376-386. Unless an appropriate number (or fraction) of Y-position prolyl residues are hydroxylated to 4-hydroxyproline by P4H, the newly synthesized chains do not properly and stably assemble and fold into the natural triple-helical conformation at 37° C. Moreover, if hydroxylation does not occur, the polypeptides remain non-helical, are poorly secreted by cells, and cannot self-assemble into collagen fibrils.

U.S. Pat. No. 5,928,922 disclosed the expression of active human prolyl-4-hydroxylase in insect cells.

US Patent Application Publication No. 2005/0164345 discloses recombinant production of human collagen in yeast, specifically Pichia spp., and insect cells.

Bacteria are used to produce many recombinant proteins, for the reasons of, inter alia, their robustness, ease, rapidity, and low cost of growth in unsupplemented or minimally supplemented media, and capacity for survival during high-density growth, which can yield large amounts of recombinant protein with cultures of relatively small volume. However, the expression of properly formed collagen triple helices in a bacterial recombinant system has not been reported. Unlike eukaryotic cells such as yeast and insect cells, bacteria are unable to produce active P4H, which requires an ascorbate co-factor that bacteria do not produce. Thus, there is a need in the art to provide a bacterial cell capable of producing properly formed collagen structures, as well as methods for successful recombinant expression of any post-translationally hydroxylated protein in bacteria; there are many possible target proteins that could be expressed in E. coli more cheaply than they are currently expressed in insect cells or yeast cells. Moreover, it is very often the case that recombinant proteins have a medical purpose. Proteins that are expressed in eukaryotic cells such as yeast cells, insect cells, or Chinese hamster ovary (CHO) cells will often be glycosylated by these cells, which can be either an advantage or a disadvantage. Glycosylation can be an advantage, when this post-translational modification is necessary for the biological activity of a protein; but it can also be a disadvantage, since the particular forms of glycosylation that are put onto recombinantly expressed proteins in these non-human eukaryotic cells can cause an immune response if they are used in humans for medicinal, surgical, or cosmetic purposes. Proteins that are expressed in bacteria are typically completely free of glycosylation, since this type of post-translational modification does not normally occur in bacteria. Hence, collagen proteins that are expressed in bacteria such as E. coli, if pure and properly folded, could be expected to be completely non-immunogenic. This could be important for many uses of these recombinantly expressed collagenous proteins.

### SUMMARY OF THE INVENTION

It is against the above background that the present invention provides certain advantages and advancements over the prior art.

Although this invention is not limited to specific advantages or functionality, it is noted that the invention provides bacterial cells capable of producing recombinant proteins comprising:
a. one or more nucleic acids encoding a sugar-1,4-lactone oxidase or a sugar-1,4-lactone dehydrogenase; and
b. one or more nucleic acids encoding an ascorbate-dependent biosynthetic enzyme.
   In certain embodiments, the sugar-1,4-lactone oxidase is D-arabinono-1,4-lactone oxidase. In certain other embodiments, the ascorbate-dependent biosynthetic is a hydroxylase, and in particular embodiments, the hydroxylase is prolyl-4-hydroxylase.

In various aspects of the invention the one or more nucleic acids encoding the sugar-1,4-lactone oxidase or sugar-1,4-lactone dehydrogenase comprise a first expression vector, and the one or more nucleic acids encoding the ascorbate-dependent biosynthetic enzyme comprise a second expression vector.

In further aspects of the invention, the nucleic acids encoding the sugar-1,4-lactone oxidase or sugar-1,4-lactone dehydrogenase and the ascorbate-dependent biosynthetic enzyme comprise a single expression vector.

In another aspect, the invention provides methods of making a post-translationally hydroxylated recombinant protein comprising expressing in a bacterial cell as disclosed herein one or more nucleic acids encoding a peptide or protein to be hydroxylated. In certain other embodiments, the ascorbate-dependent biosynthetic is a hydroxylase, and in particular embodiments, the hydroxylase is prolyl-4-hydroxylase.

In another aspect, the invention provides post-translationally hydroxylated recombinant collagen molecules produced by a method comprising the step of co-expressing in a bacterial cell as disclosed herein one or more nucleic acids encoding collagen, one or more nucleic acids encoding a sugar-1,4-lactone oxidase or a sugar-1,4-lactone dehydrogenase, and one or more nucleic acids encoding an ascorbate-dependent biosynthetic enzyme, wherein the ascorbate-dependent biosynthetic enzyme is a hydroxylase, particularly prolyl-4-hydroxylase.

In yet another aspect, the invention provides Gram-negative bacterial cells as disclosed herein capable of expressing recombinant proteins comprising one or more nucleic acids encoding an ascorbate-dependent biosynthetic enzyme or an ascorbate-analog-dependent biosynthetic enzyme, wherein the enzyme is expressed in the periplasmic space of the bacterial cell, and wherein ascorbate or an ascorbate analog is supplied exogeneously. In certain other embodiments, the ascorbate-dependent biosynthetic enzyme is a hydroxylase, and in particular embodiments, the hydroxylase is prolyl-4-hydroxylase.

In a further aspect, the invention provides kits for producing post-translationally hydroxylated recombinant proteins comprising bacterial cells as disclosed herein, and, optionally, instructions for use.

In yet another aspect, the invention provides methods of making a post-translationally hydroxylated recombinant protein comprising a) providing nucleic acids encoding said protein and one or more nucleic acids encoding an ascorbate-dependent biosynthetic enzyme or ascorbate-analog-dependent biosynthetic enzyme, b) co-expressing in the periplasmic space of a Gram-negative bacterial cell said protein and an ascorbate-dependent biosynthetic enzyme or ascorbate-analog-dependent biosynthetic enzyme, and c) providing ascorbate or an ascorbate analog exogeneously to the cell. In certain other embodiments, the ascorbate-dependent biosynthetic enzyme is a hydroxylase, and in particular embodiments, the hydroxylase is prolyl-4-hydroxylase.

In another aspect, the invention provides for an engineered bacterial cell-based system that is capable of producing post-translationally hydroxylated recombinant proteins comprising:
a. one or more nucleic acids encoding a sugar-1,4-lactone oxidase or a sugar-1,4-lactone dehydrogenase; and
b. one or more nucleic acids, present either as plasmids or potentially, as genes inserted directly into the bacterial genome, which encode an ascorbate-dependent biosynthetic enzyme.

In certain embodiments of any of the disclosed aspects, one or more of the nucleic acids encoding the sugar-1,4-lactone oxidase, the ascorbate-dependent biosynthetic enzyme, and the peptide or protein to be hydroxylated are incorporated into the bacterial chromosome.

In some embodiments, the hydroxylated recombinant proteins of the disclosed methods and products comprise a collagenous domain that is sufficiently hydroxylated to form a triple-helical structure.

In other embodiments, the disclosed methods and products comprise a hydroxylated recombinant protein comprising a foldon domain of SEQ ID NO: 61. In certain embodiments, the foldon domain is fused to a terminus of the hydroxylated recombinant protein and facilitates self-assembly of the protein into a triple-helical structure.

In certain embodiments, the bacterial cells of the products and methods of the invention are Escherichia coli, Bacillus spp., or Pseudomonas aeruginosa cells.

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings.

FIG. 1 shows a matrix-assisted laser desorption/ionization (MALDI) mass spectrum of gluththione-5-transferase-(proline-proline-glycine)₅ (GST-(PPG)₅), expressed in E. coli Origami2 cells without P4H.

FIG. 2 shows a MALDI mass spectrum of PPG₅ without P4H co-incubation, expressed in Origami 2 (DE3) competent cells and purified on glutathione agarose resin. Peak 1: glycine-serine-(PPG)₅+H⁺ (GS(PPG)₅+H⁺); Peak 2: GS(PPG)₅+Na⁺; Peak 3: GS(PPG)₅+Na⁺+−Na.

FIG. 3 shows a MALDI mass spectrum of PPG₅ with P4H incubation, expressed in Origami 2 (DE3) competent cells and purified on glutathione agarose resin. Peak 1: GS(PPG)₅+H⁺; Peak 2: GS(PPG)₅+OH+H⁺; Peak 3: GS(PPG)₅+Na⁺; Peak 4: GS(PPG)₅+OH+Na⁺; Peak 5: GS(PPG)₅+2OH+Na⁺; Peak 6: GS(PPG)₅+OH+Na⁺+−Na; Peak 7: GS(PPG)₅+3OH+Na⁺; Peak 8: GS(PPG)₅+2OH+Na⁺+−Na; Peak 9: GS(PPG)₅+3OH+Na⁺+−Na.

FIG. 4 shows liquid chromatography (LC) chromatograms from liquid chromatography-mass spectrometry (LC-MS) analyses. Top: GS(PPG)₅; bottom: GS(PPG)₅ incubated with P4H.

FIG. 5 shows LC-MS mass spectra of peaks in FIG. 4 with retention time around 8 min. Top: GS(PPG)₅ incubated with P4H; bottom: GS(PPG)₅ alone.

FIG. 6 shows a selected mass vs. retention time chromatogram of (PPG)₅ incubated with P4H. From bottom to top, (PPG)₅, (PPG)₅+1OH, (PPG)₅+2OH, (PPG)₅+3OH, (PPG)₅+4OH. Peptides comprising a greater number of hydroxylated residues had shorter retention times.

FIG. 7 shows growth of bacterial expression strains (BLR and Origami2-Ori2, Novagen) in M9 minimal media using different carbon sources. Lactone=L-gulono-1,4-lactone.

FIG. 8 shows growth of Origami2 cells in M9 minimal media supplemented with 10 mL of 100× vitamin stock solution (0.42 g/L riboflavin, 5.4 g/L pantothenic acid, 6 g/L niacin, 1.4 g/L pyridoxine, 0.06 g/L biotin, and 0.04 g/L folic acid) per 1000 mL, 10 mL of 100× trace metal stock solution (27 g/L FeCl₃.6H₂O, 2 g/L ZnCl₂.4H₂O, 2 g/L CaCl₂.6H₂O, 2 g/L Na₂MoO₄.2H₂O, 1.9 g/L CuSO₄.5H₂O, 0.5 g/L H₃BO₃, and 100 mL/L concentrated HCl) per 1000 mL, and 0.4% casamino acids. Asc=ascorbic acid. Lact=L-gulono-1,4-lactone.

FIG. 9 shows sodium dodecyl sulfate polyacrylamide gel electrophoresis (SDS-PAGE) of GST-(PPG)₅ after purification using glutathione resin of cultures induced at 37° C. for 4 h. Left to right: expression with no supplement; expression with supplement of 50 μM Fe(II)SO₄ and 5 mM ascorbate; expression with supplement of 100 μM Fe(II)SO₄ and 10 mM ascorbate; protein ladder.

FIG. 10 shows SDS-PAGE of GST-(PPG)₅ after purification using glutathione resin of cultures induced at 25° C. for 16 h. Left to right: expression with no supplement; expression with supplement of 50 μM Fe(II)SO₄ and 5 mM ascorbate; expression with supplement of 100 μM Fe(II)SO₄ and 10 mM ascorbate; protein ladder.

FIG. 11 shows chromatograms of (PPG)₅ peptides cleaved from GST-(PPG)₅ expressed and supplemented under the indicated conditions. Mass spectrometry indicated that the (PPG)₅ peptide eluted at 8.5-8.6 min, and hydroxylated peptides eluted at retention times of 8.1 min or less. Species at 9.1 and 14.6 min are unidentified small molecules.

FIGS. 12A though 12G show ultraviolet (UV) absorbance chromatograms of the GS(PPG)₅ peptide resulting from different incubation conditions. (FIG. 12A) positive control (in vitro hydroxylation); (FIG. 12B) negative control [GST-(PPG)₅ expressed without P4H or D-arabinono-1,4-lactone oxidase (ALO1)]; in vivo hydroxylation of GST-(PPG)₅ in cultures incubated with Fe(II)SO₄ and (FIG. 12C) L-ascorbic acid, (FIG. 12D) D-Arabinono-1,4-lactone, (FIG. 12E) L-Galactono-1,4-lactone, (FIG. 12F) L-Gulono-1,4-lactone, or (FIG. 12G) nothing additional.

FIGS. 13A though 13F show mass spectra of peaks with retention time around 8 min for in vivo hydroxylation of GS(PPG)₅ incubated with Fe(II)SO₄ (no lactones or ascorbic acid were added). Retention times (min) for each spectrum are: (FIG. 13A) glutathione-S-transferase-(proline-4-hydroxyproline-glycine)₅ (GS(POG)₅), 6.557; (FIG. 13B) GS(POG)₄(PPG), 6.860; (FIG. 13C) GS(POG)₃(PPG)₂, 7.331, (FIG. 13D) GS(POG)₂(PPG)₃, 7.734, (FIG. 13E) GS(POG)(PPG)₄, 8.138, (FIG. 13F) GS(PPG)₅, 8.642.

FIG. 14 shows MALDI results of the GS(PPG)₅ peptide from cells incubated with Fe(II)SO₄, but neither lactone nor ascorbic acid. Peak 1: GS(PPG)₅+H⁺, peak 2: GS(PPG)₅+Na⁺, peak 3: GS(POG)₁(PPG)₄+H⁺, peak 4: GS(POG)₂(PPG)₃+H⁺, peak 5: GS(POG)₃(PPG)₂+H⁺, peak 6: GS(POG)₂(PPG)₃+Na⁺+−Na, peak 7: GS(POG)₄(PPG)+H⁺, peak 8: GS(POG)₃(PPG)₂+Na⁺+−Na, peak 9: GS(POG)₄(PPG)+Na⁺+−Na; O=4-hydroxyproline.

FIGS. 15A through 15D show UV absorbance chromatograms of GS(PPG)₅ peptides from cultures expressed (FIG. 15A) in terrific broth without ALO1 gene, (FIG. 15B) in terrific broth with ALO1 gene, (FIG. 15C) in LB media with ALO1 gene, and (FIG. 15D) in M9 minimal media plus vitamins, minerals, and 0.4% casamino acids with ALO1 gene.

FIG. 16 shows the reaction catalyzed by P4H. P4H catalyzes the formation of peptidyl (2S,4R)-4-hydroxyproline from peptidyl L-proline and molecular oxygen. In the process the catalytic Fe²⁺ ion is oxidized to Fe³⁺ which requires reduction by L-ascorbate for catalysis.

FIGS. 17A through 17H show LC-MS analysis of (Pro-Pro-Gly)₅ peptides cytosolically hydroxylated in E. coli under various conditions. UV absorbance chromatograms of (Pro-Pro-Gly)₅ peptides from cultures expressing (FIG. 17A) both P4H and ALO1 in: (“PA1”) Terrific Broth, (“PA2”) M9 minimal media plus 0.4% tryptone and 0.4% glycerol, and (“PA3”) M9 minimal media plus 0.4% tryptone. (FIG. 17B) Cultures not expressing ALO1: (“NEG”) expressing neither P4H nor ALO1 in Terrific Broth, (“P1”) expressing P4H only in Terrific Broth, (“P2”) expressing P4H only in M9 minimal media plus 0.4% tryptone and 0.4% glycerol, and (“P3”) expressing P4H only in M9 minimal media plus 0.4% tryptone. Arrows indicate number of hydroxylated prolines in the associated peaks as determined by quadrupole mass detection. Mass spectra of peaks with 0-5 hydroxyls are shown in FIGS. 17C-17H), respectively.

FIG. 18 shows a plasmid map of activator/reporter plasmid pSD.COLADuet-1.GST-(PPG)₅.ALO1 (pSD1001), which encodes both P4H activator and activity reporter genes. The activator gene ALO1 encodes the protein D-arabinono 1,4-lactone oxidase (ALO1) from S. cerevisiae. The P4H activity reporter encodes a fusion of the affinity tag glutathione-S-transferase (GST) to the high affinity P4H substrate (Pro-Pro-Gly)₅ ((PPG)₅) with an intervening thrombin protease cleavage site. The thrombin cleavage site coincides with one of the BamHI endonuclease sites shown in the vector map.

FIG. 19 shows the relationship between hydroxylation level and the amount of tryptone in culture media. Hydroxylation levels are shown of (Pro-Pro-Gly)₅ peptides expressed in E. coli system. The culture media were M9 minimal media with different amounts of tryptone as a carbon source (0.4%, 0.8%, 1.2%, and 2.4%, respectively).

FIGS. 20A and 20B show the results of an in vitro P4H activity assay. UV absorbance chromatograms are shown of (Pro-Pro-Gly)₅ peptides from different treatments. 0.2 mg of purified unhydroxylated GST-(Pro-Pro-Gly)₅ was incubated in 50 mM Tris-HCl buffer, pH 7.8 containing bovine serum albumin (1 mg/mL), catalase (100 μg/mL), dithiothreitol (100 μM), FeSO₄ (50 μM), α-ketoglutarate (500 μM), and P4H (1.5 μM). FIG. 20A: 2 mM ascorbate or FIG. 20B: no ascorbate, was added to the final mixture. The reactions took place for 15 hours at 37° C. The samples were then incubated with thrombin. After boiling, the recovered peptides in the supernatant were analyzed by LC-MS.

FIGS. 21A and 21B show triple helix formation by P4H mediated hydroxylation of collagenous peptides in E. coli. FIG. 21A: The relationship between melting temperature of (Pro-Pro-Gly)₅-foldon and (Pro-Pro-Gly)₇-foldon and hydroxylation level. Squares represent (Pro-Pro-Gly)₅-foldon. Triangles represent (Pro-Pro-Gly)₇-foldon. FIG. 21B: Hydroxylation levels of (Pro-Pro-Gly)₅, (Pro-Pro-Gly)₅-foldon, (Pro-Pro-Gly), (Pro-Pro-Gly)₇-foldon, (Pro-Pro-Gly)₁₀, and (Pro-Pro-Gly)₁₀-foldon constructs co-expressed with both P4H and ALO in E. coli, using M9 minimal media plus 0.4% tryptone and 0.4% glycerol. Hydroxylation level is reported as the percentage of substrate prolines (proline in the Y position of X-Y-Glycine repeats) that were hydroxylated.

Skilled artisans will appreciate that elements in the figures are illustrated for simplicity and clarity and have not necessarily been drawn to scale. For example, the dimensions of some of the elements in the figures can be exaggerated relative to other elements to help improve understanding of the embodiment(s) of the present invention.

### DETAILED DESCRIPTION OF THE INVENTION

All publications, patents and patent applications cited herein are hereby expressly incorporated by reference for all purposes.

Methods well known to those skilled in the art can be used to construct expression vectors and recombinant bacterial cells according to this invention. These methods include in vitro recombinant DNA techniques, synthetic techniques, in vivo recombination techniques, and PCR techniques. See, for example, techniques as described in Maniatis et al., 1989, MOLECULAR CLONING: A LABORATORY MANUAL, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

Before describing the present invention in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or can not be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the terms “polynucleotide”, “nucleotide”, “oligonucleotide”, and “nucleic acid” can be used interchangeably to refer to nucleic acid comprising DNA, RNA, derivatives thereof, or combinations thereof.

In one aspect, the invention provides an engineered bacterial cell-based system that is capable of producing recombinant proteins, such as post-translationally hydroxylated recombinant proteins, comprising:
a. one or more nucleic acids encoding a sugar-1,4-lactone oxidase or a sugar-1,4-lactone dehydrogenase; and
b. one or more nucleic acids, present either as plasmids or potentially, as genes inserted directly into the bacterial genome, which encode an ascorbate-dependent biosynthetic enzyme.

In another aspect, the invention provides bacterial cells capable of expressing recombinant proteins, for example hydroxylated recombinant proteins, comprising nucleic acids encoding a sugar-1,4-lactone oxidase or a sugar-1,4-lactone dehydrogenase that is expressed thereby, and nucleic acids encoding an ascorbate-dependent biosynthetic enzyme that is expressed thereby. In certain embodiments, the ascorbate-dependent biosynthetic enzyme is a hydroxylase, particularly a prolyl-4-hydroxylase.

In various embodiments, the nucleic acids encoding the sugar-1,4-lactone oxidase or sugar-1,4-lactone dehydrogenase comprise a first expression vector and the nucleic acids encoding the ascorbate-dependent biosynthetic enzyme comprise a second expression vector, wherein each of the proteins encoded by each of the expression vectors is expressed in the cell comprising them.

In further embodiments, the nucleic acids encoding the sugar-1,4-lactone oxidase or sugar-1,4-lactone dehydrogenase and the ascorbate-dependent biosynthetic enzyme comprise a single expression vector, wherein each of the proteins encoded by the expression vector is expressed in the cell comprising it.

The bacterial cells that that can be used with the disclosed methods and products include any bacteria capable of producing a recombinant protein. Non-limiting examples of bacteria include Escherichia coli, Pseudomonas aeruginosa, Bacillus subtilis, and other Bacillus spp.

In various embodiments, the bacterial cells have a cytoplasmic environment with a relatively high reduction-oxidation (redox) potential, and are thus characterized by a relatively oxidizing cytoplasm, in order to facilitate disulfide bond formation in one or more of the recombinantly expressed proteins. The bacterial cells can have an oxidizing cytoplasm, inter alia, as a consequence of mutations in genes normally associated with maintaining a low redox potential in the cytoplasm, such as thioredoxin reductase (trxB) and glutathione reductase (gor).

In particular embodiments, the bacterial cells are capable of expressing catalase, an enzyme that functions to catalyze the decomposition of hydrogen peroxide to water and oxygen.

In other particular embodiments, catalase is a eukaryotic enzyme, i.e. an enzyme produced in a eukaryotic species including species from yeast, fungi, plants, and animals.

As used herein, the terms “hydroxylation” and “hydroxylated” refer to the chemical addition of a hydroxyl (—OH) group to an amino acid, most often to the side chain moiety of the amino acid. For example, when post-translationally hydroxylated, the amino acid proline becomes 4-hydroxy-L-proline, also known as (2S,4R)-4-hydroxyproline or hydroxyproline (Hyp). Other non-limiting examples of hydroxylated amino acids include 5-hydroxylysine, β-hydroxyaspartate (β-hydroxyaspartic acid), and β-hydroxyasparagine.

As used herein, the term “sugar” refers to any monosaccharide or disaccharide. In certain embodiments, the sugar is D-arabinose, L-gulose, D-glucose, or L-galactose; in certain preferred embodiments, the sugar is D-arabinose.

As used herein, the term “sugar-1,4-lactone oxidase” refers to any enzyme capable of catalyzing the chemical oxidation of a sugar-1,4-lactone, particularly those sugar-1,4-lactone oxidases that are involved in ascorbate biosynthesis, and capable of catalyzing the dehydrogenation of a sugar 1,4-lactone for the purpose of using the dehydrogenated sugar to activate the hydroxylase. For example, the enzyme D-arabinono-1,4-lactone oxidase (ALO1) catalyzes the conversion of D-arabinono-1,4-lactone and oxygen into D-erythro-ascorbate and hydrogen peroxide. In certain embodiments, the sugar-1,4-lactone oxidase is D-arabinono-1,4-lactone oxidase, L-gulono-1,4-lactone oxidase, or D-glucono-1,4-lactone oxidase.

In particular embodiments of all aspects provided by the invention, the sugar-1,4-lactone oxidase is a eukaryotic enzyme, i.e. an enzyme produced in a eukaryotic species including without limitation species from yeast, fungi, plants, and animals, or an enzyme such as bacterial D-arabinono-1,4-lactone oxidase, L-gulono-1,4-lactone oxidase, or D-glucono-1,4-lactone oxidase; and the sugar-1,4-lactone dehydrogenase is D-arabinose dehydrogenase, L-gulono-1,4-lactone dehydrogenase, L-gulono-γ-lactone dehydrogenase, D-glucose dehydrogenase, L-galactono-1,4-lactone dehydrogenase, L-galactono-γ-lactone dehydrogenase, L-sorbosone dehydrogenase, or 2-ketogluconate dehydrogenase.

As used herein, the terms “sugar-1,4-lactone dehydrogenase” and “sugar dehydrogenase” can be used interchangeably to refer to any enzyme capable of catalyzing the chemical dehydrogenation or oxidation of a sugar-1,4-lactone or a sugar, particularly those dehydrogenases involved in ascorbate biosynthesis.

Exemplary GenBank Accession Numbers for specific embodiments of such enzymes include: D-arabinono-1,4-lactone oxidase: U40390 (SEQ ID NO: 1, nucleotide; SEQ ID NO: 2, protein), from Saccharomyces cerevisiae; L-gulono-1,4-lactone oxidase (L-gulono-γ-lactone oxidase, L-gulono-1,4-lactone dehydrogenase, L-gulono-γ-lactone dehydrogenase): AY453064 (SEQ ID NO: 3, nucleotide; SEQ ID NO: 4, protein), from Mus musculus; L-galactono-1,4-lactone dehydrogenase (L-galactono-γ-lactone dehydrogenase): NM_{—}001125317 (SEQ ID NO: 5, nucleotide: SEQ ID NO: 6, protein), from Arabidopsis thaliana; and 2-ketogluconate dehydrogenase (2-ketogluconate reductase): XM_{—}001940605 (SEQ ID NO: 7, nucleotide; SEQ ID NO: 8, protein), from Pyrenophora tritici-repentis Pt-1C-BFP.

In particular embodiments, the sugar-1,4-lactone oxidase is D-arabinono-1,4-lactone oxidase (ALO1), particularly Saccharomyces cerevisiae D-arabinono-1,4-lactone oxidase: GenBank Accession No. U40390 (SEQ ID NO: 1, nucleotide; SEQ ID NO: 2, protein).

As used herein, the terms “ascorbate-dependent biosynthetic enzyme” and “ascorbate-analog-dependent biosynthetic enzyme” can be used interchangeably to refer to any biosynthetic enzyme that is active only in the presence of ascorbate, an ascorbate-analog, ascorbic acid, or an ascorbic acid analog co-factor. Non-limiting examples of ascorbate-dependent biosynthetic enzymes include dopamine β-hydroxylase, peptidylglycine α-amidating monooxygenase, 4-hydroxyphenylpyruvate dioxygenase, prolyl-4-hydroxylase, prolyl-3-hydroxylase, lysyl-5-hydroxylase, thymine 7-hydroxylase, pyrimidine deoxyribonucleoside 2′-hydroxylase, deoxyuridine (uridine) 1′-hydroxylase, ε-N-trimethyl-L-lysine hydroxylase, γ-butyrobetaine hydroxylase; such enzymes are discussed, for example, in Englard and Seifter (1986), “The biochemical functions of ascorbic acid.” Annual Review of Nutrition 6: 365-406, incorporated herein by reference in its entirety.

In certain embodiments of all aspects provided by the invention, the ascorbate-dependent biosynthetic enzyme is a hydroxylase, such as, for example, prolyl-4-hydroxylase (P4H), prolyl-3-hydroxylase, HIF prolyl hydroxylase, lysyl-5-hydroxylase, aspartyl beta-hydroxylase, asparaginyl beta-hydroxylase, or HIF asparaginyl hydroxylase from mammalian species including without limitation human, mouse, rat, pig, or cow.

Exemplary GenBank Accession Numbers are further provided herein: wild-type human prolyl-4-hydroxylase alpha subunit: NM_{—}000917 (SEQ ID NO: 9, nucleotide; SEQ ID NO: 10, protein); and wild-type human prolyl-4-hydroxylase beta subunit: NM_{—}000918 (SEQ ID NO: 11, nucleotide; SEQ ID NO: 12, protein); prolyl-3-hydroxylase, Homo sapiens: NM_{—}018192 (SEQ ID NO: 13, nucleotide; SEQ ID NO: 14, protein); HIF prolyl hydroxylase, Homo sapiens: NM_{—}022051 (SEQ ID NO: 15, nucleotide; SEQ ID NO: 16, protein); lysyl-5-hydroxylase (procollagen-lysine, 2-oxoglutarate 5-dioxygenase 3): NM_{—}001084 (SEQ ID NO: 17, nucleotide; SEQ ID NO: 18, protein) from Homo sapiens; aspartyl beta-hydroxylase or asparaginyl beta-hydroxylase: NM_{—}004318 (SEQ ID NO: 19, nucleotide; SEQ ID NO: 20, protein) from Homo sapiens; and HIF asparaginyl hydroxylase: NM_{—}017902 (SEQ ID NO: 21, nucleotide; SEQ ID NO: 22, protein) from Homo sapiens.

In particular embodiments, the ascorbate-dependent biosynthetic enzyme is prolyl-4-hydroxylase, preferably wild-type human prolyl-4-hydroxylase alpha subunit, GenBank Accession No. NM_{—}000917 (SEQ ID NO: 9, nucleotide; SEQ ID NO: 10, protein); and wild-type human prolyl-4-hydroxylase beta subunit, GenBank Accession No. NM_{—}000918 (SEQ ID NO: 11, nucleotide; SEQ ID NO: 12, protein).

In other particular embodiments, the ascorbate-dependent biosynthetic enzyme comprises prolyl-4-hydroxylase, preferably human prolyl-4-hydroxylase alpha subunit as described in Kersteen et al., 2004, Protein Purification and Expression 38: 279-291.

In further embodiments, the bacterial cells further comprise one or more nucleic acids encoding a peptide or protein to be hydroxylated that is expressed by the cells.

The fraction of residues that are post-translationally hydroxylated according to the products and methods of the invention may be modulated by altering the temperature at which the host cells are grown, typically from 13-37° C., with a higher fraction of hydroxylated residues occurring at higher temperatures. Alternatively, the expression constructs may be designed such that the promoter used in conjunction with the sugar-1,4-lactone oxidase (or dehydrogenase) is different from the promoter for the ascorbate-dependent biosynthetic enzyme, and can thus be induced differentially; for example, the nucleic acid encoding the sugar-1,4-lactone oxidase could be placed under transcriptional control of the lac operon (inducible with a molecule such as IPTG (isopropyl β-D-1-thiogalactopyranoside)), whereas the nucleic acid encoding the ascorbate-dependent biosynthetic enzyme could be placed under control of the TetR repressor (which could be separately induced by the presence or absence of a molecule such as tetracycline).

It is contemplated that more than one type of transcriptional expression system (such as the lac operon or TetR repressor) may be used advantageously in conjunction with the products and methods of the invention. Depending on the individual characteristics of the sugar-1,4-lactone dehydrogenase, the ascorbate-dependent biosynthetic enzyme, or the peptide or protein to be post-translationally modified, it may be advantageous to have temporal control over the expression of the various gene and protein products of the invention. For example, in the case that a protein, which is unstable in its unhydroxylated form, is to be post-translationally hydroxylated in a cell of the invention, it may be advantageous to first induce the expression of a hydroxylase and a sugar-1,4-lactone dehydrogenase (together under the control of a first transcriptional expression system, such as the lac operon), and later induce the expression of the protein to be hydroxylated (under the control of a second transcriptional expression system, such as the TetR repressor) in order to minimize the amount of time the protein is present in its unhydroxylated form.

Accordingly, in certain embodiments, the one or more nucleic acids encoding the ascorbate-dependent biosynthetic enzyme and the sugar-1,4-lactone oxidase or sugar-1,4-lactone dehydrogenase comprise a first expression vector, and the one or more nucleic acids encoding the peptide or protein to be hydroxylated comprise a second expression vector, wherein each of the proteins encoded by each of the expression vectors is expressed in the cell comprising them.

In other embodiments, the one or more nucleic acids encoding the sugar-1,4-lactone oxidase or sugar-1,4-lactone dehydrogenase comprise a first expression vector; the one or more nucleic acids encoding the ascorbate-dependent biosynthetic enzyme comprise a second expression vector; and the one or more nucleic acids encoding the peptide or protein to be hydroxylated comprise a third expression vector, wherein each of the proteins encoded by each of the expression vectors is expressed in the cell comprising them.

In further embodiments, the one or more nucleic acids encoding the sugar-1,4-lactone oxidase or sugar-1,4-lactone dehydrogenase and the peptide or protein to be hydroxylated comprise a first expression vector, and the one or more nucleic acids encoding the ascorbate-dependent biosynthetic enzyme comprise a second expression vector, wherein each of the proteins encoded by each of the expression vectors is expressed in the cell comprising them.

In yet other embodiments, the one or more nucleic acids encoding the ascorbate-dependent biosynthetic enzyme and the peptide or protein to be hydroxylated comprise a first expression vector, and the one or more nucleic acids encoding the sugar-1,4-lactone oxidase or sugar-1,4-lactone dehydrogenase comprise a second expression vector, wherein each of the proteins encoded by each of the expression vectors is expressed in the cell comprising them.

In yet other embodiments, the nucleic acids encoding the sugar-1,4-lactone oxidase or sugar-1,4-lactone dehydrogenase, the ascorbate-dependent biosynthetic enzyme, and the peptide or protein to be hydroxylated comprise a single expression vector, wherein each of the proteins encoded by the expression vector is expressed in the cell comprising it.

In various embodiments of this aspect, the sugar-1,4-lactone oxidase is D-arabinono-1,4-lactone oxidase, L-gulono-1,4-lactone oxidase, or D-glucono-1,4-lactone oxidase; and the sugar-1,4-lactone dehydrogenase is D-arabinose dehydrogenase, L-gulono-1,4-lactone dehydrogenase, L-gulono-γ-lactone dehydrogenase, D-glucose dehydrogenase, L-galactono-1,4-lactone dehydrogenase, L-galactono-γ-lactone dehydrogenase, L-sorbosone dehydrogenase, or 2-ketogluconate dehydrogenase. In particular embodiments the sugar-1,4-lactone oxidase is D-arabinono-1,4-lactone oxidase, preferably Saccharomyces cerevisiae D-arabinono-1,4-lactone oxidase.

In certain other embodiments, the ascorbate-dependent biosynthetic enzyme is prolyl-4-hydroxylase, prolyl-3-hydroxylase, HIF prolyl hydroxylase, lysyl-5-hydroxylase, aspartyl beta-hydroxylase, asparaginyl beta-hydroxylase, or HIF asparaginyl hydroxylase, and in particular embodiments, the ascorbate-dependent biosynthetic enzyme is prolyl-4-hydroxylase, preferably human prolyl-4-hydroxylase.

In other embodiments, the peptide or protein to be hydroxylated is collagen. As used herein, the term “collagen” refers to any member of a family of homotrimeric and heterotrimeric proteins found in the tissues of animals as discussed above.

In another aspect, the invention provides methods of making a post-translationally hydroxylated recombinant protein comprising expressing in a bacterial cell as disclosed herein one or more nucleic acids encoding a peptide or protein to be hydroxylated that is expressed thereby.

In certain embodiments of the methods of making post-translationally hydroxylated recombinant proteins disclosed herein, the bacterial cell comprises a first expression vector comprising the one or more nucleic acids encoding the sugar-1,4-lactone oxidase or sugar-1,4-lactone dehydrogenase; a second expression vector comprising the one or more nucleic acids encoding the ascorbate-dependent biosynthetic enzyme; and a third expression vector comprising the one or more nucleic acids encoding the peptide or protein to be hydroxylated, wherein each of the proteins encoded by each of the expression vectors is expressed in the cell comprising them.

In other embodiments of the methods of making post-translationally hydroxylated recombinant proteins disclosed herein, the bacterial cell comprises a first expression vector comprising the one or more nucleic acids encoding the sugar-1,4-lactone oxidase or sugar-1,4-lactone dehydrogenase and the peptide or protein to be hydroxylated; and a second expression vector comprising the one or more nucleic acids encoding the ascorbate-dependent biosynthetic enzyme, wherein each of the proteins encoded by each of the expression vectors is expressed in the cell comprising them.

In other embodiments of the methods of making post-translationally hydroxylated recombinant proteins disclosed herein, the bacterial cell comprises a first expression vector comprising the one or more nucleic acids encoding the ascorbate-dependent biosynthetic enzyme and the peptide or protein to be hydroxylated; and a second expression vector comprising the one or more nucleic acids encoding the sugar-1,4-lactone oxidase or sugar-1,4-lactone dehydrogenase, wherein each of the proteins encoded by each of the expression vectors is expressed in the cell comprising them.

In further embodiments of the methods of making post-translationally hydroxylated recombinant proteins disclosed herein, the bacterial cell comprises a first expression vector comprising the one or more nucleic acids encoding the ascorbate-dependent biosynthetic enzyme and the sugar-1,4-lactone oxidase or sugar-1,4-lactone dehydrogenase; and a second expression vector comprising the one or more nucleic acids encoding the peptide or protein to be hydroxylated, wherein each of the proteins encoded by each of the expression vectors is expressed in the cell comprising them.

In still further embodiments of the methods of making post-translationally hydroxylated recombinant proteins disclosed herein, the bacterial cell comprises an expression vector comprising the nucleic acids encoding the sugar-1,4-lactone oxidase or sugar-1,4-lactone dehydrogenase, the ascorbate-dependent biosynthetic enzyme, and the peptide or protein to be hydroxylated, wherein each of the proteins encoded by the expression vector is expressed in the cell comprising it.

In certain embodiments, the sugar-1,4-lactone oxidase is D-arabinono-1,4-lactone oxidase, L-gulono-1,4-lactone oxidase, or D-glucono-1,4-lactone oxidase; and the sugar-1,4-lactone dehydrogenase is D-arabinose dehydrogenase, L-gulono-1,4-lactone dehydrogenase, L-gulono-γ-lactone dehydrogenase, D-glucose dehydrogenase, L-galactono-1,4-lactone dehydrogenase, L-galactono-γ-lactone dehydrogenase, L-sorbosone dehydrogenase, or 2-ketogluconate dehydrogenase. In particular embodiments the sugar-1,4-lactone oxidase is D-arabinono-1,4-lactone oxidase, preferably Saccharomyces cerevisiae D-arabinono-1,4-lactone oxidase.

In further embodiments, the ascorbate-dependent biosynthetic enzyme is prolyl-4-hydroxylase, prolyl-3-hydroxylase, HIF prolyl hydroxylase, lysyl-5-hydroxylase, aspartyl beta-hydroxylase, asparaginyl beta-hydroxylase, or HIF asparaginyl hydroxylase, and in particular embodiments, the ascorbate-dependent biosynthetic enzyme is prolyl-4-hydroxylase, preferably human prolyl-4-hydroxylase.

In various aspects of the disclosed methods and products, the peptide or protein to be hydroxylated is collagen.

In further aspect, the invention provides post-translationally hydroxylated recombinant collagen molecules, produced in a bacterial cell comprising nucleic acids encoding collagen, nucleic acids encoding a sugar-1,4-lactone oxidase or sugar-1,4-lactone dehydrogenase, and nucleic acids encoding an ascorbate-dependent biosynthetic enzyme are co-expressed in a bacterial cell.

In particular embodiments, nucleic acids encoding the sugar-1,4-lactone oxidase or sugar-1,4-lactone dehydrogenase comprise a first expression vector; the nucleic acids encoding the ascorbate-dependent biosynthetic enzyme comprise a second expression vector; and the nucleic acids encoding collagen comprise a third expression vector, wherein each of the proteins encoded by each of the expression vectors is expressed in the cell comprising them.

In further embodiments, nucleic acids encoding the sugar-1,4-lactone oxidase or sugar-1,4-lactone dehydrogenase and collagen comprise a first expression vector, and nucleic acids encoding the ascorbate-dependent biosynthetic enzyme comprise a second expression vector, wherein each of the proteins encoded by each of the expression vectors is expressed in the cell comprising them.

In yet further embodiments, nucleic acids encoding the ascorbate-dependent biosynthetic enzyme and collagen comprise a first expression vector, and the nucleic acids encoding the sugar-1,4-lactone oxidase or sugar-1,4-lactone dehydrogenase comprise a second expression vector, wherein each of the proteins encoded by each of the expression vectors is expressed in the cell comprising them.

In still further embodiments, nucleic acids encoding the sugar-1,4-lactone oxidase or sugar-1,4-lactone dehydrogenase, the ascorbate-dependent biosynthetic enzyme, and collagen comprise a single expression vector, wherein each of the proteins encoded by the expression vector is expressed in the cell comprising it.

In various embodiments, the sugar-1,4-lactone oxidase is D-arabinono-1,4-lactone oxidase, L-gulono-1,4-lactone oxidase, or D-glucono-1,4-lactone oxidase; and the sugar-1,4-lactone dehydrogenase is D-arabinose dehydrogenase, L-gulono-1,4-lactone dehydrogenase, L-gulono-γ-lactone dehydrogenase, D-glucose dehydrogenase, L-galactono-1,4-lactone dehydrogenase, L-galactono-γ-lactone dehydrogenase, L-sorbosone dehydrogenase, or 2-ketogluconate dehydrogenase. In particular embodiments the sugar-1,4-lactone oxidase is D-arabinono-1,4-lactone oxidase, preferably Saccharomyces cerevisiae D-arabinono-1,4-lactone oxidase.

In yet other certain embodiments, the ascorbate-dependent biosynthetic enzyme is prolyl-4-hydroxylase, prolyl-3-hydroxylase, HIF prolyl hydroxylase, lysyl-5-hydroxylase, aspartyl beta-hydroxylase, asparaginyl beta-hydroxylase, or HIF asparaginyl hydroxylase, and in particular embodiments, the ascorbate-dependent biosynthetic enzyme is prolyl-4-hydroxylase, preferably human prolyl-4-hydroxylase.

DNA encoding any collagen monomer, such as α1(I) (GenBank Accession No. NM_{—}000088; SEQ ID NOS: 40 [nucleotide] and 41 [amino acid]), α2(I) (GenBank Accession No. NM_{—}000089; SEQ ID NOS: 42 [nucleotide] and 43 [amino acid]), α1(II) (GenBank Accession No. NM_{—}001844; SEQ ID NOS: 44 [nucleotide] and 45 [amino acid]), α1(III) (GenBank Accession No. NM_{—}000090; SEQ ID NOS: 46 [nucleotide] and 47 [amino acid]), α1(V) (GenBank Accession No. NM_{—}000093; SEQ ID NOS: 48 [nucleotide] and 49 [amino acid]), α2(V) (GenBank Accession No. NM_{—}000393; SEQ ID NOS: 50 [nucleotide] and 51 [amino acid]), α3(V) (GenBank Accession No. NM_{—}015719; SEQ ID NOS: 52 [nucleotide] and 53 [amino acid]), α1(XI) (GenBank Accession No. NM_{—}001168249; SEQ ID NOS: 54 [nucleotide] and 55 [amino acid]), and α2(XI) (GenBank Accession No. NM_{—}001163771; SEQ ID NOS: 56 [nucleotide] and 57 [amino acid]) collagen monomers, can be used advantageously in the products and methods of the disclosure. DNA can be obtained by any method from any source known in the art, such as isolation from cDNA or genomic libraries, amplification from an available template, or chemical synthesis. Using methods known in the art, de novo synthesis or modification of an existing DNA can also be used to produce DNA encoding variants.

For use in this invention, DNA encoding a collagen molecule or any other protein to be post-translationally hydroxylated is introduced inter alia by cloning into an expression vector. As is well known in the art, the particular details of the expression vector can vary according to the desired characteristics of the expression system, and to the type of host cell to be used. For example, promoters and promoter/operators operative in bacterial cells, such as the araB, trp, lac, gal, tac (a hybrid of the trp and lac promoter/operator), and T7, can be useful in accordance with the instant disclosure. The expression vector can also include a signal sequence that directs transport of the synthesized peptide into the periplasmic space; alternatively, expression can be directed intracellularly. In particular embodiments, said promoters and promoter/operators are inducible by inducer molecules including, inter alia, IPTG and tetracycline.

In particular embodiments, the expression vector can also comprise a marker that enables host cells containing the expression construct (a “selectable marker”) to be selected. Selectable markers are well known in the art. For example, the selectable marker can be a resistance gene, such as an antibiotic resistance gene (e.g., the neo^{r} gene which confers resistance to the antibiotic gentamycin), or it can be a gene which complements an auxotrophy of the host cell. The expression construct can also contain sequences which act as an “ARS” (autonomous replicating sequence) that permit the expression construct to replicate in the host cell without being integrated into the host cell chromosome. Origins of replication for bacterial plasmids are well known. So, for example, the expression construct can also comprise an ARS (“ori”) as well as a selectable marker useful for selection transformed cells.

In a further aspect, the invention provides Gram-negative bacterial cells capable of expressing recombinant proteins, for example hydroxylated recombinant proteins, comprising nucleic acids encoding an ascorbate-dependent biosynthetic enzyme or an ascorbate-analog-dependent biosynthetic enzyme, wherein the enzyme is expressed in the periplasmic space of the bacterial cell, and wherein exogenous ascorbate or an ascorbate analog is supplied to the cell. Since the bacterial periplasm is a relatively oxidizing environment, this aspect of the disclosure supplants the use, in some embodiments, of a bacterial strain with a relatively oxidizing cytoplasmic environment.

In particular embodiments, periplasmic expression of an ascorbate-dependent or ascorbate-analog-dependent biosynthetic enzyme such as prolyl-4-hydroxylase enables hydroxylation of a recombinantly expressed protein without concomitant expression of a sugar-1,4-lactone oxidase or sugar-1,4-lactone dehydrogenase, since ascorbate supplied in the growth medium can accumulate in the periplasm and can thus activate the periplasmically expressed biosynthetic enzyme.

In certain particular embodiments, the Gram-negative bacterial cells further comprise one or more nucleic acids encoding a peptide or protein to be hydroxylated, wherein the peptide or protein to be hydroxylated is expressed in the periplasmic space of the bacterial cell.

In further embodiments, the one or more nucleic acids encoding the enzyme comprise a first expression vector, and the one or more nucleic acids encoding the peptide or protein to be hydroxylated comprise a second expression vector, wherein each of the proteins encoded by each of the expression vectors is expressed in the cell comprising them.

In yet further embodiments, the nucleic acids encoding the enzyme and the peptide or protein to be hydroxylated comprise a single expression vector, wherein each of the proteins encoded by the expression vector is expressed in the cell comprising it.

In further embodiments of this aspect, the ascorbate-dependent biosynthetic enzyme is prolyl-4-hydroxylase, prolyl-3-hydroxylase, HIF prolyl hydroxylase, lysyl-5-hydroxylase, aspartyl beta-hydroxylase, asparaginyl beta-hydroxylase, or HIF asparaginyl hydroxylase, and in particular embodiments, the ascorbate-dependent biosynthetic enzyme is prolyl-4-hydroxylase, preferably human prolyl-4-hydroxylase.

In certain particular embodiments, the peptide or protein to be hydroxylated is collagen.

In various embodiments, the Gram-negative bacterial cells further comprise nucleic acids encoding a peptide or protein to be hydroxylated, wherein the peptide or protein to be hydroxylated is expressed in the periplasmic space of the bacterial cell.

In further embodiments, the nucleic acids encoding the enzyme comprise a first expression vector, and the nucleic acids encoding the peptide or protein to be hydroxylated comprise a second expression vector, wherein each of the proteins encoded by each of the expression vectors is expressed in the cell comprising them.

In yet further embodiments, the nucleic acids encoding the enzyme and the peptide or protein to be hydroxylated comprise a single expression vector, wherein each of the proteins encoded by the expression vector is expressed in the cell comprising it.

In still further embodiments, the peptide or protein to be hydroxylated is collagen.

In another aspect, the invention provides methods of making a post-translationally hydroxylated recombinant protein comprising the step of co-expressing in the periplasmic space of a Gram-negative bacterial cell nucleic acids encoding said protein and nucleic acids encoding an ascorbate-dependent or ascorbate-analog-dependent biosynthetic enzyme, and further comprising providing exogenous ascorbate or an exogenous ascorbate analog to the cell.

In particular embodiments, the nucleic acids encoding the ascorbate-dependent or ascorbate-analog-dependent biosynthetic enzyme comprise a first expression vector, and the nucleic acids encoding the protein comprise a second expression vector, wherein each of the proteins encoded by each of the expression vectors is expressed in the cell comprising them.

In further embodiments, the nucleic acids encoding the enzyme and the protein comprise a single expression vector, wherein each of the proteins encoded by the expression vector is expressed in the cell comprising it.

In further particular embodiments, the protein is collagen.

In another aspect, the invention provides post-translationally hydroxylated recombinant collagen molecules produced in a Gram-negative bacterial host cell co-expressing nucleic acids encoding said collagen molecules and one or more nucleic acids encoding an ascorbate-dependent biosynthetic enzyme.

In particular embodiments, the one or more nucleic acids encoding the ascorbate-dependent biosynthetic enzyme comprise a first expression vector, and the nucleic acids encoding the collagen molecule comprise a second expression vector, wherein each of the proteins encoded by each of the expression vectors is expressed in the cell comprising them.

In further embodiments, the nucleic acids encoding the ascorbate-dependent biosynthetic enzyme and the collagen molecule comprise a single expression vector, wherein each of the proteins encoded by the expression vector is expressed in the cell comprising it.

In particular embodiments, the one or more nucleic acids encoding the ascorbate-dependent biosynthetic enzyme comprise a first expression vector, and the nucleic acid encoding the protein comprises a second expression vector, wherein each of the proteins encoded by each of the expression vectors is expressed in the cell comprising them.

In further embodiments, the nucleic acids encoding the ascorbate-dependent biosynthetic enzyme and the protein comprise a single expression vector, wherein each of the proteins encoded by the expression vector is expressed in the cell comprising it.

In particular embodiments of this aspect, the ascorbate-dependent biosynthetic enzyme is prolyl-4-hydroxylase, prolyl-3-hydroxylase, HIF prolyl hydroxylase, lysyl-5-hydroxylase, aspartyl beta-hydroxylase, asparaginyl beta-hydroxylase, or HIF asparaginyl hydroxylase, and in particular embodiments, the ascorbate-dependent biosynthetic enzyme is prolyl-4-hydroxylase, preferably human prolyl-4-hydroxylase.

In yet further embodiments of this aspect, the ascorbate-dependent biosynthetic enzyme is prolyl-4-hydroxylase, prolyl-3-hydroxylase, HIF prolyl hydroxylase, lysyl-5-hydroxylase, aspartyl beta-hydroxylase, asparaginyl beta-hydroxylase, or HIF asparaginyl hydroxylase, and in particular embodiments, the ascorbate-dependent biosynthetic enzyme is prolyl-4-hydroxylase, preferably human prolyl-4-hydroxylase.

In another aspect, the invention provides kits for producing a post-translationally hydroxylated recombinant protein comprising a bacterial cell of the disclosure. The bacterial cells provided in said kits can be cells comprising one or more recombinant expression constructs encoding an ascorbate-dependent or ascorbate-analog-dependent biosynthetic enzyme and a sugar-1,4-lactone oxidase or sugar-1,4-lactone dehydrogenase. In alternative embodiments, the bacteria can additionally comprise one or more recombinant expression constructs encoding a protein to be post-translationally hydroxylated; in particular embodiments, the protein is collagen. In Gram-negative bacteria embodiments thereof, the cells can be cells comprising one or more recombinant expression constructs encoding an ascorbate-dependent or ascorbate-analog-dependent biosynthetic enzyme, and optionally can further comprise one or more recombinant expression constructs encoding a protein to be post-translationally hydroxylated; in particular embodiments, the protein is collagen. The kit can further contain instructions.

In some embodiments of any of the disclosed methods and products, the disclosed hydroxylated recombinant proteins comprise a collagenous domain that is sufficiently hydroxylated to form a triple-helical structure. Without any hydroxylation of collagen Y-position prolyl residues into 4-hydroxyproline, collagen chains will not properly or stably assemble into their triple-helical conformation at 37° C. If hydroxylation does not occur, the polypeptides remain non-helical, are poorly secreted by cells, and cannot self-assemble into collagen fibrils. Thus, in particular embodiments of the disclosed methods and products, the hydroxylated recombinant proteins comprise a collagenous domain, and an appropriate or sufficiently large number or fraction of Y-position prolyl residues within the collagenous domain are hydroxylated such that the collagenous domain forms a triple-helical structure.

In some embodiments of any of the aspects of the invention, the disclosed methods and products comprise a hydroxylated recombinant protein comprising a foldon domain of SEQ ID NO: 61. In certain embodiments of the disclosed methods and products, the foldon domain is fused to a terminus of the hydroxylated recombinant protein and facilitates self-assembly of the protein into a triple-helical structure.

As used herein, the “foldon domain” is the C-terminal domain of T4 fibritin, which is a triple-stranded coiled-coil protein that forms the “whiskers” of bacteriophage T4. The fibritin foldon domain serves as a registration motif that is both necessary and sufficient to promote the trimerization of fibritin. As such, it can be used as an artificial trimerization domain. The native structure of the foldon domain comprises a small, 27-residue trimeric β-hairpin propeller. It has been shown to successfully promote the trimerization of engineered protein systems such as short collagen fibers (Frank et al., 2001, J. Mol. Biol. 308: 1081-1089; Stetefeld et al., 2003, Structure 11: 339-346), HIV1 envelope glycoprotein (Yang et al., 2002, J. Virol. 76: 4634-4642), adenovirus fiber shaft (Papanikolopoulou et al., 2004, J. Biol. Chem. 279: 8991-8998; Papanikolopoulou et al., 2004, J. Mol. Biol. 342: 219-227), and rabies virus glycoprotein (Sissoeff et al., 2005, J. Gen. Virol. 86: 2543-2552). Fragments, truncations, and other variants of the foldon domain may be used in the disclosed methods and products.

In another aspect, the invention provides engineered bacterial cell-based systems capable of expressing recombinant proteins, for example hydroxylated recombinant proteins, comprising:
a. one or more nucleic acids encoding a sugar-1,4-lactone oxidase or a sugar-1,4-lactone dehydrogenase; and
b. one or more nucleic acids encoding an ascorbate-dependent biosynthetic enzyme,
   wherein the nucleic acids are either genes inserted into the bacterial genome or plasmids.

The expression vectors of the disclosure are introduced into the bacterial host cells by any method known to the art, such as calcium chloride-mediated transfection, electroporation or otherwise. After transfection, host cells comprising the expression vector or vectors can be selected on the basis of one or more selectable markers that are included in the expression vector(s).

As will be apparent to one skilled in the art, the particulars of the selection process depend on the identities of the selectable markers. If a selectable marker is an antibiotic resistance gene, the transfected host cell population can be cultured in the presence of an antibiotic to which resistance is conferred by the selectable marker. The antibiotic kills or inhibits the growth of those cells that do not carry the resistance gene, and permit proliferation of those host cells that carry the resistance gene and the associated expression construct. If a selectable marker is a gene which complements an auxotrophy of the host cells, the transfected host cell population can be cultivated in the absence of the compound for which the host cells are auxotrophic. The cells that carry the complementing gene can be able to proliferate under such growth conditions and can also presumably carry the rest of the expression construct.

After selection, host cells can be cloned according to any appropriate method known in the art. For example, microbial host cells can be plated on solid media under selection conditions, after which single clones can be selected for further selection, characterization, or use. This process can be repeated one or more times to enhance the stability of the expression construct within the host cell. To produce collagen or another hydroxylated recombinant protein of interest, recombinant host cells comprising one or more expression vectors can be cultured to expand cell numbers in any appropriate culturing apparatus known in the art, such as a shaken culture flask or a fermenter.

The culture medium used to culture recombinant bacterial cells will depend on the identity of the bacteria. Culture media used for various recombinant host cells are well known in the art. The culture medium generally comprises inorganic salts and compounds, amino acids, carbohydrates, vitamins and other compounds which are either necessary for the growth of the host cells or which improve the health and/or growth of the host cells.

If the bacterial host cells are Gram-negative bacterial cells and comprise a recombinant ascorbate-dependent or ascorbate-analog-dependent biosynthetic enzyme, such as prolyl-4-hydroxylase, which is expressed in the periplasmic space of the bacteria, then vitamin C (ascorbic acid or one of its salts) or an ascorbate analog can be added to the culture medium. If ascorbic acid is added, it is generally added to a concentration of between 0.05 mM to 20 mM, preferably to a concentration of around 2 mM.

Iron(II) is a necessary co-factor for some ascorbate-dependent biosynthetic enzymes, such as prolyl-4-hydroxylase. Iron(II) concentrations in growth media for proper functioning of prolyl-4-hydroxylase range from about 0.05 mM to 1 mM, and are preferably at around 0.5 mM. Many types of growth media contain enough iron(II) for proper functioning of the hydroxylase, such that iron(II) need not be added. In cases where the iron(II) concentration is lower than required for proper functioning of the hydroxylase, the media should be supplemented with iron(II).

After expression, the particular method of recovery of collagen from the culture will depend on the host cell type and the expression construct. Collagen can be trapped in the cytoplasm, and in particular embodiments, collagen can be trapped in the periplasm. Cell walls can be removed or weakened to release collagen located in the cytoplasm or periplasm. Disruption can be accomplished by any means known in the art, including sonication, microfluidization, lysis in a French press or similar apparatus, or disruption by vigorous agitation/milling with glass beads. Lysis or disruption of recombinant host cells is preferably carried out in a buffer of sufficient ionic strength to allow the collagen to remain in soluble form (e.g., more than 0.1 M NaCl, and less than 4.0 M total salts including the buffer).

Recovered collagen can be purified using known techniques, where the particular technique used depends on the host cell type and the expression construct. Generally, recovered collagen solutions are first clarified (if the collagen is recovered by cell disruption or lysis). Clarification is generally accomplished by centrifugation, but can also be accomplished by sedimentation and/or filtration if desired. In cases where the collagen-containing solution contains a substantial lipid content (for example, when the collagen has been recovered by cellular lysis or disruption), the solution can also be delipidated. Delipidation can be accomplished by the use of an adsorbant such as diatomaceous earth or diatomite such as that sold as CELITE™ 512 (AdvancedMinerals). When diatomaceous earth or diatomite is used for delipidation, it is preferably prewashed before use, then removed after use by filtration.

The collagen product can be further purified by any one or more purification techniques known in the art, including gel filtration chromatography, ion exchange chromatography (for example, cation exchange chromatography can be used to adsorb the collagen to the matrix, and anion exchange chromatography can be used to remove a contaminant from a collagen-containing solution), affinity chromatography, hydrophobic interaction chromatography, and high-performance liquid chromatography. Additionally, collagen solubility can be manipulated by alterations in the pH or ionic strength of the buffer. In particular, any one of the following manipulations can be used, singly or in combination with others to purify products of the disclosure: insolubilize collagen in low ionic strength buffers; precipitate collagen at high ionic strengths; dissolve collagen in acidic solutions; and form collagen fibrils (by assembly of trimeric monomers) in low ionic strength buffers near neutral pH (i.e., about pH 6 to 8), thereby eliminating proteins that do not precipitate at high ionic strength.

Recovered or purified collagen can also be treated to produce gelatin by any technique known in the art, including thermal denaturation, acid treatment, alkali treatment, or any combination thereof.

After purification, collagen produced according to the invention can be modified by crosslinking in order to stabilize the collagen triple helix, thereby improving the resistance of trimeric fibrillar collagen to thermal denaturation and proteolytic degradation. Methods for crosslinking collagen are known in the art. In general, the collagen is resuspended in a buffered solution such as phosphate buffered saline at about 3 mg/mL, and mixed with a relatively low concentration of glutaraldehyde, preferably about 0.0025-1% (v/v). The collagen/glutaraldehyde mixture is then incubated to allow crosslinking to occur, preferably at a temperature below room temperature (i.e., less than about 20° C.). For crosslinking, the glutaraldehyde is preferably of high purity and contains relatively low amounts of glutaraldehyde polymer.

In certain embodiments of the disclosed methods and products, one or more of the nucleic acids encoding the sugar-1,4-lactone oxidase, the ascorbate-dependent biosynthetic enzyme, and the peptide or protein to be hydroxylated are incorporated into the bacterial chromosome. Methods of incorporating nucleic acids into the bacterial chromosome are known in the art. For example, the nucleic acids of the disclosure may be incorporated into a bacteriophage A vector, which may then integrate itself into the host cell's chromosome (see, for example, Sieg et al. (1989), “A versatile phage lambda expression vector system for cloning in Escherichia coli.” Gene 75(2): 261-70.). Alternatively, the nucleic acids of the disclosure may be placed into a gene cassette under the control of a promoter that is suitable for inserting a gene into the chromosome of a bacterium, such as the very strong bacteriophage A promoter left (PL), as disclosed in International Publication No. WO 2006/029449.

The methods and products of the disclosure can be used for a wide variety of pharmaceutical, cosmetic, and medicinal purposes that are known in the art, including as a component in artificial skin (see, for example, U.S. Pat. No. 5,800,811, herein incorporated by reference in its entirety), alone or in combination with antibiotics in a dressing to promote wound healing (see, for example, U.S. Pat. No. 5,219,576), or as a component in cardiac devices (see, for example, U.S. Pat. No. 7,008,397).

### EXAMPLES

The Examples that follow are illustrative of specific embodiments of the invention, and various uses thereof. They are set forth for explanatory purposes only, and are not to be taken as limiting the invention.

### Example 1

### Expression and Purification of GST-(PPG)₅ Without ALO1 co-Expression

GST-(PPG)₅ (SEQ ID NO: 23; GST is glutathione S-transferase) was cloned into a pCOLADuet vector as follows. An oligonucleotide encoding (PPG)₅ (SEQ ID NO: 24) with a BamHI restriction site at the 5′ end and an XhoI site at the 3′ end with the sequence:

(SEQ ID NO: 25)
GCTAGGATCCCCGCCGGGTCCGCCAGGCCCACCGGGTCCACCTGGCCCG
CCTGGTTAAAGGAGAAGCAGGTGCTGGACAGGGCGAGGCATCGTGCCTG
GTTAACTCGAGCTAG

was amplified using PCR to obtain a specific double-stranded DNA using primers GCTAGGATCC CCGCCGGGTC (SEQ ID NO: 26) and CTAGCTCGAG TTAACCAGGC (SEQ ID NO: 27) and the following PCR conditions: (1) denature for 5 min at 95° C.; (2) denature for 1 min at 95° C.; (3) anneal for 1 min at 55° C.; (4) elongation for 1 min at 72° C.; (5) repeat steps (2)-(4) 30 times; (6) elongation for 10 min at 72° C.; hold at 4° C. The specific double-stranded DNA was then digested by BamHI and XhoI restriction endonucleases and inserted into pGEX4T-1 (GE Healthcare) in order to create a fusion of (PPG)₅ and glutathione S-transferase (GST) with an intervening thrombin protease cleavage (Novagen, Inc., San Diego, Calif.) site, termed herein GST-(PPG)₅. The pGEX-4T-1 vector map was obtained from GE Healthcare's website, last accessed Apr. 2, 2010. DNA encoding GST-(PPG)₅ was isolated from pGEX4T-1 by PCR using primers (forward primer: CAGCTACCAT GGGTtcccct atactaggtt attggaaaat taagggcc (SEQ ID NO: 28); reverse primer: CCTGACGGGC TTGTCTGCTC CC (SEQ ID NO: 29)) that flanked regions on the 5′ side of the translation initiation codon including an NcoI site and the 3′ side of the stop codon including a NotI site. The PCR fragment was digested with NcoI and NotI restriction enzymes (New England Biolabs, MA) by adding 2 units enzyme for each μg DNA, and incubating at 37° C. for 2 hours. After digestion, the DNA was separated in an agarose gel, the expected band was extracted and purified by QIAquick Gel Extraction Kit (Qiagen, Valencia, Calif.), and the resulting amplified sequence was ligated into the first cloning site in the multiple cloning site (MCS) of the pCOLADuet-1 vector (Novagen, Inc., San Diego, Calif.), yielding the plasmid pSD1000. pSD1000 was transformed into Origami 2 cells (Novagen, Inc.) with and without human prolyl-4-hydroxylase (P4H) co-transformation. The pCOLADuet-1 vector map was obtained from Novagen, Inc.'s website, last accessed Apr. 2, 2010.

A pET22b vector (Novagen, Inc., San Diego, Calif.), designated pBK1.PDI1.P4H7, was used to express human P4H, as described by Kersteen et al. (2004, Protein Purification and Expression 38: 279-291). The pET22b vector map was obtained from Novagen, Inc.'s web site last accessed Apr. 2, 2010. Briefly, cDNAs encoding the α and β subunits of human prolyl-4-hydroxylase were cloned into the same plasmid. From this bicistronic vector, both cDNAs were able to be transcribed from the same T7 promoter, with each subunit having its own ribosome binding site (rbs) for translation initiation. cDNA encoding human protein disulfide isomerase (PDI—the β subunit of P4H), without its signal sequence encoding region, was inserted between the NdeI and BamHI restriction sites of a pET22b(+) expression vector (Novagen, Inc., San Diego, Calif.) to give a plasmid, designated pBK1.PDI1. The pET22b(+) vector map was obtained from Novagen, Inc.'s website, last accessed Apr. 2, 2010.

cDNA encoding P4Hα(I) subunit (PA11 clone), was isolated from HeLa cells and inserted into a pBSKS vector (pBS.LF17-1). The pBSKS vector map was obtained from Addgene's web site last accessed Apr. 2, 2010. DNA encoding P4Hα(I) was isolated from the pBS.LF17-1 vector by PCR using primers that flank regions on the 5′ side of the translation initiation codon and the 3′ side of the stop codon, each of which includes a BamHI restriction site. The resulting PCR fragment was cloned into a PCR4-TOPO vector (Invitrogen, Carlsbad, Calif.), digested with BamHI, and then ligated into the pBK1.PDI1 plasmid, which was previously digested with BamHI, yielding plasmid pBK1.PDI1.P4H1. The PCR4-TOPO vector map was obtained from Invitrogen's website, last accessed Apr. 2, 2010.

A site-directed mutagenesis kit (QuikChange, Stratagene, La Jolla, Calif.) was used to remove DNA encoding the signal sequence of P4Hα(I) according to the manufacturer's protocols. The resulting plasmid pBK1.PDI1.P4H5 produced the P4Hα(I)235-534/β enzyme (a P4H oligomer with a 32 kDa α subunit).

A plasmid encoding the P4Hα(I) subunit alone was produced by digesting pBK1.PDI1.P4H5 with NdeI, removing the DNA fragment encoding PDI, and then ligating the vector. QuikChange mutagenesis was then applied to the resulting construct (pBK1.P4H5) to add a BamHI site to the 5′ end of the pET22b(+) rbs, yielding plasmid pBK1.P4H6. BamHI digestion of the pBK1.PDI1 and pBK1.P4H6 plasmids, followed by ligation, resulted in a vector, designated pBK1.PDI1.P4H6, with the PDI cDNA preceding the P4Hα(I) cDNA, both having the polylinker of pET22b(+) on the 5′ side of their start codons. Finally, the ATG codon of Met235 of the α subunit was replaced with a CTT codon (leucine) by QuikChange mutagenesis to yield plasmid pBK1.PDI1.P4H7, which was used to produce the full-length P4H tetramer.

For co-transformation, 1 μL of pCOLADuet-1 vector that contained the GST-(PPG)₅ gene in the first MCS and 1 μL pBKI.PDI1.P4H7 (encoding P4Hα(I)) were added to a 20 μL aliquot of Origami 2 (DE3) competent cells at the same time. The cells were placed on ice for 30 minutes, heat shocked at 42° C. for 1 min, and then put back on ice for 2 minutes. After adding 300 μL SOC (Super Optimal broth with Catabolite repression) media, the cells were shaken at 200 rpm at 37° C. for 1 hour before plating on an LB (Luria-Bertani) agar plate containing 30 μg/mL kanamycin and 200 μg/mL ampicillin; the plate was incubated at 37° C. overnight.

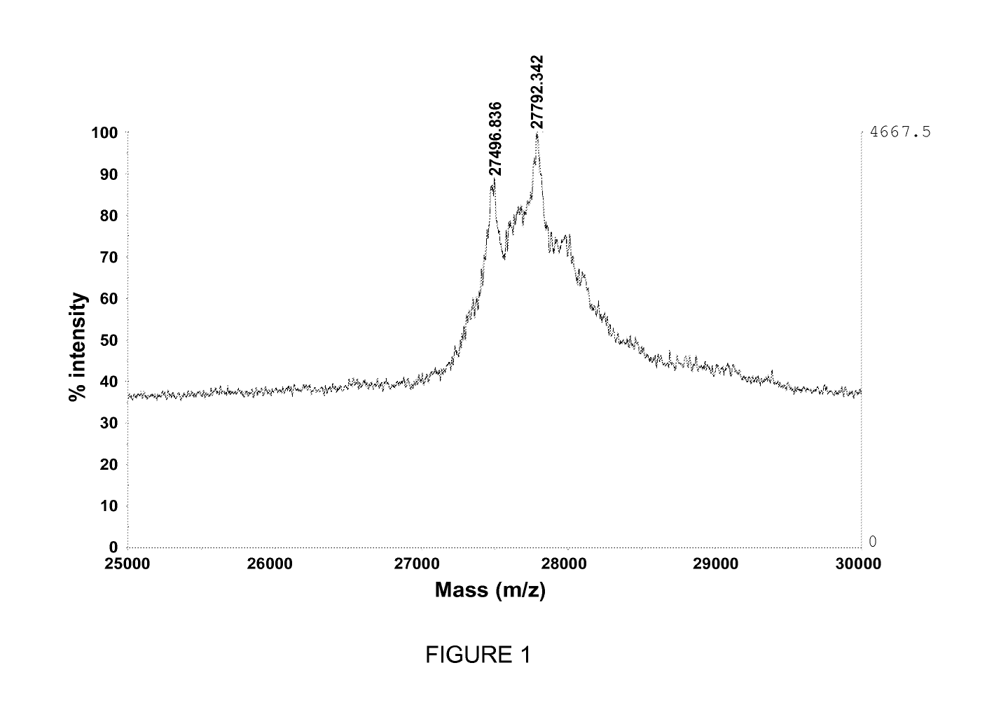

After 2-liter shaker expressions in terrific broth, the proteins were purified by glutathione agarose resin. The proteins appeared in a single band by SDS-PAGE. The molecular weight of GST-(PPG)₅ (SEQ ID NO: 23) without P4H co-expression was confirmed by MALDI mass spectrometry. As shown in FIG. 1, the first peak is consistent with the expected molecular weight (˜27.5 kD), while the second peak is GST-(PPG)₅ plus a glutathione adduct (glutathione was not removed from the protein buffer before MALDI). As expected, there was no hydroxylation observed even with P4H co-expression as the cytosol of E. coli does not provide the necessary cofactors necessary for hydroxylase activity.

To confirm that P4H can hydroxylate the substrate GST-(PPG)₅ (SEQ ID NO: 23), and to develop a method for detecting GST-(PPG)₅ hydroxylation, 0.2 mg of GST-(PPG)₅ was incubated in 100 μL of 50 mM Tris-HCl buffer, pH 7.8 containing bovine serum albumin (1 mg/mL), catalase (100 μg/mL), dithiothreitol (100 μM), Fe(II)SO₄ (50 μM), α-ketoglutarate (500 μM), and ascorbate (2 mM), and an aliquot of purified P4H (50 μL at 4.5 μM) was added to the mixture. P4H was prepared recombinantly in E. coli and purified by polyproline affinity chromatography followed by ion exchange chromatography. A positive control, wherein the 4-residue peptide Ac-GFPG-NH₂ (SEQ ID NO: 30), previously shown to be capable of hydroxylation by P4H, was used as a substrate, rather than GST-(PPG)₅, was included in these experiments. The negative control was GST-(PPG)₅ incubated in buffer without P4H enzyme. The reactions took place for 2 hours at 37° C. The positive control was boiled for 5 min to precipitate the proteins, and the peptide was recovered in the supernatant after centrifugation. The samples with GST-(PPG)₅ (0.1 mg/mL) were then incubated with a 5-fold excess of thrombin in Dulbecco's phosphate-buffered saline (DPBS) to cleave GST:

(SEQ ID NO: 31)
MSPILGYWKIKGLVQPTRLLLEYLEEKYEEHLYERDEGDKWRNKKFELG
LEFPNLPYYIDGDVKLTQSMAIIRYIADKHNMLGGCPKERAEISMLEGA
VLDIRYGVSRIAYSKDFETLKVDFLSKLPEMLKMFEDRLCHKTYLNGDH
VTHPDFMLYDALDVVLYMDPMCLDAFPKLVCFKKRIEAIPQIDKYLKSS
KYIAWPLQGWQATFGGGDHPPKSDLVPR

from the GS-(PPG)₅ peptide (SEQ ID NO: 32). After 2 hour cleavage, the cleaved peptide was separated from GST and P4H by running the cleavage mixture through a 10 kD cutoff spin concentrator (Millipore).

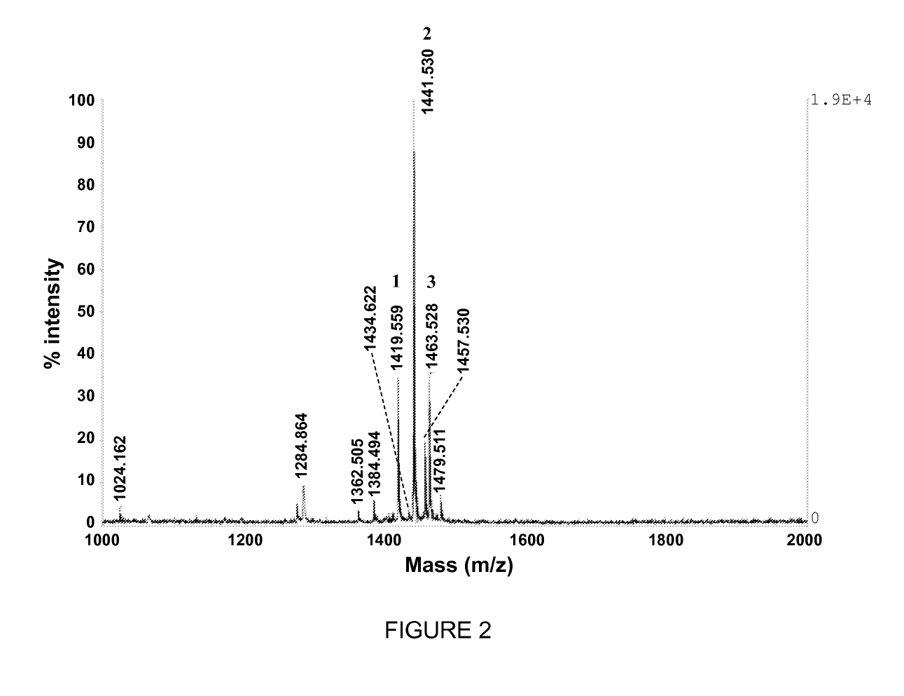

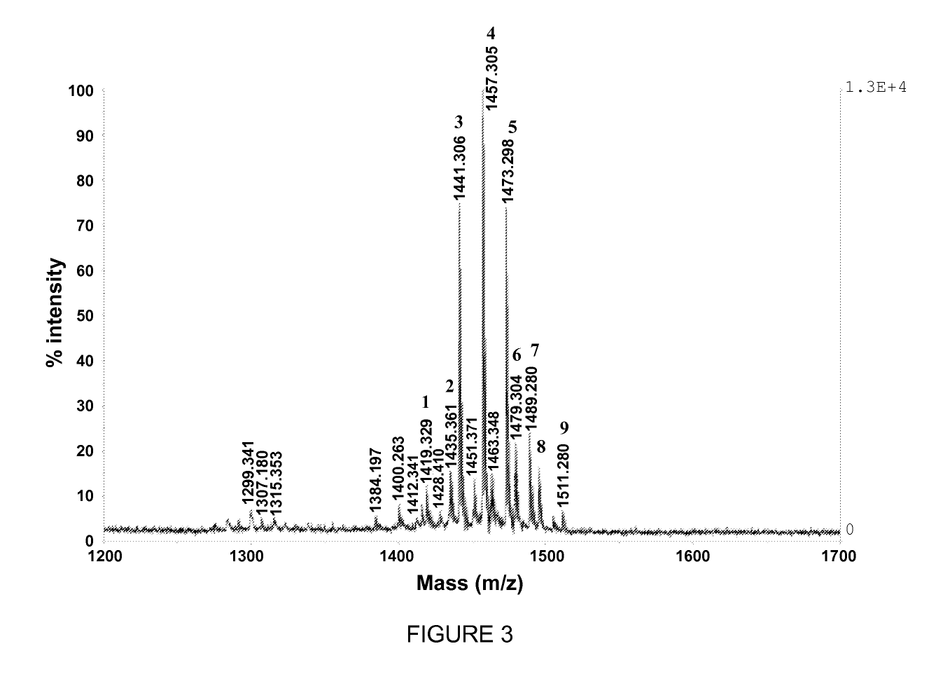

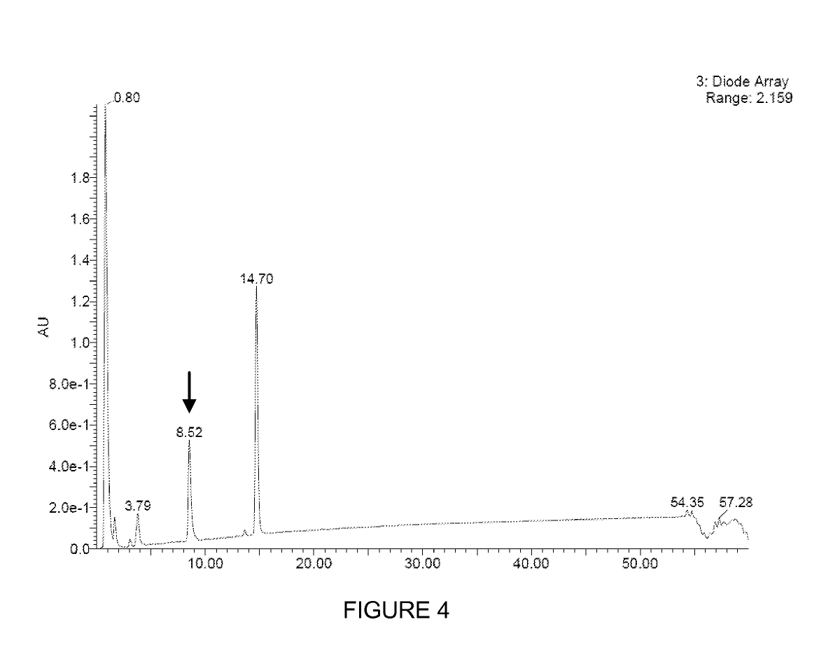

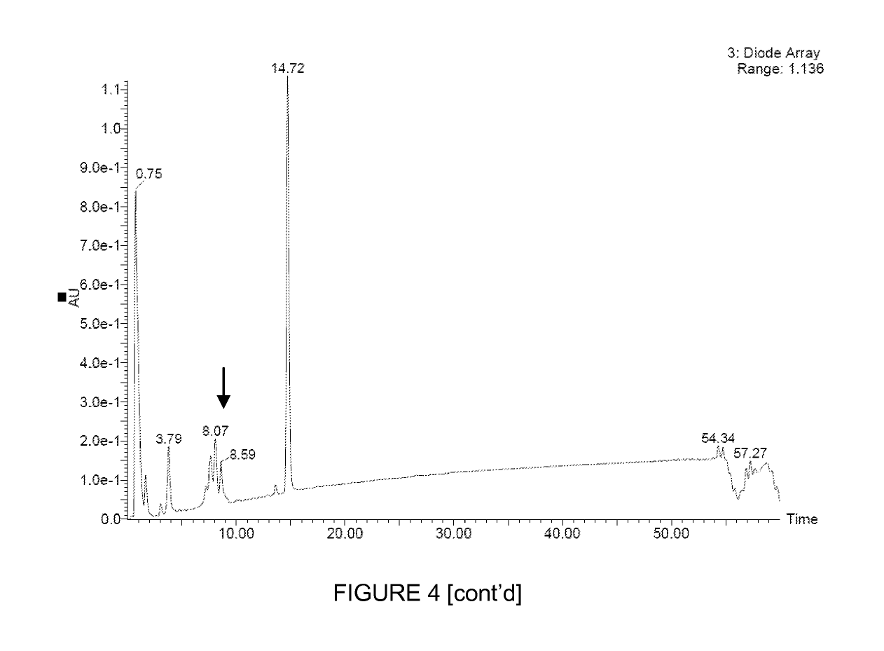

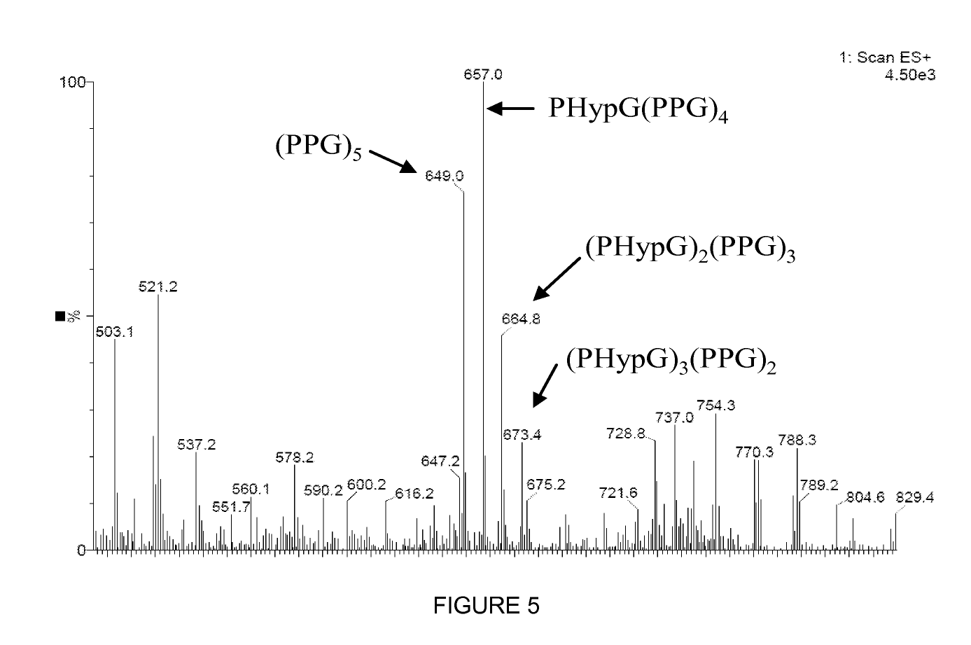

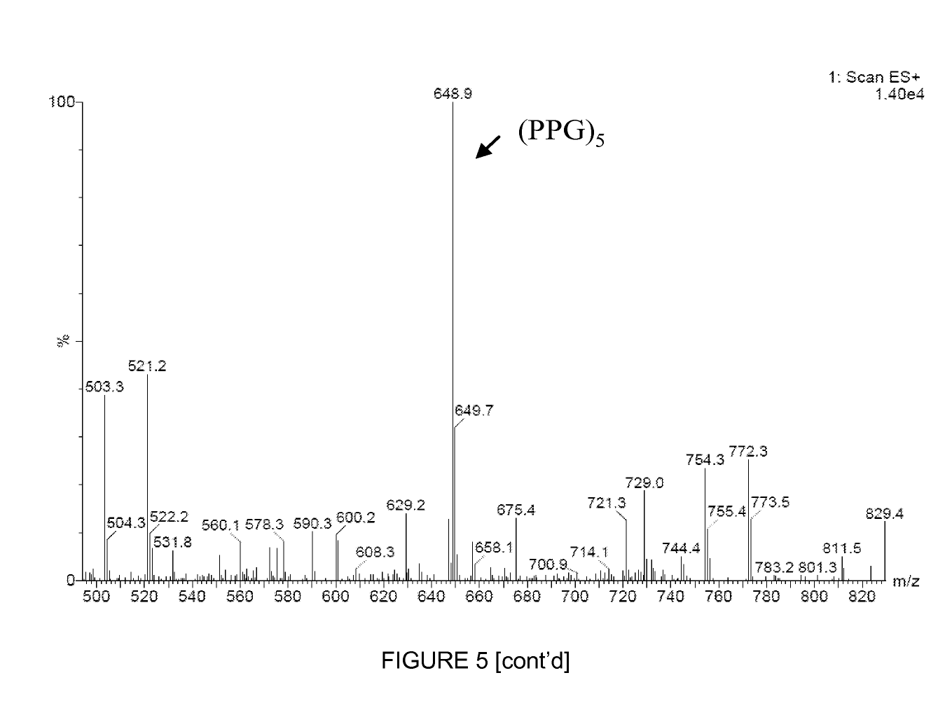

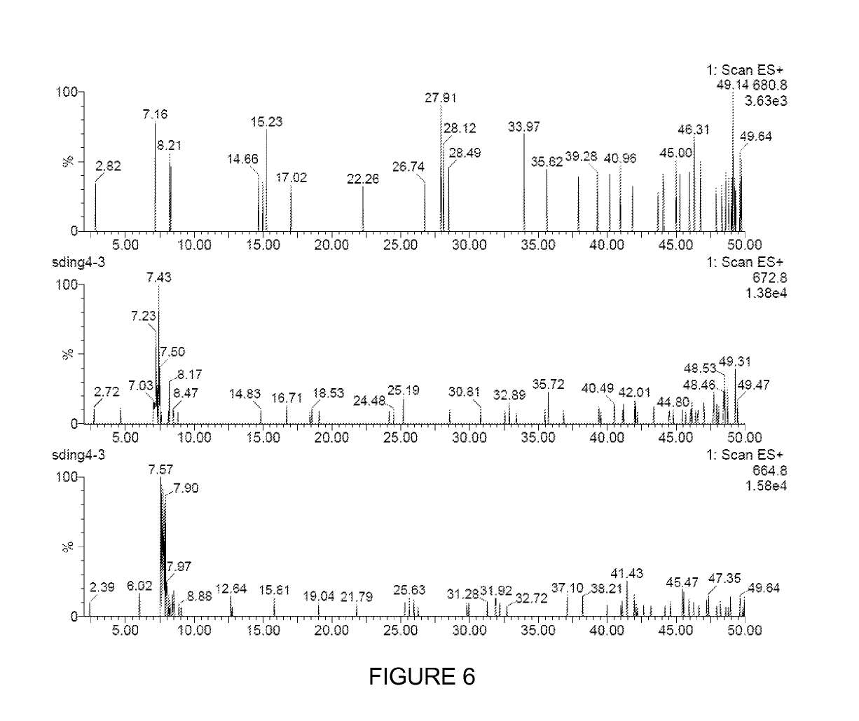

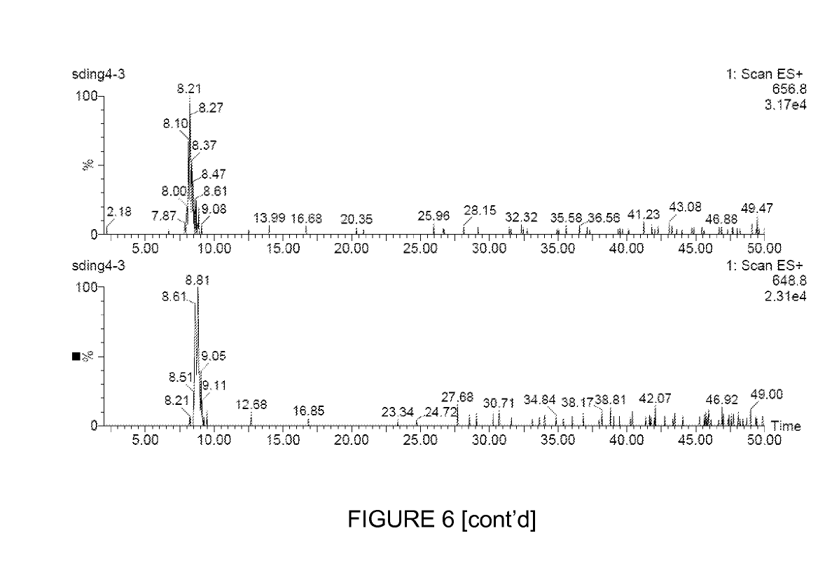

Samples were then mixed with sinapinic acid (10 mg/mL, in 50% acetonitrile solution containing 0.1% trifluoroacetic acid) and analyzed by MALDI-TOF mass spectrometry (Applied Biosystems, CA) (FIGS. 2 and 3). In addition, samples were analyzed by injection into a Micromass ZQ LC-MS (FIGS. 4 and 5) using a gradient of 0-100% acetonitrile with 0.1% formic acid; UV absorbance was monitored at 214 nm. The positive control showed that the proline residue in Ac-GFPG-NH₂ was hydroxylated.

The calculated molecular weight of the unhydroxylated (PPG)₅ peptide after cleavage (GSPPGPPGPPGPPGPPG, GS-(PPG)₅; SEQ ID NO: 32) was 1417.7 (monoisotopic mass in Da). As shown in FIG. 3, peaks having an apparent molecular weight of 1441, 1457, and 1471 were detected, indicating that up to 3 hydroxyproline residues were produced in the (PPG)₅ peptide after incubation with P4H.

The LC-MS results are consistent with the MALDI results (FIGS. 4 and 5). The species with a retention time of 8.5 min was GS-(PPG)₅ and peaks having slightly shorter retention times were the hydroxylated species. The doubly-charged species of hydroxylated compounds exhibited an additional m/z=8. The observed m/z corresponds to (PPG)₅ (SEQ ID NO: 24), rather than GS-(PPG)₅ (SEQ ID NO: 32), indicating that the N-terminal GS residues were cleaved during analysis, most likely during ionization. These experiments confirmed that P4H is able to hydroxylate GST-(PPG)₅ (SEQ ID NO: 23).

### Example 2

### Assessment of Exogenous Ascorbate as a Carbon Source

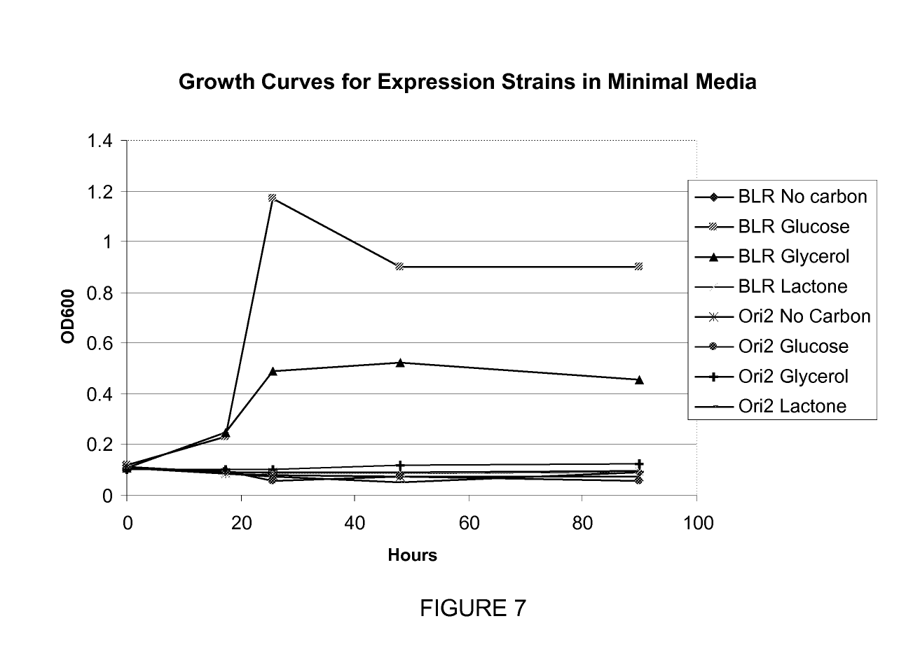

To determine if expression strains BLR (DE3) (Novagen, Inc.) and Origami 2 (Novagen, Inc.) were able to assimilate ascorbate as a carbon source, each strain was grown in M9 minimal media supplemented with 0.2 wt % of glucose, glycerol, or lactone (FIG. 7). Although the BLR strain grew on some carbon sources, the Origami 2 strain did not grow in M9 minimal media, even with glucose supplementation.

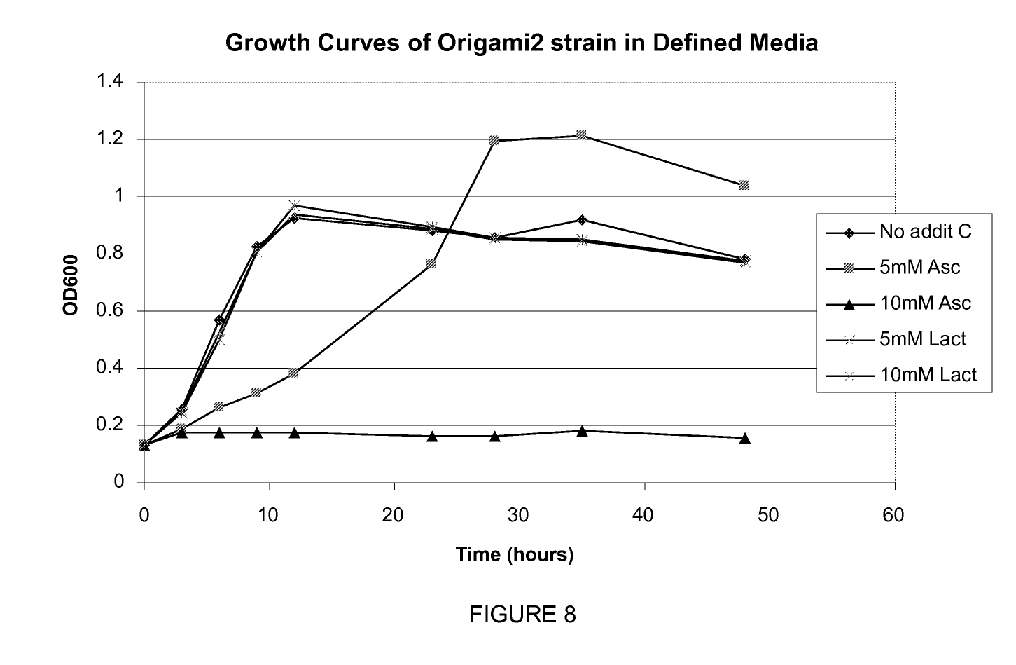

Origami 2 cells were able to grow on M9 supplemented with 10 mL of 100× vitamin stock solution (0.42 g/L riboflavin, 5.4 g/L pantothenic acid, 6 g/L niacin, 1.4 g/L pyridoxine, 0.06 g/L biotin, and 0.04 g/L folic acid) per 1000 mL, 10 mL of 100× trace metal stock solution (27 g/L FeCl₃.6H₂O, 2 g/L ZnCl₂.4H₂O, 2 g/L CaCl₂.6H₂O, 2 g/L Na₂MoO₄.2H₂O, 1.9 g/L CuSO₄.5H₂O, 0.5 g/L H₃BO₃, and 100 mL/L concentrated HCl) per 1000 mL, and 0.4% casamino acids (FIG. 8). Additionally, the eventual overgrowth of the Origami 2 strain with the addition of 5 mM ascorbate (FIG. 8) strongly suggested that these bacteria could assimilate carbon from ascorbate, although the ascorbate shows a dose-dependent toxicity. On the other hand, L-gulono-1,4-lactone was not a viable carbon source, which indicated that it was either not transported into the cell, or not metabolized there.

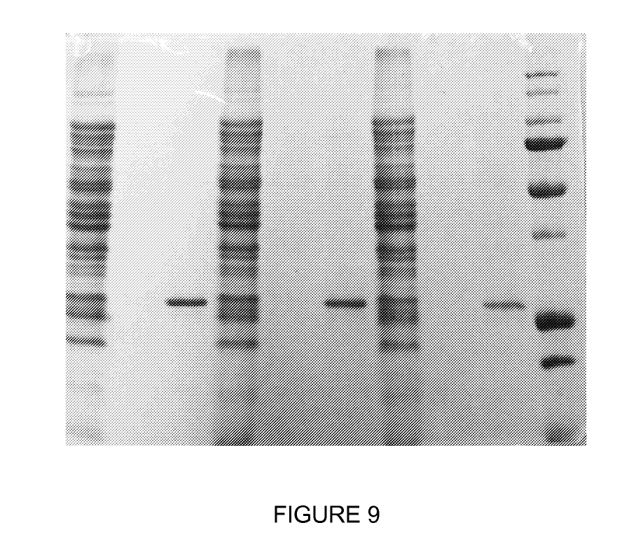

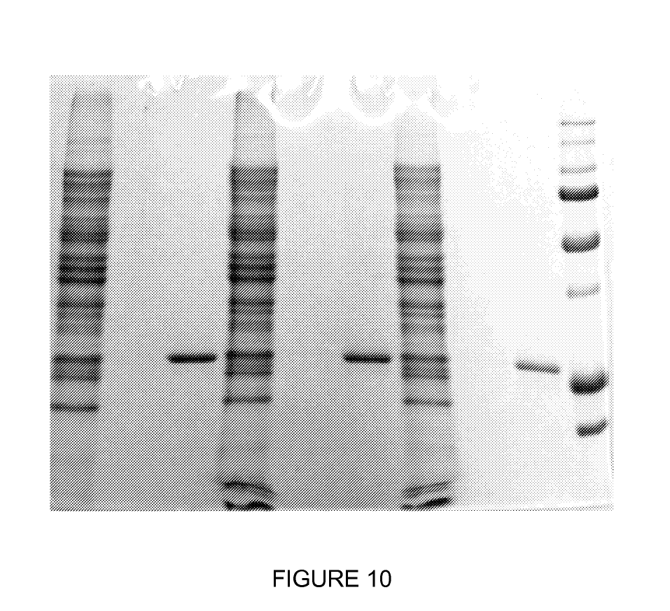

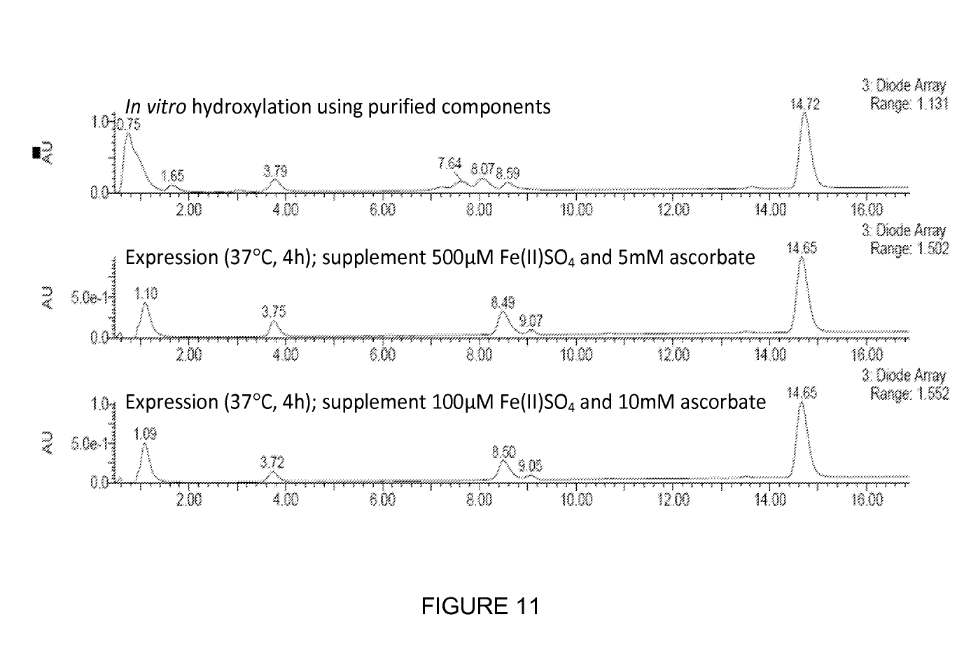

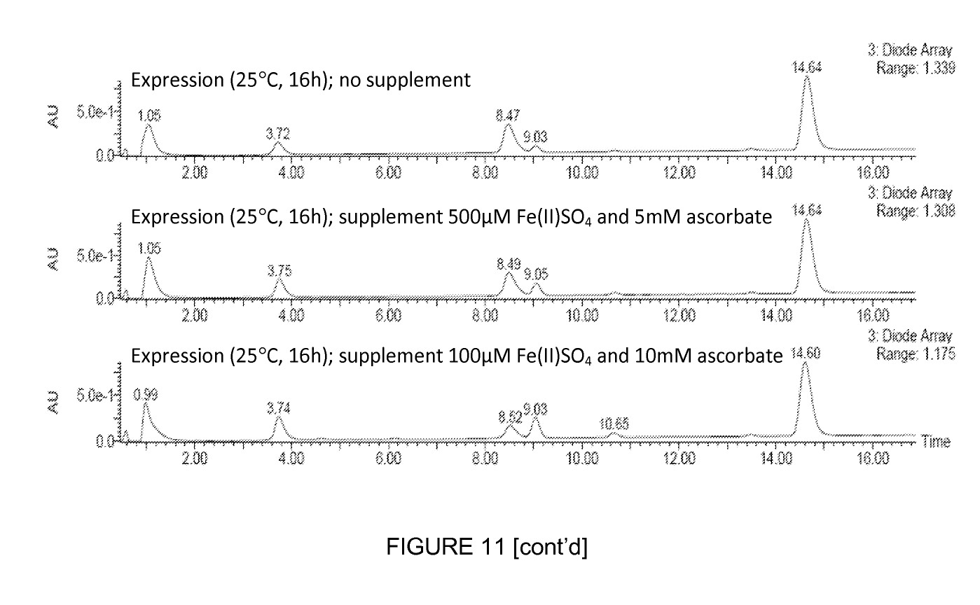

To determine whether Origami 2 cells were able to accumulate significant intracellular levels of exogenously supplied ascorbate, and thereby activate P4H intracellularly, Origami 2 cells were transformed with the genes for P4H and GST-(PPG)₅ and then grown at 37° C. to a concentration having an OD₆₀₀=0.6 in M9 minimal media supplemented with vitamins, minerals, casamino acids, and 5 mM ascorbate. After induction of protein production with IPTG, the media was supplemented with different levels of Fe(II)SO₄/ascorbate. GST-(PPG)₅ samples were purified as described above, and all samples after purification yielded a single band by SDS PAGE (FIG. 9). As demonstrated by the chromatograms in FIG. 10, none of the above experimental conditions yielded hydroxylated (PPG)₅. This result is in agreement with expectations as E. coli is known not to transport ascorbate into the cytoplasm under aerobic conditions. As molecular oxygen is a required substrate for P4H, this experiment confirms that exogenous addition of ascorbic acid to cells that express P4H intracellularly was not a viable method of achieving hydroxylation.

### Example 3

### Construction of Expression Plasmids Comprising ALO1 DNA

An oligonucleotide encoding (PPG)₅ with a BamHI restriction site at the 5′ end and an XhoI site at the 3′ end with the sequence:

(SEQ ID NO: 25)
GCTAGGATCCCCGCCGGGTCCGCCAGGCCCACCGGGTCCACCTGGCCCG
CCTGGTTAAAGGAGAAGCAGGTGCTGGACAGGGCGAGGCATCGTGCCTG
GTTAACTCGAGCTAG

was amplified using PCR to obtain a specific double-stranded DNA using primers GCTAGGATCCCCGCCGGGTC (SEQ ID NO: 26) and CTAGCTCGAGTTAACCAGGC (SEQ ID NO: 27) and the following PCR conditions: (1) denature for 5 min at 95° C.; (2) denature for 1 min at 95° C.; (3) anneal for 1 min at 55° C.; (4) elongation for 1 min at 72° C.; (5) repeat steps (2)-(4) 30 times; (6) elongation for 10 min at 72° C.; hold at 4° C. The specific double-stranded DNA was then digested by BamHI and XhoI restriction endonucleases (New England Biolabs, MA) by adding 2 units enzyme for each μg DNA, and incubating at 37° C. for 2 hours. After digestion, the DNA was separated in an agarose gel, the expected band was extracted and purified by QIAquick Gel Extraction Kit (Qiagen, Valencia, Calif.), and the resulting amplified sequence was inserted into pGEX4T-1 in order to create a fusion of (PPG)₅ and glutathione S-transferase (GST) with an intervening thrombin protease cleavage site, termed GST-(PPG)₅ herein. DNA encoding GST-(PPG)₅ was isolated from pGEX4T-1 by PCR using primers (SEQ ID NOS: 28-29) that flanked regions on the 5′ side of the translation initiation codon including an NcoI site and the 3′ side of the stop codon including a NotI site. The PCR fragment was digested with NcoI and NotI restriction enzymes (New England Biolabs, MA) by adding 2 units enzyme for each μg DNA, and incubating at 37° C. for 2 hours. After digestion, the DNA was separated in an agarose gel, the expected band was extracted and purified by QIAquick Gel Extraction Kit (Qiagen, CA), and the resulting amplified sequence was ligated into the first cloning site in the multiple cloning site (MCS) of the pCOLADuet-1 vector (Novagen, Inc.), yielding the plasmid pSD1000.

cDNA encoding the ALO1 gene of Saccharomyces cerevisiae (strain EBY100) was amplified from genomic DNA as previously described (Lee et al., 1999, Appl. Environ. Microbiol. 65: 4685-7) (see SEQ ID NO: 1 for an exemplary S. cerevisiae ALO1 coding sequence). Briefly, oligonucleotide primers were synthesized on the basis of the nucleotide sequence of the ALO1 gene with the sequences 5′-TTTCACCATATGTCTACTATCC-3′ (forward primer; SEQ ID NO: 33) and 5′-AAGGATCCTAGTCGGACAACTC-3′ (reverse primer; SEQ ID NO: 34). The primers were designed so that the amplified DNA contained the entire open reading frame of the ALO1 gene with a NdeI site at the 5′ end and a BamHI site at the 3′ end. PCR was carried out with Pfu Turbo Hotstart DNA polymerase (Stratagene). Template genomic DNA for PCR was prepared from S. cerevisiae ATCC 44774 according to established methods (Wach et al., 1994, “Procedures for isolating yeast DNA for different purposes,” in J. R. Johnston (ed.), MOLECULAR GENETICS OF YEASTS, IRL Press: Oxford, pp. 1-16). The reaction mixture contained 0.5 mM each forward and reverse primer, 0.2 mM deoxynucleoside triphosphate, 2.0 mM MgSO₄, 1×PCR buffer, and 0.5 mg of template genomic DNA and 2.5 U Pfu polymerase per 50 mL. The mixture was subjected to 30 cycles of 1 min denaturation at 94° C., 1 min annealing at 50° C., and 2 min extension at 72° C.

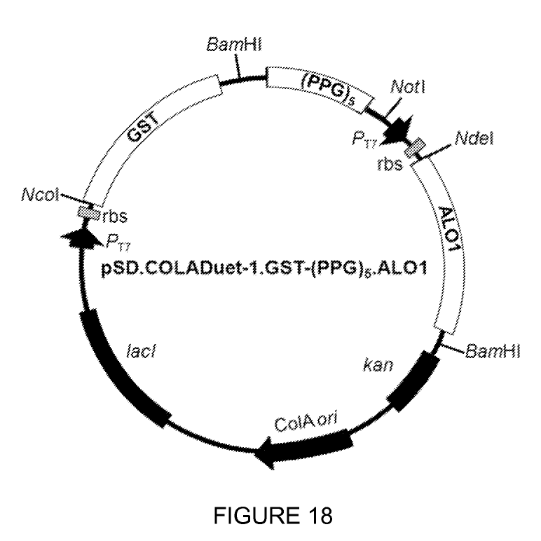

The PCR fragment obtained as described above was inserted into a pET19b vector (Novagen, Inc.), and then isolated from the vector by PCR using forward primer: CCCGAAAGGA AGCTCGAGTT GGCTGCTG (SEQ ID NO: 35) and reverse primer: CAGCAGCCAA CTCGAGCTTC CTTTCGGG (SEQ ID NO: 36) that introduced an XhoI site on the 3′ side of the stop codon and retained the NdeI site on the 5′ side. The pET19b vector map was obtained from Novagen, Inc.'s web site last accessed Apr. 2, 2010. At the same time, a site directed mutagenesis PCR was carried out on plasmid pSD1000 to remove the XhoI site from the end of (PPG)₅ using forward primer CGTGCCTGGT TAACTGAGCG GCCGCATAATG (SEQ ID NO: 37) and reverse primer CATTATGCGG CCGCTCAGTT AACCAGGCACG (SEQ ID NO: 38). The ALO1 fragment was digested by NdeI and XhoI restriction enzymes, and inserted into the second cloning site of the mutated plasmid pSD1000. The result was a pCOLADuet-1 vector that contained the GST-(PPG)₅ gene in the first MCS, and ALO1 in the second MCS designated pSD1001 (the plasmid map is shown in FIG. 18).

### Example 4

### Protein Expression, Purification, and Characterization

pSD1001 (pCOLADuet-1 vector that contained the GST-(PPG)₅ gene in the first MCS, and ALO1 in the second MCS) was co-transformed with pBKI.PDI1.P4H7 (encoding P4Hα(I), as described in Example 1) into Origami 2 (DE3) competent cells. For co-transformation, 1 μL of pCOLADuet-1 vector that contained the GST-(PPG)₅ gene in the first MCS and 1 μL pBKI.PDI1.P4H7 (encoding P4Hα(I)) were added to a 20 μL aliquot of Origami 2 (DE3) competent cells at the same time. The cells were placed on ice for 30 minutes, heat shocked at 42° C. for 1 min, and then put back on ice for 2 minutes. After adding 300 μL SOC (Super Optimal broth with Catabolite repression) media, the cells were shaken at 200 rpm at 37° C. for 1 hour before plating on an LB (Luria-Bertani) agar plate containing 30 μg/mL kanamycin and 200 μg/mL ampicillin; the plate was incubated at 37° C. overnight.

A starter culture was grown from a clone overnight in LB medium supplemented with 30 μg/mL kanamycin and 200 μg/mL ampicillin. The starter culture was used to inoculate flasks of terrific broth culture medium with 30 μg/mL kanamycin and 200 μg/mL ampicillin. The culture was incubated at 37° C. (250 rpm), and induced with 500 μM isopropyl-1-thio-β-D-galactopyranoside (IPTG) at OD₆₀₀=1.6−1.8, and expressed at 23° C. (250 rpm) for 14-18 h. Cell pellets were collected, washed three times with PBS, and then resuspended in PBS for incubation with effectors.

These cell suspensions were split into five aliquots, and to each aliquot was added 250 μM Fe(II)SO₄, together with one of the following compounds at a concentration of 10 mM: (1) D-arabinono-1,4-lactone, (2) L-galactono-1,4-lactone, (3) L-gulono-1,4-lactone, (4) L-ascorbic acid, or (5) nothing additional. The cell suspensions were incubated at 30° C. (250 rpm) for 3 hrs. Cell pellets were collected, washed three times with PBS, resuspended in PBS, and then lysed by sonication. The lysate supernatants were collected after centrifugation and the expressed GST-(PPG)₅ was purified using glutathione affinity resin.

GST-(PPG)₅ samples were then incubated with 50 U/(mg protein) of thrombin to cleave GST tags from the (PPG)₅ peptides. Due to the cleavage pattern of thrombin, the resulting peptide was GS(PPG)₅, i.e. Gly-Ser-(Pro-Pro(/Hyp)-Gly)₅ (SEQ ID NO: 39). After 2 hours, the cleaved peptide was separated from GST by applying the proteolysis mix to a 10 kD cutoff spin concentrator and collecting the effluent.

The GST-(PPG)₅ protein with neither P4H nor ALO1 coexpression was previously expressed and purified as a negative control. A positive control experiment was carried out by incubating the purified GST-(PPG)₅ with purified P4H. The preparation of the controls is described in Example 1.

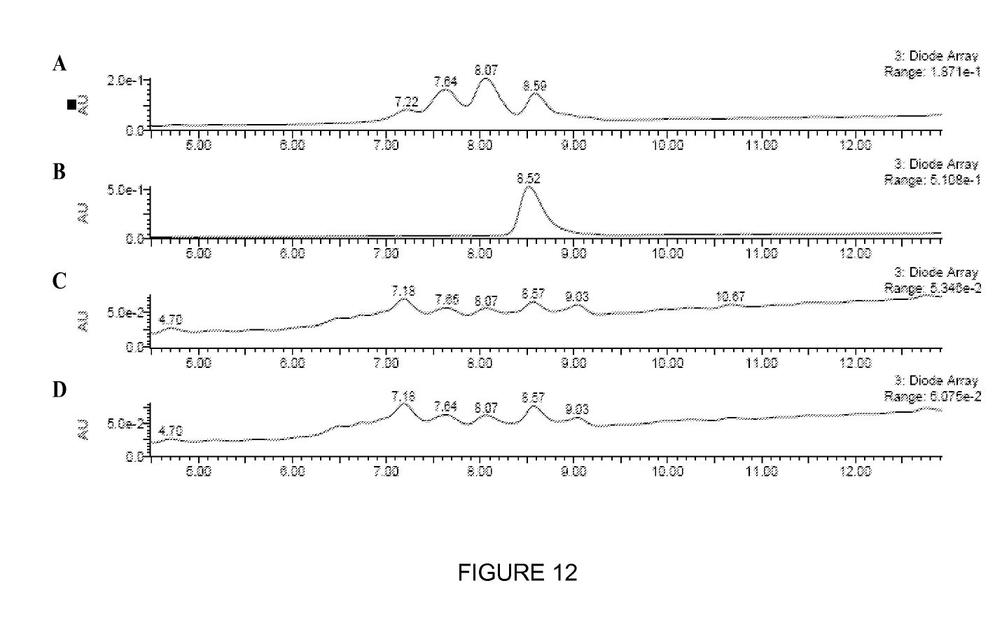

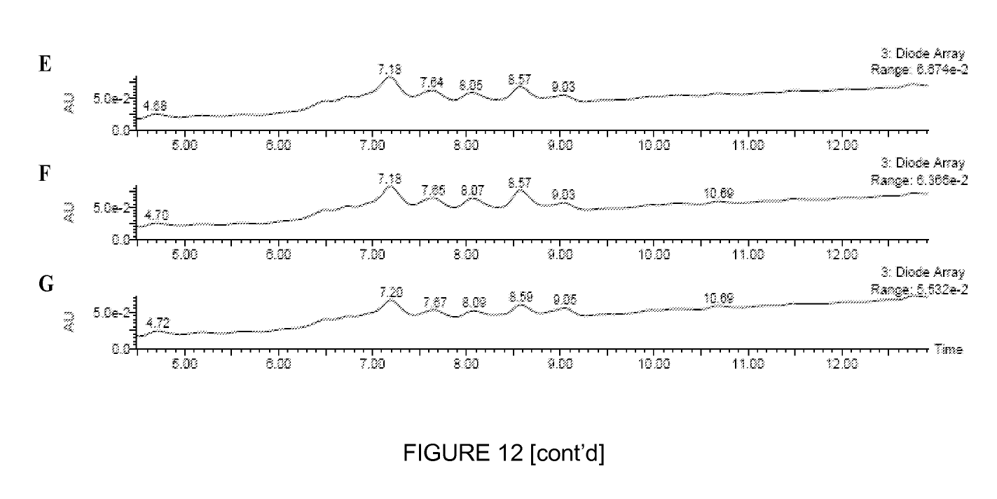

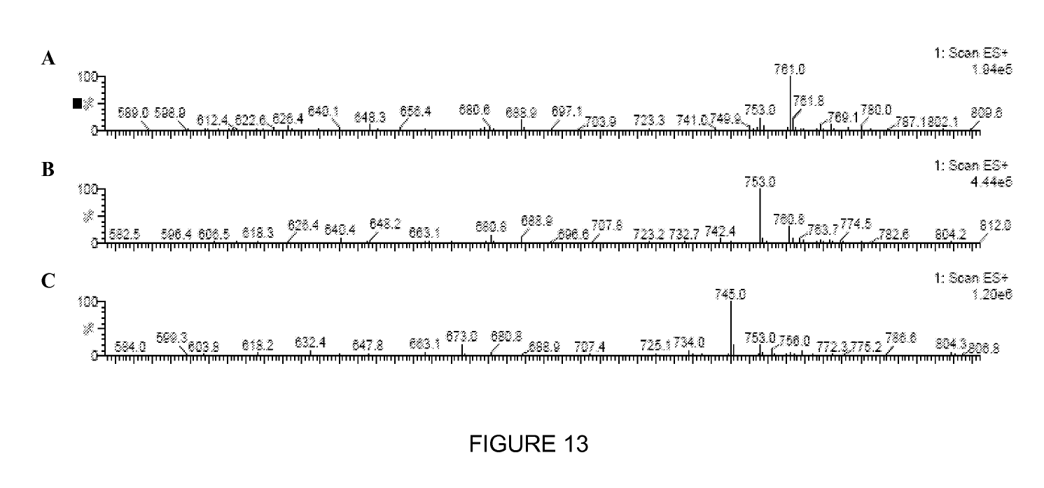

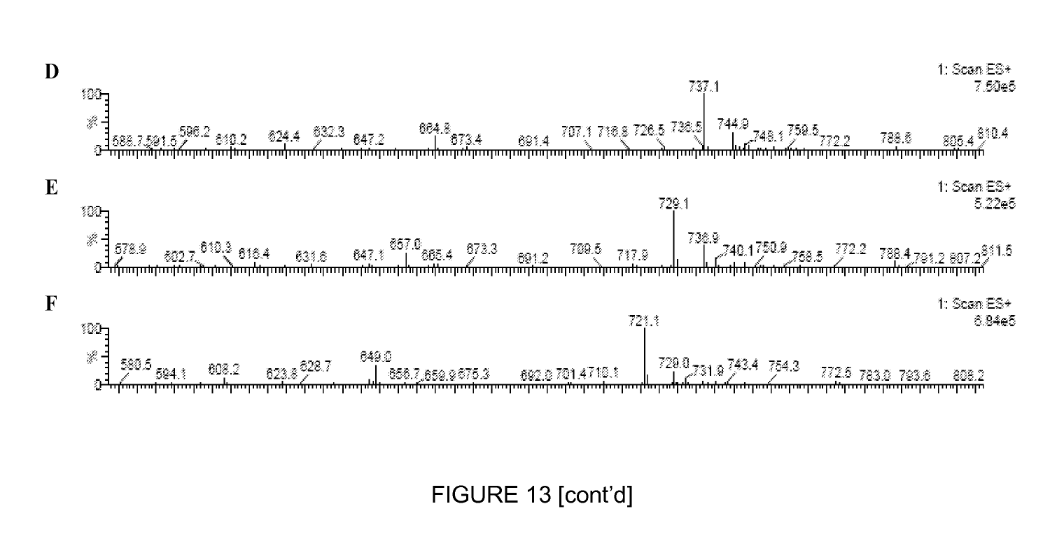

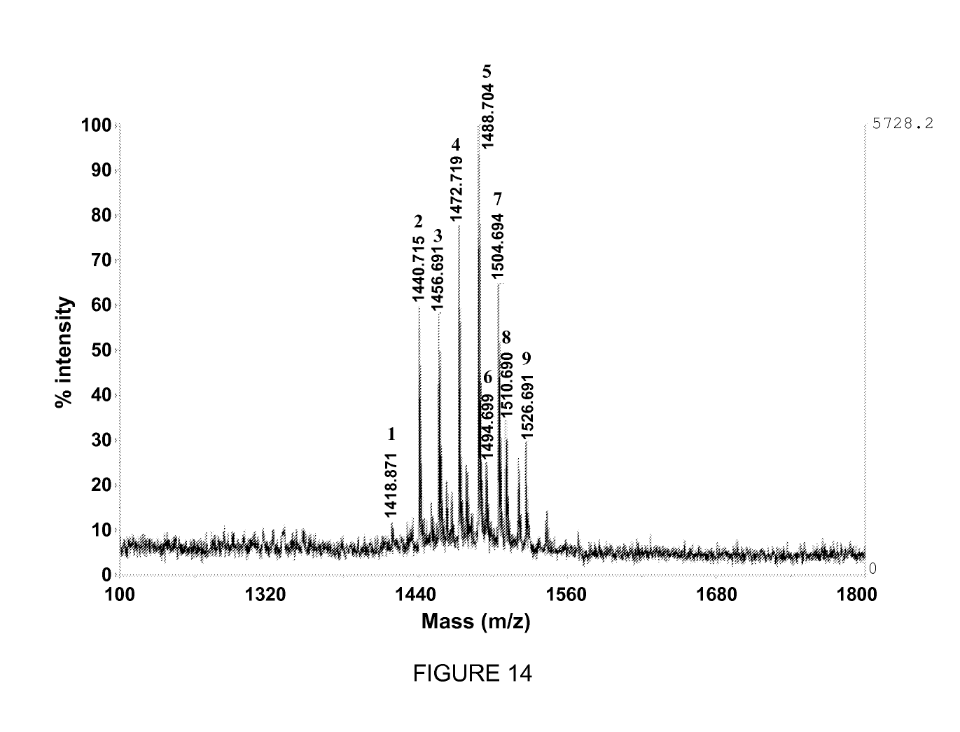

The resulting peptides were analyzed by LC-MS (FIGS. 12 and 13) and MALDI (FIG. 14). All of the samples where hydroxylation was carried out in vivo exhibited similar patterns of hydroxylation (FIGS. 12C-F). Cells expressing P4H and ALO1 incubated with only Fe(II)SO₄ (a presumed negative control) produced a hydroxylation pattern similar to that of all the other experiments (FIG. 12G). The observation of hydroxylation in all of the samples suggests that there is an endogenous source of sugar-1,4-lactone in E. coli that can be used as a substrate for ALO1; thus, unexpectedly, the addition of substrate to the growth media is unnecessary. The LC-MS data shows a clear pattern of hydroxylation having additional m/z=8 in the doubly charged species (FIG. 13), with peaks showing up to 5 hydroxyproline residues (fully hydroxylated) (FIG. 13A). The MALDI result was consistent with the electrospray data obtained by LC-MS (FIG. 14).

### Example 5

### Confirmation of ALO1 Activity in E. coli

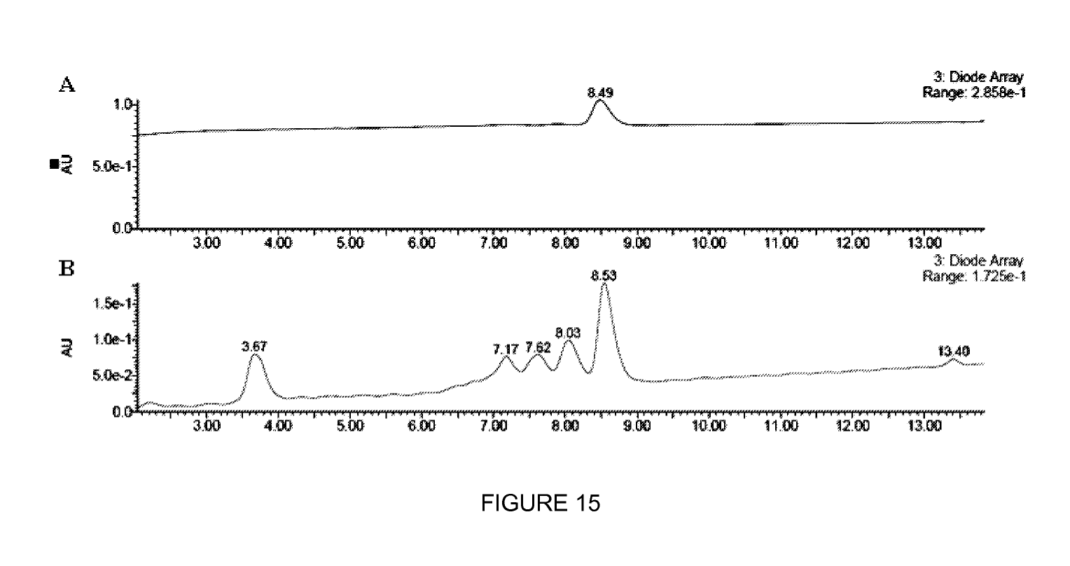

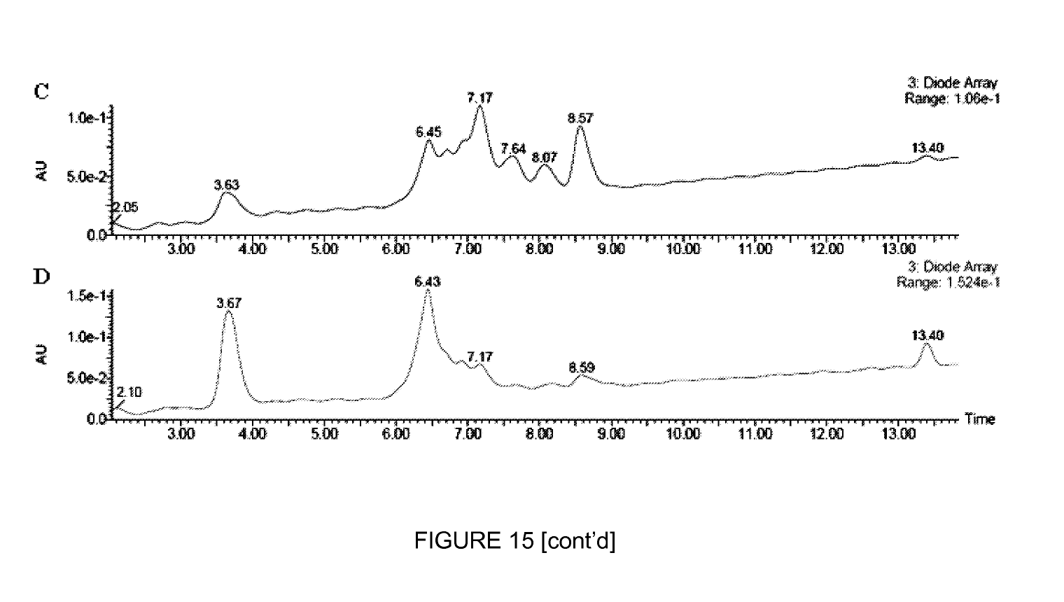

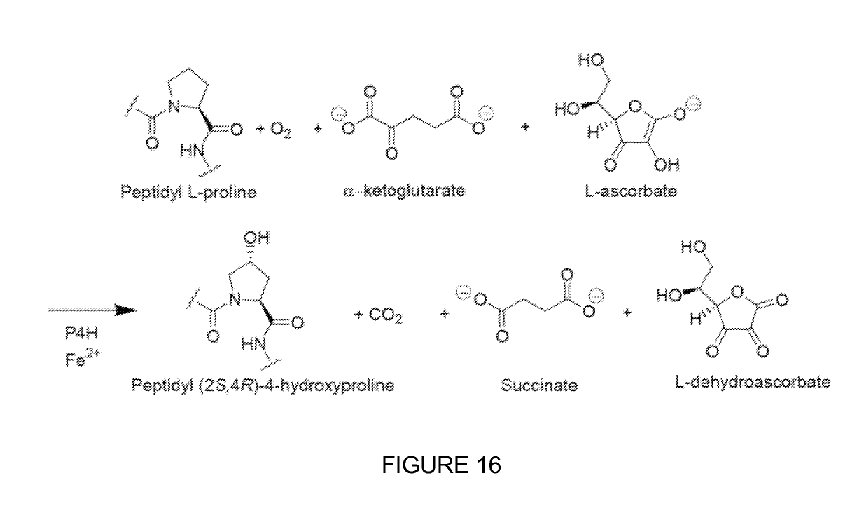

To confirm the hydroxylase activity results shown in Example 4, and to investigate the nutritional requirements for the observed activity, GST-(PPG)₅ was co-expressed with P4H and ALO1 in the Origami 2 strain of E. coli in three different media, along with the negative control of expression in rich media without ALO1 gene. Cultures were grown at 37° C. to OD₆₀₀=0.6 for cultures grown in LB and M9 media, and to OD₆₀₀=1.2 for cultures grown in terrific broth. The temperature was then reduced to 23° C., the cultures were induced with 500 μM IPTG and expressed for 16 hours. After centrifugation, pellets were collected, washed twice with PBS, and then resuspended in PBS with 5 mM EDTA. The cells were then lysed by sonication and the GST-(PPG)₅ was purified using glutathione affinity resin. Effluent from resin purification was concentrated and the peptide GS(PPG)₅ was released from the GST by thrombin cleavage. The released peptides were separated by collecting the flow-through of the cleavage reaction using a 10 kD cutoff filter, diluted to equal concentrations based on pre-cleavage GST-(PPG)₅ protein concentrations, and analyzed by LC-MS (FIG. 15).

The results demonstrated in vivo hydroxylation of the peptide, and confirmed the results described in Example 4. Additionally, the results demonstrated that the precursor of the oxidase substrate was being synthesized by E. coli, since hydroxylation occurred in defined media with no yeast extract.

### Example 6

### Optimization of Shake Flask Culture for High Hydroxylation Levels

An ordinary shake flask culture methodology was optimized for high hydroxylation levels. The concentrations of the purified proteins were determined by UV₂₈₀ₙₘ with a Nanodrop spectrophotometer (Thermo Scientific). In order to remove GST tags from the collagen-like peptides, 4 units thrombin (MP Biomedical) were incubated with 75 μg of protein at room temperature for 2 hours in a final volume of 60 μL in DPBS; 1 mM benzamidine (Sigma-Aldrich) was added to the mixture to stop the cleavage reaction. The samples were analyzed by LC-MS (Waters) equipped with a diode array detector as well as a quadrupole mass spectrometer, with a gradient of 5-95% acetonitrile over 1 hour. Quantitative determination of hydroxylation fraction was achieved by using the areas of extracted ion chromatograms. All relative ionization efficiencies (RIE) were set equal to 1 after verification that the RIE for (Pro-Hyp-Gly)₅ was great than 80% of that for (Pro-Pro-Gly)₅ by comparing the ionization peak areas with UV₂₁₄ₙₘ peak area in chromatograms. The percentage of substrate prolines hydroxylated (H. level %) was calculated as

$$H . level \% = \frac{\text{?} n \times {A}_{n}}{{n}_{ma x} \text{?} {A}_{n}} \times 100 \% , \\ \text{?} \text{indicates text missing or illegible when filed}$$

in which n is the number of hydroxylated substrate prolines (note: only prolines in the Y position of X-Y-Glycine repeats are considered as substrate prolines), nₘₐₓ is the total number of substrate prolines, and Aₙ is the peak area in extracted ion chromatograms of peptide with hydroxylated proline number of n.

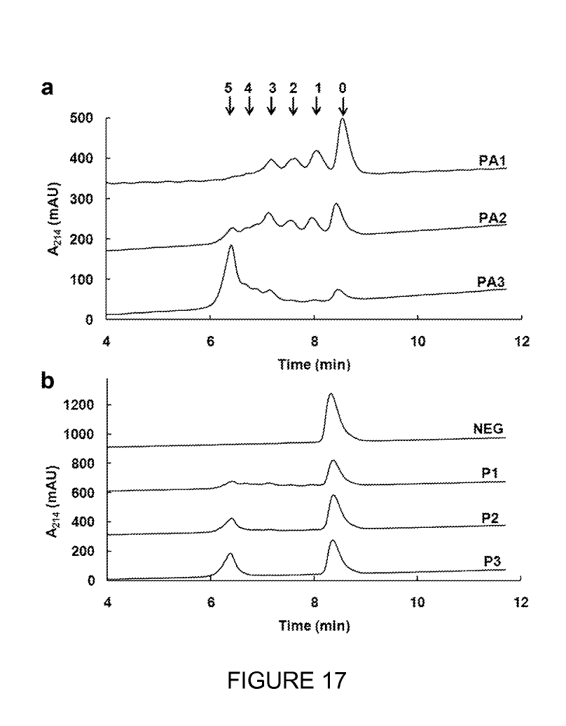

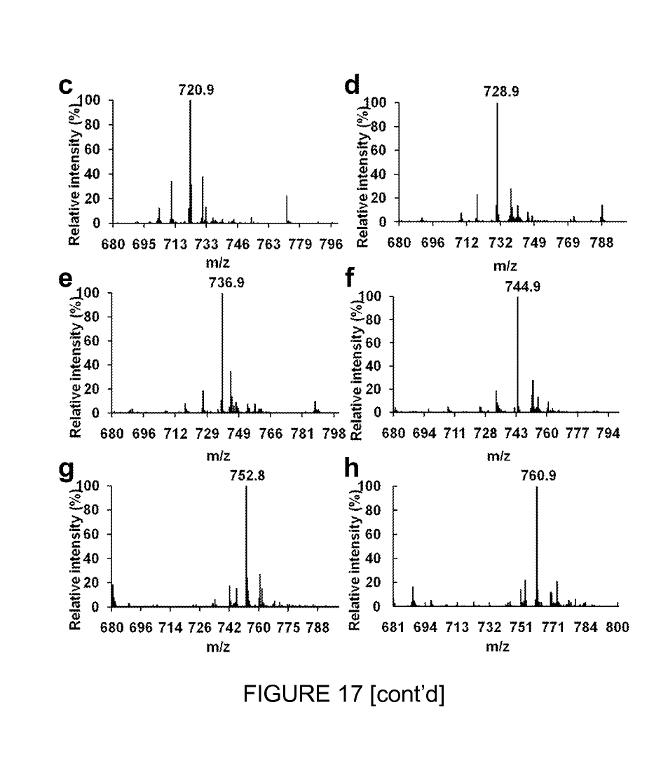

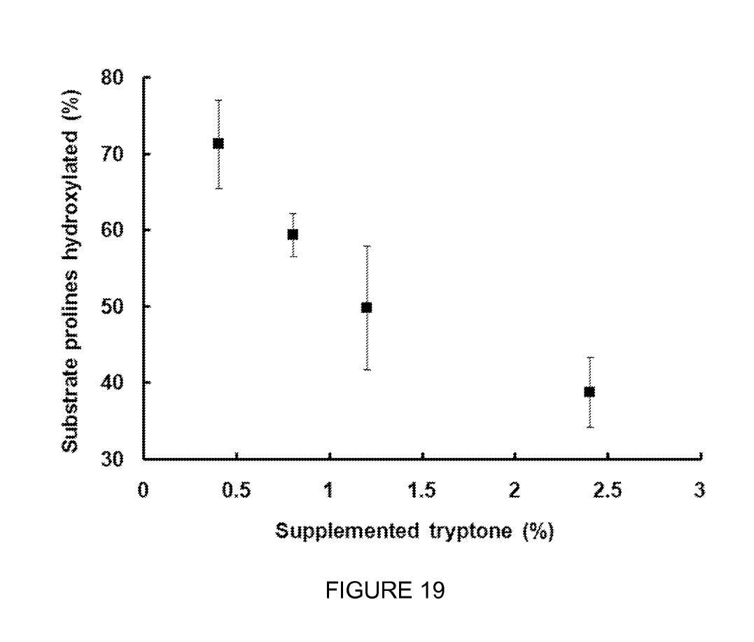

The amount of hydroxylation was strongly dependent on culture media. Minimal media generally yielded higher hydroxylation levels than rich media (FIGS. 17A, 17C-H), with M9 minimal media plus 0.4% tryptone as the carbon source producing the highest hydroxylation levels at 71±6% of Y position prolines. The trend of less-rich media leading to higher hydroxylation levels repeated itself in an experiment where the amount of tryptone was varied while holding other media parameters constant (shown in FIG. 19).

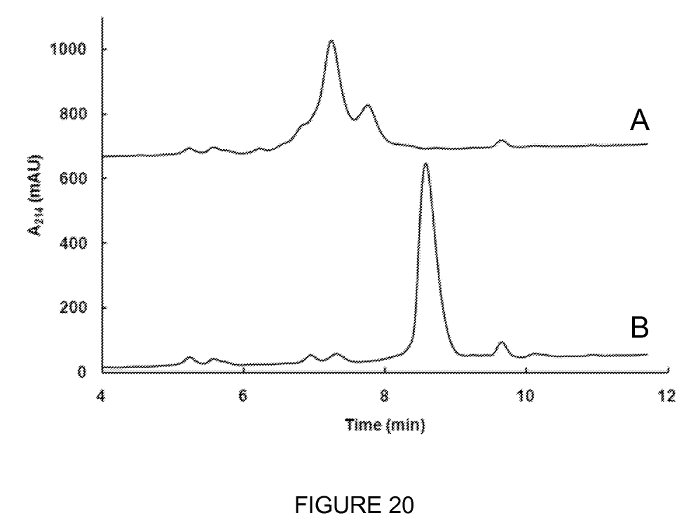

Cultures in which P4H was expressed without ALO1 in rich media showed very low levels of hydroxylation. However, when P4H was expressed without ALO1 in optimized minimal media conditions, hydroxylation was observed in an unexpected “all-or-none” pattern. That is, the expressed (Pro-Pro-Gly)₅ repeats were either nearly fully hydroxylated ((Pro-Hyp-Gly)₅, SEQ ID NO: 58; sequence given is of the peptide after thrombin cleavage), or remained unhydroxylated ((Pro-Pro-Gly)₅, SEQ ID NO: 59; sequence given is of the peptide after thrombin cleavage) (FIG. 17B). The results indicate the existence of a distinct ascorbate-independent mode of processing Pro-Pro-Gly repeats by P4H. No hydroxylation was found in an in vitro experiment where ascorbate was not supplemented (FIG. 20), indicating that this effect is mediated by factors present in E. coli that are not present in vitro.

### Example 7

### Hydroxylation of PPG-Foldon Fusions

In eukaryotes, proper hydroxylation of collagen manifests itself as an increase in the melting temperature. An experiment was conducted to determine whether P4H-mediated hydroxylation observed in the prokaryotic system disclosed herein would function for that purpose. (Pro-Pro-Gly)₅ (SEQ ID NO: 59; sequence given is of the peptide after thrombin cleavage) or (Pro-Pro-Gly) (SEQ ID NO: 60; sequence given is of the peptide after thrombin cleavage) constructs were fused at the C-terminus to the 27-amino-acid T4-phage foldon domain (GSGSGYIPEAPRDGQAYVRKDGEWVLLSTFL) (SEQ ID NO: 61; sequence given is of the peptide after thrombin cleavage) as was done previously for Pro-Pro-Gly synthetic peptide repeats in studies of collagen melting, see Frank et al. (2001, “Stabilization of short collagen-like triple helices by protein engineering,” J. Mol. Biol. 308: 1081-9). The foldon forms an obligate trimer, reducing possible network formation by keeping individual strands attached and aligned at one end, simultaneously raising the melting point of attached collagenous domains.

ALO1 was cloned into a pCOLADuet vector as follows. Genomic DNA from Saccharomyces cerevisiae (strain EBY100) was extracted using Gentra Puregene Yeast/Bact. Kit (Qiagen) according to the manufacturer's instructions. cDNA encoding ALO1 was amplified from yeast genomic DNA using primers described by Lee et al. (1999, Appl Environ Microbiol 65: 4685-7), which introduced a BamHI site at the 3′ end of the gene and an NdeI site at the 5′ end. The PCR product resulting from this amplification was digested with NdeI and BamHI (all restriction enzymes from New England Biolabs) and then ligated into a pET19b vector using T4 ligase, resulting in the plasmid named “pSD.ET19b.ALO1”. In order to generate an ALO1 (the gene encoding ALO) insert with restriction sites appropriate for insertion into the co-expression vector pCOLADuet-1 (Novagen), PCR was performed on plasmid “pSD.ET19b.ALO1” using primers 5′-CCCGAAAGGAAGCTCGAGTTGGCTGCTG-3′ (SEQ ID NO: 35) and 5′-CAGCAGCCAACTCGAGCTTCCTTTCGGG-3′ (SEQ ID NO: 36). The PCR produced a linear fragment with a XhoI site on the 3′ side of the stop codon of ALO1 gene while retaining the NdeI site on its 5′ side. The resulting fragment was then digested with NdeI and XhoI restriction enzymes, and ligated into the 2^{nd} multiple cloning site (MCS) of pCOLADuet-1 vector, resulting in a plasmid named “pSD.COLADuet-1.0.ALO1” (see FIG. 18 for vector map of similar plasmid pSD.COLADuet-1.GST-(PPG)₅.ALO1).

GST-(PPG)₅ was cloned into pCOLADuet expression vectors as follows. An oligonucleotide encoding (Pro-Pro-Gly)₅ (SEQ ID NO: 25) with a BamHI restriction site at the 5′ end and an XhoI site at the 3′ end was amplified by PCR using primers GCTAGGATCCCCGCCGGGTC (SEQ ID NO: 26) and CTAGCTCGAGTTAACCAGGC (SEQ ID NO: 27) to obtain a specific double-stranded DNA. The PCR product was digested with BamHI and XhoI, and ligated into vector pGEX4T-1 (GE healthcare) in order to create the fusion of (Pro-Pro-Gly)₅ to glutathione S-transferase (GST) with an intervening thrombin protease cleavage site (“pSD.GEX4T-1.GST-(PPG)₅”). In order to introduce appropriate restriction sites to ligate GST-(Pro-Pro-Gly)₅ into the 1^{st} MCS of pCOLADuet-1 vector, PCR was carried out on the plasmid “pSD.GEX4T-1.GST-(PPG)₅”, using primers 5′-CAGCTACCAT GGGTTCCCCT ATACTAGGTT ATTGGAAAAT TAAGGGCC-3′ (SEQ ID NO: 28) and 5′-CCTGACGGGC TTGTCTGCTC CC-3′ (SEQ ID NO: 29) that introduced a NcoI site on the 5′ side of the translation initiation codon of GST-(Pro-Pro-Gly)₅ and a NotI site after the 3′ side of the stop codon. The PCR fragment was digested with NcoI and NotI, and ligated into the 1^{st} MCS of both the empty pCOLADuet-1 vector and the plasmid “pSD.COLADuet-1.0.ALO1”, which created plasmids “pSD.COLADuet-1.GST-(PPG)₅.0” and “pSD.COLADuet-1.GST-(PPG)₅.ALO1” (see FIG. 18 for plasmid map), respectively.

The DNA encoding (Pro-Pro-Gly)₁₀-foldon (SEQ ID NO: 62; sequence given is of the peptide after thrombin cleavage):

(SEQ ID NO: 63)
5′-GGATCCCCTGGTGCCGCGTGGCAGCGGTCCGCCGGGCCCGCCGGGC
CCGCCGGGTCCGCCGGGACCTCCGGGTCCTCCTGGCCCTCCTGGTCCGC
CGGGACCCCCGGGTCCGCCGGGCAGCGGTTATATTCCGGAAGCACCGCG
TGATGGTCAGGCATACGTGCGTAAAGATGGCGAATGGGTTCTGCTGTCT
ACCTTTCTGTAAGCGGCCGC-3′

was synthesized (Genscript), and PCR-amplified from the supplied vector using primers 5′-GTGCCGCGTGGATCCGGTCCGCC-3′ (SEQ ID NO: 64) and 5′-ACGATGCGGCCGCTTACAGAAAGGTAGACAG-3′ (SEQ ID NO: 65). The PCR product and the plasmid “pSD.COLADuet-1.GST-(PPG)₅.0” were both digested with BamHI and NotI, and then ligated. This resulted in plasmid “pSD.COLADuet-1.GST-(PPG)₁₀-foldon.0”.

The DNA encoding GST-(Pro-Pro-Gly)₁₀-foldon (SEQ ID NO: 66) was then isolated from the plasmid by NcoI and NotI digestion and gel extraction, and then ligated into the 1^{st} MCS of plasmid “pSD.COLADuet-1.0.ALO1”, resulting in plasmid “pSD.COLADuet-1. GST-(PPG)₁₀-foldon.ALO1”.

Stop codons were introduced just after (Pro-Pro-Gly)₁₀ (SEQ ID NO: 67; sequence given is of the peptide after thrombin cleavage) in both plasmids “pSD.COLADuet-1.GST-(PPG)₁₀-foldon.0” and “pSD.COLADuet-1.GST-(PPG)₁₀-foldon.ALO1” by site-directed mutagenesis per the Stratagene Quickchange protocol using primers 5′-GACCCCCGGGTCCGCCGTGAGCGGTTATATTC-3′ (SEQ ID NO: 68) and 5′-GAATATAACCGCTCACGGCGGACCCGGGGGTC-3′ (SEQ ID NO: 69). This resulted in plasmids “pSD.COLADuet-1.GST-(PPG)₁₀.0” and “pSD.COLADuet-1.GST-(PPG)₁₀.ALO1”. Similarly, by introducing stop codons 3′ to (Pro-Pro-Gly)₇ (SEQ ID NO: 60; sequence given is of the peptide after thrombin cleavage) using primers 5′-CTGGCCCTCCTTGACGGGACCCCCGGGTCCG-3′ (SEQ ID NO: 70) and 5′-CGGACCCGGGGGTCCCGTCAAGGAGGGCCAG-3′ (SEQ ID NO: 71), plasmids “pSD.COLADuet-1.GST-(PPG)₇.0” and “pSD.COLADuet-1.GST-(PPG)₇.ALO1” were obtained.

In order to create plasmids encoding GST-foldon, GST-(Pro-Pro-Gly)₅-foldon (SEQ ID NO: 72) and GST-(Pro-Pro-Gly)₇-foldon (SEQ ID NO: 73), deletion mutagenesis was performed on the plasmids “pSD.COLADuet-1.GST-(PPG)₁₀-foldon.ALO1” and “pSD.COLADuet-1.GST-(PPG)₁₀-foldon.0” according to the strategy described by Liu et al. (Liu & Naismith, 2008, BMC Biotechnol 8: 91). Briefly, primers were designed to contain “non-overlapping” sequences (primer-plasmid complementary) at their 3′ end and “primer-primer complementary” sequences at the 5′ end. The melting temperature of non-overlapping sequences (T_{m no}) was 5 to 10° C. higher than the melting temperature of the primer-primer complementary sequences (T_{m pp}). Twelve cycles of PCR were performed of the following treatment: 95° C. for 1 minute, T_{m no} −5° C. for 1 minute, and 72° C. for 10 minutes. The PCR cycles were followed by T_{m pp} −5° C. for 1 minute and 72° C. for 30 minutes. The PCR mixture was incubated with Dpnl, and then transformed the PCR product mixture into NovaBlue competent cells, followed by screening the colonies to check the DNA sequence. Similarly, primers 5′-GGCAGCGGTTATATTCCGGAAGCACCG-3′ (SEQ ID NO: 74) and 5′-ATATAACCGCTGCCGGATCCACGCGGAACCAGATCC-3′ (SEQ ID NO: 75) were used to generate plasmid “pSD.COLADuet-1.GST-foldon.0”. Using primers 5′-CGCGTGGATCCGGTCCTCCTGGCCCTCCTGGTC-3′ (SEQ ID NO: 76) and 5′-GGATCCACGCGGAACCAGATCCGATTTTG-3′ (SEQ ID NO: 77), plasmids “pSD.COLADuet-1.GST-(PPG)₅-foldon.ALO1” and “pSD.COLADuet-1.GST-(PPG)₅-foldon.0” were generated, and PCR with primers 5′-GGCAGCGGTTATATTCCGGAAGCACCG-3′ (SEQ ID NO: 78) and 5′-ATATAACCGCTGCCAGGAGGGCCAGGAGGACCC-3′ (SEQ ID NO: 79) resulted in plasmids “pSD.COLADuet-1.GST-(PPG)₇-foldon.ALO1” and “pSD.COLADuet-1.GST-(PPG)₇-foldon.0”.

The proteins containing foldon were cleaved by Thrombin CleanCleave™ Kit (Sigma), and the cleaved products were separated from GST tag and uncleaved products by applying the mixture to glutathione affinity resin and collecting the flow through. The peptides were then concentrated using a 3 kDa cut off Amicon protein concentrator (Millipore), heated at 95° C. for 5 minutes, and applied to a 0.2 μm spin filter microcon (Millipore) to remove possible residual protein impurities. The purity of the products was checked by SDS-PAGE and analytical HPLC (Waters), and the final peptide concentration was determined by measuring UV₂₆₀ₙₘ in Nanodrop spectrophotometer and analytical HPLC.

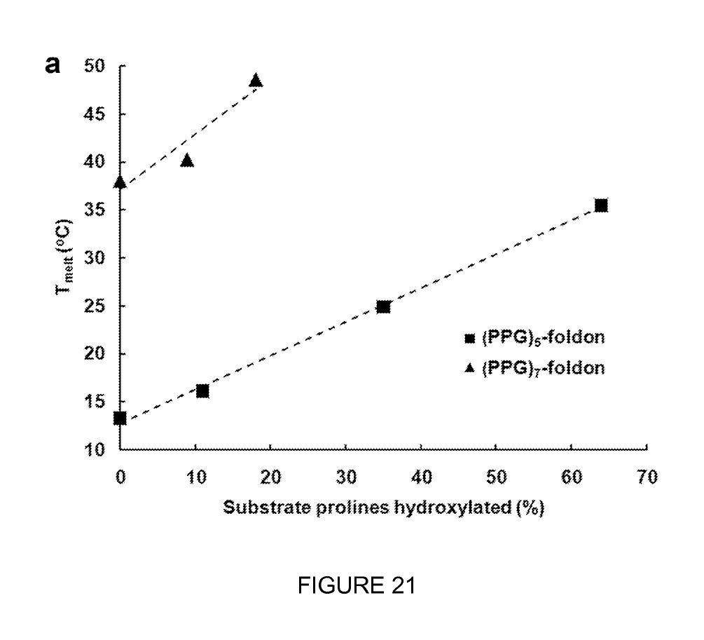

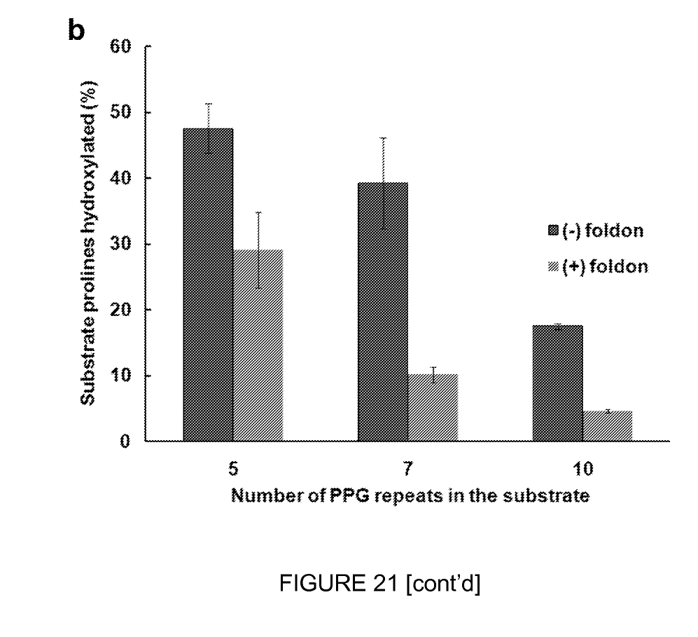

(Pro-Pro-Gly)₅-foldon (SEQ ID NO: 80; sequence given is of the peptide after thrombin cleavage) and (Pro-Pro-Gly)₇-foldon (SEQ ID NO: 81; sequence given is of the peptide after thrombin cleavage) constructs were hydroxylated to different extents by varying culture conditions and measuring the resulting melting temperatures by circular dichroism using a previously described method (see Boudko et al., 2002, “Nucleation and propagation of the collagen triple helix in single-chain and trimerized peptides: transition from third to first order kinetics,” J. Mol. Biol. 317: 459-70). The spectra of the peptides (55 μM in DPBS buffer) were acquired in Jasco J-815 CD spectrometer with a 1 mm path length quartz cell. The ellipticity at 210 nm was then monitored from −10° C. to 80° C. as the temperature was increased at a rate of 1° C. per minute. The thermal transition curve was defined as three phases: pre-melt, melting, and post-melt, which were each linearly fit into a line. The value of Tₘₑₗₜ was determined as the temperature at the midpoint of the intersections. Melting points were found to increase with hydroxylation level for both constructs (FIG. 21A).

The system was then used to investigate how P4H interacts with collagen as it folds. A series of (Pro-Pro-Gly)ₙ constructs were produced, wherein n=5, 7, and 10, that were either expressed as fusions to a foldon domain or left unfused. These constructs were chosen for having melting points ranging from well above culture conditions for unhydroxylated (Pro-Pro-Gly)₁₀-foldon (SEQ ID NO: 62; sequence given is of the peptide after thrombin cleavage) (63±2° C.) to not capable of forming trimers even when fully hydroxylated ((Pro-Hyp-Gly)₅, SEQ ID NO: 58). This series of collagenous materials was produced in bacteria coexpressing both P4H and ALO1 in M9 minimal media plus 0.4% tryptone and 0.4% glycerol. Foldon-fused Pro-Pro-Gly repeats consistently exhibited lower extents of hydroxylation than their unfused counterparts (FIG. 21B). Also, the longer Pro-Pro-Gly repeats were disproportionately less hydroxylated (FIG. 21B). These data indicate that P4H-mediated hydroxylation is dependent on the folded state of the collagenous material in the E. coli expression system.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

What is claimed is:

1. A bacterial cell capable of expressing recombinant proteins comprising:
a) one or more nucleic acids encoding a sugar-1,4-lactone oxidase or a sugar-1,4-lactone dehydrogenase; and
b) one or more nucleic acids encoding an ascorbate-dependent biosynthetic enzyme.

2. The bacterial cell of claim 1, wherein the one or more nucleic acids encoding the sugar-1,4-lactone oxidase or sugar-1,4-lactone dehydrogenase comprise a first expression vector, and the one or more nucleic acids encoding the ascorbate-dependent biosynthetic enzyme comprise a second expression vector.

3. The bacterial cell of claim 1, wherein the nucleic acids encoding the sugar-1,4-lactone oxidase or sugar-1,4-lactone dehydrogenase and the ascorbate-dependent biosynthetic enzyme comprise a single expression vector.

4. The bacterial cell of claim 1,
wherein the sugar-1,4-lactone oxidase is D-arabinono-1,4-lactone oxidase, L-gulono-1,4-lactone oxidase, or D-glucono-1,4-lactone oxidase; and
wherein the sugar-1,4-lactone dehydrogenase is D-arabinose dehydrogenase, L-gulono-1,4-lactone dehydrogenase, L-gulono-γ-lactone dehydrogenase, D-glucose dehydrogenase, L-galactono-1,4-lactone dehydrogenase, L-galactono-γ-lactone dehydrogenase, L-sorbosone dehydrogenase, or 2-ketogluconate dehydrogenase.

5. (canceled)

6. The bacterial cell of claim 1, wherein the ascorbate-dependent biosynthetic enzyme is a hydroxylase, wherein the hydroxylase is prolyl-4-hydroxylase, prolyl-3-hydroxylase, lysyl-5-hydroxylase, HIF prolyl hydroxylase, aspartyl beta-hydroxylase, asparaginyl beta-hydroxylase, or HIF asparaginyl hydroxylase.

7-9. (canceled)

10. The bacterial cell of claim 6, further comprising one or more nucleic acids encoding a peptide or a protein to be hydroxylated.

11. The bacterial cell of claim 10, wherein the one or more nucleic acids encoding the sugar-1,4-lactone oxidase or sugar-1,4-lactone dehydrogenase comprise the first expression vector; the one or more nucleic acids encoding the hydroxylase comprise the second expression vector; and the one or more nucleic acids encoding the peptide or the protein to be hydroxylated comprise a third expression vector.

12. The bacterial cell of claim 10, wherein the one or more nucleic acids encoding the sugar-1,4-lactone oxidase or sugar-1,4-lactone dehydrogenase and the peptide or the protein to be hydroxylated comprise a first expression vector, and the one or more nucleic acids encoding the hydroxylase comprise a second expression vector.

13. The bacterial cell of claim 10, wherein the one or more nucleic acids encoding the hydroxylase and the peptide or protein to be hydroxylated comprise a first expression vector, and the one or more nucleic acids encoding the sugar-1,4-lactone oxidase or sugar-1,4-lactone dehydrogenase comprise a second expression vector.

14. The bacterial cell of claim 10, wherein the one or more nucleic acids encoding the hydroxylase and the sugar-1,4-lactone oxidase or sugar-1,4-lactone dehydrogenase comprise a first expression vector, and the one or more nucleic acids encoding the peptide or the protein to be hydroxylated comprise a second expression vector.

15. The bacterial cell of claim 10, wherein the nucleic acids encoding the sugar-1,4-lactone oxidase or sugar-1,4-lactone dehydrogenase, the hydroxylase, and the peptide or protein to be hydroxylated comprise a single expression vector.

16. The bacterial cell of claim 10,
wherein the sugar-1,4-lactone oxidase is D-arabinono-1,4-lactone oxidase, L-gulono-1,4-lactone oxidase, or D-glucono-1,4-lactone oxidase; and
wherein the sugar-1,4-lactone dehydrogenase is D-arabinose dehydrogenase, L-gulono-1,4-lactone dehydrogenase, L-gulono-γ-lactone dehydrogenase, D-glucose dehydrogenase, L-galactono-1,4-lactone dehydrogenase, L-galactono-γ-lactone dehydrogenase, L-sorbosone dehydrogenase, or 2-ketogluconate dehydrogenase.

17. (canceled)

18. The bacterial cell of claim 10, wherein the ascorbate-dependent biosynthetic enzyme is prolyl-4-hydroxylase, prolyl-3-hydroxylase, lysyl-5-hydroxylase, HIF prolyl hydroxylase, aspartyl beta-hydroxylase, asparaginyl beta-hydroxylase, or HIF asparaginyl hydroxylase.

19-20. (canceled)

21. The bacterial cell of claim 10, wherein the peptide or the protein to be hydroxylated is collagen.

22. The bacterial cell of claim 1 that is an Escherichia coli cell.

23. A method of making a post-translationally hydroxylated recombinant protein comprising expressing in the bacterial cell according to claim 1 one or more nucleic acids encoding a peptide or a protein to be hydroxylated, wherein the ascorbate-dependent biosynthetic enzyme is a hydroxylase.

24. The method of claim 23, wherein the bacterial cell comprises:
a first expression vector comprising the one or more nucleic acids encoding the sugar-1,4-lactone oxidase or sugar-1,4-lactone dehydrogenase;
a second expression vector comprising the one or more nucleic acids encoding the hydroxylase; and
a third expression vector comprising the one or more nucleic acids encoding the peptide or the protein to be hydroxylated.

25. The method of claim 23, wherein the bacterial cell comprises:
a first expression vector comprising the one or more nucleic acids encoding the sugar-1,4-lactone oxidase or sugar-1,4-lactone dehydrogenase and the peptide or the protein to be hydroxylated; and
the second expression vector comprising the one or more nucleic acids encoding the hydroxylase.

26. The method of claim 23, wherein the bacterial cell comprises:
a first expression vector comprising the one or more nucleic acids encoding the hydroxylase and the peptide or the protein to be hydroxylated; and
a second expression vector comprising the one or more nucleic acids encoding the sugar-1,4-lactone oxidase or sugar-1,4-lactone dehydrogenase.

27. The method of claim 23, wherein the bacterial cell comprises:
a first expression vector comprising the one or more nucleic acids encoding the hydroxylase and the sugar-1,4-lactone oxidase or sugar-1,4-lactone dehydrogenase; and
a second expression vector comprising the one or more nucleic acids encoding the peptide or the protein to be hydroxylated.

28. The method of claim 23, wherein the bacterial cell comprises an expression vector comprising the nucleic acids encoding the sugar-1,4-lactone oxidase or sugar-1,4-lactone dehydrogenase, the hydroxylase, and the peptide or the protein to be hydroxylated.

29. The method of claim 23,
wherein the sugar-1,4-lactone oxidase is D-arabinono-1,4-lactone oxidase, L-gulono-1,4-lactone oxidase, or D-glucono-1,4-lactone oxidase; and
wherein the sugar-1,4-lactone dehydrogenase is D-arabinose dehydrogenase, L-gulono-1,4-lactone dehydrogenase, L-gulono-γ-lactone dehydrogenase, D-glucose dehydrogenase, L-galactono-1,4-lactone dehydrogenase, L-galactono-γ-lactone dehydrogenase, L-sorbosone dehydrogenase, or 2-ketogluconate dehydrogenase.

30. (canceled)

31. The method of claim 23, wherein the hydroxylase is prolyl-4-hydroxylase, prolyl-3-hydroxylase, lysyl-5-hydroxylase, HIF prolyl hydroxylase, aspartyl beta-hydroxylase, asparaginyl beta-hydroxylase, or HIF asparaginyl hydroxylase.

32-33. (canceled)

34. The method of claim 23, wherein the peptide or the protein to be hydroxylated is collagen.

35. The method of claim 23, wherein the bacterial host cell is Escherichia coli.

36. A post-translationally hydroxylated recombinant collagen molecule produced by a method comprising the step of co-expressing in a bacterial cell one or more nucleic acids encoding collagen, one or more nucleic acids encoding a sugar-1,4-lactone oxidase or a sugar-1,4-lactone dehydrogenase, and one or more nucleic acids encoding an ascorbate-dependent biosynthetic enzyme, wherein the ascorbate-dependent biosynthetic enzyme is prolyl-4-hydroxylase, prolyl-3-hydroxylase, or lysyl-5-hydroxylase.

37. The collagen molecule of claim 36, wherein the one or more nucleic acids encoding the sugar-1,4-lactone oxidase or sugar-1,4-lactone dehydrogenase comprise a first expression vector; the one or more nucleic acids encoding the ascorbate-dependent biosynthetic enzyme comprise a second expression vector; and the one or more nucleic acids encoding collagen comprise a third expression vector.

38. The collagen molecule of claim 36, wherein the one or more nucleic acids encoding the sugar-1,4-lactone oxidase or sugar-1,4-lactone dehydrogenase and collagen comprise a first expression vector, and the one or more nucleic acids encoding the ascorbate-dependent biosynthetic enzyme comprise the second expression vector.

39. The collagen molecule of claim 36, wherein the one or more nucleic acids encoding the ascorbate-dependent biosynthetic enzyme and collagen comprise a first expression vector, and the one or more nucleic acids encoding the sugar-1,4-lactone oxidase or sugar-1,4-lactone dehydrogenase comprise a second expression vector.

40. The collagen molecule of claim 36, wherein the one or more nucleic acids encoding the ascorbate-dependent biosynthetic enzyme and the sugar-1,4-lactone oxidase or sugar-1,4-lactone dehydrogenase comprise a first expression vector, and the one or more nucleic acids encoding collagen comprise a second expression vector.

41. The collagen molecule of claim 36, wherein the nucleic acids encoding the sugar-1,4-lactone oxidase or sugar-1,4-lactone dehydrogenase, the ascorbate-dependent biosynthetic enzyme, and collagen comprise a single expression vector.

42. The collagen molecule of claim 36,
wherein the sugar-1,4-lactone oxidase is D-arabinono-1,4-lactone oxidase, L-gulono-1,4-lactone oxidase, or D-glucono-1,4-lactone oxidase; and
wherein the sugar-1,4-lactone dehydrogenase is D-arabinose dehydrogenase, L-gulono-1,4-lactone dehydrogenase, L-gulono-γ-lactone dehydrogenase, D-glucose dehydrogenase, L-galactono-1,4-lactone dehydrogenase, L-galactono-γ-lactone dehydrogenase, L-sorbosone dehydrogenase, or 2-ketogluconate dehydrogenase.

43-44. (canceled)

45. The collagen molecule of claim 36, wherein the bacterial host cell is Escherichia coli.

46. A Gram-negative bacterial cell capable of expressing recombinant proteins comprising one or more nucleic acids encoding an ascorbate-dependent biosynthetic enzyme or an ascorbate-analog-dependent biosynthetic enzyme,
wherein the enzyme is expressed in the periplasmic space of the bacterial cell; and
wherein ascorbate or an ascorbate analog is supplied exogeneously.

47. The bacterial cell of claim 46, wherein the ascorbate-dependent biosynthetic enzyme is a hydroxylase, wherein the hydroxylase is prolyl-4-hydroxylase, prolyl-3-hydroxylase, lysyl-5-hydroxylase, HIF prolyl hydroxylase, aspartyl beta-hydroxylase, asparaginyl beta-hydroxylase, or HIF asparaginyl hydroxylase.

48. The bacterial cell of claim 47, further comprising one or more nucleic acids encoding a peptide or a protein to be hydroxylated, wherein the peptide or the protein to be hydroxylated is expressed in the periplasmic space of the bacterial cell.

49. The bacterial cell of claim 48, wherein the one or more nucleic acids encoding the hydroxylase comprise a first expression vector, and the one or more nucleic acids encoding the peptide or protein to be hydroxylated comprise a second expression vector.

50. The bacterial cell of claim 48, wherein the nucleic acids encoding the hydroxylase and the peptide or protein to be hydroxylated comprise a single expression vector.

51-53. (canceled)

54. The bacterial cell of claim 48, wherein the peptide or protein to be hydroxylated is collagen.

55. The bacterial cell of claim 46 that is an Escherichia coli cell.

56. (canceled)

57. A method of making a post-translationally hydroxylated recombinant protein comprising expressing in the Gram-negative bacterial cell of claim 48 one or more nucleic acids encoding a peptide or protein to be hydroxylated.

58. The method of claim 57, wherein the one or more nucleic acids encoding the hydroxylase comprise a first expression vector, and the nucleic acid encoding the protein comprises a second expression vector.

59. The method of claim 57, wherein the nucleic acids encoding the hydroxylase and the protein comprise a single expression vector.

60. The method of claim 55, wherein the hydroxylase is prolyl-4-hydroxylase, prolyl-3-hydroxylase, lysyl-5-hydroxylase.

61-62. (canceled)

63. The method of claim 57, wherein the protein is collagen.

64. The method of claim 57, wherein the bacterial host cell is Escherichia coli.

65. A post-translationally hydroxylated recombinant collagen molecule produced in a Gram-negative bacterial host cell co-expressing nucleic acids encoding said collagen molecule and one or more nucleic acids encoding an ascorbate-dependent biosynthetic enzyme, wherein the ascorbate-dependent biosynthetic enzyme is prolyl-4-hydroxylase, prolyl-3-hydroxylase, or lysyl-5-hydroxylase.

66. The collagen molecule of claim 65, wherein the one or more nucleic acids encoding the ascorbate-dependent biosynthetic enzyme comprise a first expression vector, and the nucleic acids encoding the collagen molecule comprises a second expression vector.

67. The collagen molecule of claim 65, wherein the nucleic acids encoding the ascorbate-dependent biosynthetic enzyme and the collagen molecule comprise a single expression vector.

68. (canceled)

69. The collagen molecule of claim 65, wherein the bacterial host cell is Escherichia coli.

70. The bacterial cell of claim 1 or 46, wherein one or more of the nucleic acids encoding the sugar-1,4-lactone oxidase or sugar-1,4-lactone dehydrogenase and the ascorbate-dependent biosynthetic enzyme are incorporated into the bacterial chromosome.

71. The bacterial cell of claim 46, wherein one or more of the nucleic acids encoding the sugar-1,4-lactone oxidase or sugar-1,4-lactone dehydrogenase and the ascorbate-dependent biosynthetic enzyme are incorporated into the bacterial chromosome.

72. The bacterial cell of claim 10 or 48, wherein one or more of the nucleic acids encoding the sugar-1,4-lactone oxidase or sugar-1,4-lactone dehydrogenase, the hydroxylase, and the peptide or protein to be hydroxylated are incorporated into the bacterial chromosome.

73. The bacterial cell of claim 48, wherein one or more of the nucleic acids encoding the sugar-1,4-lactone oxidase or sugar-1,4-lactone dehydrogenase, the hydroxylase, and the peptide or protein to be hydroxylated are incorporated into the bacterial chromosome.

74. The collagen molecule of claim 36, wherein one or more of the nucleic acids encoding the sugar-1,4-lactone oxidase or sugar-1,4-lactone dehydrogenase, the hydroxylase, and the collagen molecule are incorporated into the bacterial chromosome.

75. The method of claim 23, wherein one or more of the nucleic acids encoding the sugar-1,4-lactone oxidase or sugar-1,4-lactone dehydrogenase, the hydroxylase, and the peptide or protein to be hydroxylated are incorporated into the bacterial chromosome.

76. The method of claim 57, wherein one or more of the nucleic acids encoding the sugar-1,4-lactone oxidase or sugar-1,4-lactone dehydrogenase, the hydroxylase, and the peptide or protein to be hydroxylated are incorporated into the bacterial chromosome.

77. A bacterial cell according to claim 1 capable of producing a hydroxylated recombinant protein comprising a collagenous domain that is sufficiently hydroxylated to form a triple-helical structure.

78. A bacterial cell according to claim 10 that produces a hydroxylated recombinant protein comprising a collagenous domain that is sufficiently hydroxylated to form a triple-helical structure.

79. The method of claim 23, wherein the post-translationally hydroxylated recombinant protein comprises a collagenous domain that is sufficiently hydroxylated to form a triple-helical structure.

80. The post-translationally hydroxylated recombinant collagen molecule of claim 36, wherein the collagenous domain is sufficiently hydroxylated to form a triple-helical structure.

81. The Gram-negative bacterial cell of claim 46, that is capable of producing a hydroxylated recombinant protein comprising a collagenous domain that is sufficiently hydroxylated to form a triple-helical structure.

82. The method of claim 57 wherein the post-translationally hydroxylated recombinant protein comprises a collagenous domain that is sufficiently hydroxylated to form a triple-helical structure.

83. The post-translationally hydroxylated recombinant collagen molecule of claim 65, wherein the collagenous domain is sufficiently hydroxylated to form a triple-helical structure.

84. A bacterial cell according to claim 1 capable of producing a hydroxylated recombinant protein comprising a foldon domain of SEQ ID NO: 61, wherein the foldon domain is fused to a terminus of the hydroxylated recombinant protein and facilitates self-assembly of the protein into a triple-helical structure.

85. A bacterial cell according to claim 10 that produces a hydroxylated recombinant protein comprising a foldon domain of SEQ ID NO: 61, wherein the foldon domain is fused to a terminus of the hydroxylated recombinant protein and facilitates self-assembly of the protein into a triple-helical structure.

86. The method of claim 23, wherein the post-translationally hydroxylated recombinant protein comprises a foldon domain of SEQ ID NO: 61, wherein the foldon domain is fused to a terminus of the hydroxylated recombinant protein and facilitates self-assembly of the protein into a triple-helical structure.

87. The post-translationally hydroxylated recombinant collagen molecule of claim 36, comprising a foldon domain of SEQ ID NO: 61, wherein the foldon domain is fused to a terminus of the hydroxylated recombinant protein and facilitates self-assembly of the protein into a triple-helical structure.

88. The Gram-negative bacterial cell of claim 46, that is capable of producing a hydroxylated recombinant protein comprising a foldon domain of SEQ ID NO: 61, wherein the foldon domain is fused to a terminus of the hydroxylated recombinant protein and facilitates self-assembly of the protein into a triple-helical structure.

89. The method of claim 57 wherein the post-translationally hydroxylated recombinant protein comprises a foldon domain of SEQ ID NO: 61, wherein the foldon domain is fused to a terminus of the hydroxylated recombinant protein and facilitates self-assembly of the protein into a triple-helical structure.

90. The post-translationally hydroxylated recombinant collagen molecule of claim 65, comprising a foldon domain of SEQ ID NO: 61, wherein the foldon domain is fused to a terminus of the hydroxylated recombinant protein and facilitates self-assembly of the protein into a triple-helical structure.

91-93. (canceled)

94. An engineered bacterial cell-based system capable of expressing recombinant proteins comprising:
c) one or more nucleic acids encoding a sugar-1,4-lactone oxidase or a sugar-1,4-lactone dehydrogenase; and
d) one or more nucleic acids encoding an ascorbate-dependent biosynthetic enzyme,
wherein the nucleic acids are either genes inserted into the bacterial genome or plasmids.
